(19) United States
(12) Patent Application Publication
(10) Pub. No.: US 2024/0368552 A1
(43) Pub. Date: Nov. 7, 2024

(54) PRE-EPICARDIAL CELLS AND USES THEREOF

(71) Applicant: The General Hospital Corporation, Boston, MA (US)

(72) Inventors: Harald C. Ott, Wenham, MA (US); Jun Jie Tan, Boston, MA (US); Jacques Guyette, Worcester, MA (US)

(21) Appl. No.: 18/568,589

(22) PCT Filed: Jun. 9, 2022
(86) PCT No.: PCT/US2022/032832

§ 371 Date: Dec. 8, 2023

Related U.S. Application Data
(60) Provisional application No. 63/208,933, filed on Jun. 9, 2021.

(51) Int. Cl. C12N 5/077; A61K 35/34; A61P 9/10; C12N 5/00

(52) U.S. Cl. CPC C12N 5/0657; A61K 35/34; A61P 9/10; C12N 5/0037; C12N 2500/90; C12N 2501/00; C12N 2501/155; C12N 2501/165; C12N 2501/33; C12N 2501/385; C12N 2501/415; C12N 2506/45

(57) ABSTRACT

Methods for generating pre-epicardial cells (PECs) and/or cardiomyocytes (CMs) useful for cardiac tissue engineering, compositions comprising the cells, and methods of use thereof.

### CLAIM OF PRIORITY

This application claims the benefit of U.S. Provisional Patent Application Ser. No. 63/208,933, filed on Jun. 9, 2021. The entire contents of the foregoing are incorporated herein by reference.

### TECHNICAL FIELD

This disclosure relates to pre-epicardial cells (PECs), compositions comprising the cells, methods of generating PECs, and methods of use thereof.

### BACKGROUND

Cardiovascular disease (CVD) is the number one killer worldwide, with myocardial infarction (MI) responsible for approximately 1 in 6 deaths (1), and ischemic heart disease (IHD) as the leading single cause of death globally, responsible for over 15 million deaths in 2016. Further, congenital heart defects, which occur in nearly 14 of every 1000 newborn children, are the most common congenital defects and the leading cause of death in the first year of life. The heart is an organ that fails beyond repair, way too often, because of the intrinsic inability of the damaged heart tissue to regenerate after injury (2). Heart transplantation is hampered by donor shortage, life-long immunosuppression and its success rates are linked to the experience of the surgical team. Given the widespread nature of CVD and challenges in heart transplantation, there is a tremendous need for replacing damaged heart tissue and restoring cardiac function with functional cardiac grafts.

### SUMMARY

The present disclosure provides compositions and methods for producing pre-epicardial cells (PECs), and applications of PECs in cardiac tissue engineering, and the like. This disclosure is based, at least in part, on the findings that a premature form of epicardial cells (PECs) can be generated from human induced-pluripotent stem cells (hiPSCs) using methods disclosed herein. Further, hiPSC-derived PECs were surprisingly found to be functional and were able to interact with cardiomyocytes (CMs) to enhance the function and structural organization in in-vitro three-dimensional PEC/CM microtissues, thereby generating electrically active cardiac-microtissue constructs with distinct luminal structures.

In the first aspect, the disclosure features a method of generating a population of pre-epicardial cells (PECs), the method comprising: providing a population of induced pluripotent stem cells (iPSCs), preferably human iPSCs; culturing the population of cells at a density of about 5,000-500,000 cells per mm², preferably about 200,000 cells per mm²; performing the following steps in order: (a) treating the cells with a first medium comprising a Wnt signaling activator for about 1-4 days in the absence of insulin, preferably for about 48 hours; (b) replacing the first medium of step (a) with a second medium comprising insulin for about 24 hours; and (c) treating the cells with one or more of Bone Morphogenetic Protein 4 (BMP4), Retinoic Acid, and optionally vascular endothelial growth factor (VEGF), for about 3-7 days, preferably for about 4 days.

In some embodiments, the method further comprises the following steps in order between steps (b) and (c): (i) treating the cells with a Wnt signaling inhibitor for about 1-3 days, preferably about 48 hours in the second medium; and (ii) replacing the second medium with a third medium comprising insulin for about 1-3 days, preferably for about 48 hours.

In the second aspect, the disclosure features a method of generating a population of pre-epicardial cells (PECs) and cardiomyocytes (CMs), the method comprising: providing a population of induced pluripotent stem cells (iPSCs), preferably human iPSCs; culturing the population of cells at a density of about 5,000-500,000 cells per mm², preferably about 200,000 cells per mm²; performing the following steps in order: (a) treating the cells with a first medium comprising a Wnt signaling activator for about 1-4 days in the absence of insulin, preferably for about 48 hours; (b) replacing the first medium of step (a) with a second medium comprising insulin for about 24 hours; (c) treating the cells with a Wnt signaling inhibitor for about 1-3 days, preferably about 48 hours in the second medium; and (d) replacing the second medium with a third medium comprising insulin for about 1-3 days, preferably for about 48 hours; and (e) treating the cells with one or more signaling activators of Bone Morphogenetic Protein 4 (BMP4), Retinoic Acid, and optionally vascular endothelial growth factor (VEGF), for about 3-7 days, preferably about 4 days.

In some embodiments, the first and/or second medium is a serum-free medium. In some embodiments, the first and/or second medium is Roswell Park Memorial Institute (RPMI) 1640 medium. In some embodiments, the first medium RPMI medium with B-27 Supplement Minus Insulin.

In some embodiments, the Wnt signaling activator is provided in a range of about 8 to about 15 μM, preferably about 12 μM. In some embodiments, the BMP4 is provided in a range of about 25 to about 75 ng/ml, preferably about 50 ng/ml. In some embodiments, the VEGF is provided in a range of about 2 to about 7 ng/ml, preferably about 5 ng/ml. In some embodiments, the retinoic acid is provided in a range of about 2 to about 6 μM, preferably about 4 μM. In some embodiments, the Wnt signaling inhibitor is provided in a range of about 2 to about 7 μM, preferably about 5 μM. In some embodiments, the Wnt signaling activator is CHIR99021. In some embodiments, the Wnt signaling inhibitor is IWP-4.

In some embodiments, the PECs express one or more of the markers WT1, TBX18, SEMA3D and SCX within 7 days of generating PECs. In some embodiments, the PECs express one or more of the markers UPK1B, ITGA4, ALDH1A2 after 7 days of being generated, wherein the PECs are contact with CMs. In some embodiments, the PECs have one or more of the follow characteristics: (1) secrete IGF2; (2) stimulate CM proliferation; and (3) induce the formation of functional CM aggregates.

In some embodiments, the disclosure features a population of cells comprising preferably at least 60%, 70%, 80%, or 90% PECs made by the method of the disclosure. In some embodiments, the population of cells comprises PECs and CMs made by the method of the disclosure. In some embodiments, the population of cells comprises about 66% PECs and about 33% CMs. In some embodiments, the population of cells comprises about 50% PECs and about 50% CMs. In some embodiments, the population of cells comprises about 40% PECs, about 50% CMs and about 10% uncharacterized cells.

In some embodiments, the disclosure features a composition comprising the population of cells.

In some embodiments, the disclosure features a method of treating a subject who has or is at risk of developing a cardiovascular disease or has injured myocardial tissue, the method comprising: obtaining primary somatic cells, preferably from the subject who has or is at risk of developing cardiovascular disease, and generating iPSCs from the primary cells; generating a population of cells comprising PECs and optionally CMs by the method of the disclosure; and administering the population of cells to the subject.

In some embodiments, the cells are administered by being implanted directly into or near the affected area of the subject's heart. In some embodiments, the cells are administered directly via injection. In some embodiments, the cells are placed onto one or more degradable sheets implanted on the subject's heart. In some embodiments, the administration of the cells improves cardiac functionality.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D shows generation of cardiomyocytes (CMs) and PECs from BJ-RiPS cells.

FIG. 1A shows a schematic of the timeline of CHIR treatment to generate LPM and PECs from hiPSCs and FACS dotplots of PDGFRA and KDR expressions in LPM at day 3. Bar graph shows mean±SEM of analyzed from three independent experiments by flow cytometry.

FIG. 1B shows representative flow cytometric dot plots of WT1 expression in differentiating PECs at day 3 (LPM), day 5 and day 7. The bar graph represents the percent of WT1 at day 3 (LPM), day 5 (D5) and day 7 (D7) of differentiation. Significant difference between groups and p values were determined by one-way ANOVA with Tukey's multiple comparisons test. *indicates p<0.0001.

FIG. 1C is an RTPCR analysis of differentiated PEC at day 3, 5 and 7. Log₂ fold change in gene expression was normalized to the level in LPM. Data presented in mean±standard error of mean (SEM) from three independent experiments (n=3). One-way ANOVA with Dunnett post-hoc test was used in the statistical analysis. The presented adjusted p value in all figures corresponds to the group in the respective column versus LPM. ****indicates p<0.0001 versus LPM.

FIG. 1D shows representative immunofluorescence images of day 7 PECs from three independent differentiation demonstrating the expression of WT1, cTnT, CD31, SMA, ZO1 and TCF21. Scale bar=100 μm.

FIGS. 2A-2D show unsupervised hierarchical clustering and Principal component analysis of the cells.

FIG. 2A shows an unsupervised hierarchical clustering of 2000 most variable genes based on correlation distance (average linkage) method. Replicates for each group were from independent differentiation batches.

FIG. 2B shows PCA cluster PECs closer to EpiP1 as compared to EpiH9.

FIG. 2C shows MA plots of differential expression analysis for PEC vs EpiH9.

FIG. 2D shows MA plots of differential expression analysis for EpiP1 vs EpiH9 comparisons. Differentially expressed genes (DEGs) are indicated for upregulation (Up) and downregulation (Down) (adjusted p value<0.05 and absolute fold change ≥2). Functional clustering of gene ontology terms for biological processes for both up and downregulated DEGs for PEC vs EpiH9 and EpiP1 vs EpiH9 comparisons are shown. DEGs were determined using DeSeq2 and analyzed using the Wald test with Benjamini-Hochberg multiple comparison corrections. FDR value=0.05 was used as to determine the statistical significance.

FIGS. 3A-3E show the capability of PEC to undergo EMT.

FIG. 3A shows representative immunofluorescence characterization of PECs 6 days after treated with SB431542, TGFβ, bFGF, and TGFβ and bFGF from three independent experiments. WT1—bright white areas; ZO1—gray structures around bright white areas; DAPI—gray puncta; CD90—gray structures around gray puncta);

FIGS. 3B-3D show RTPCR analysis of epicardial genes (FIG. 3B), epithelial-mesenchymal transition genes (FIG. 3C), and mesenchymal/fibroblast genes (FIG. 3D) in PECs, after 6 days of stimulation. All expressions were normalized to SB-431542 (SB) treated PECs. Data presented in mean±SEM are from three independent experiments (n=3). Statistical significance between group was determined using one-away ANOVA with Dunnett post-hoc test. The presented adjusted p value in all figures corresponds to the group in the respective column versus SB-treated PECs. ####p<0.0001 versus SB-treated PECs.

FIG. 3E shows FACS analysis of CD90 expression in PECs 6 days after treated with SB431542, TGFβ, bFGF, and TGFβ+bFGF. Overlapping histograms presented in (i), representative FACS dot plots for each group and bar graph with mean±SEM presented in (ii) (n=3 independent replicates). Significant difference between group were analyzed using one-way ANOVA with Tukey's post hoc test. ****indicates p<0.0001.

FIGS. 4A-4H show PEC fate in CM coculture and its effect on CM.

FIG. 4A shows the log₂ fold change of PECs alone and fluorescence-sorted mCherry-tagged PECs from CM co-culture compared to the expression level in PECs at day 7, analyzed by RT-PCR. Data presented in mean±SEM from three independent experiments (n=3). Statistical significance versus day 7 PECs was determined using one-away ANOVA with Dunnett post-hoc test.

FIG. 4B shows representative images of immunofluorescence-staining from three independent experiments confirming the presence of WT1⁺ cells (top) along with α-sarcomeric actinin⁺ CM (α-SA) in coculture. ECAD⁺ epithelial cells and CNN⁺ smooth muscle cells were also formed in CM coculture (bottom). Scale bar=200 μm.

FIGS. 4C-4E show Venus-CM surface area coverage and aggregate formation in PEC-CM co-culture (FIG. 4C) compared to CM-only controls (FIG. 4D) over time in culture, with quantification of cell coverage and aggregate height (FIG. 4E). Scale, 500 μm. Cell coverage measurements were taken from broad-field areas (7.6 mm²) from three independent experiments. Two-tailed student t-test was used for both coverage (bar) and height (box plot) analyses; Data presented in box and whiskers plots represent the maxima, 75^{th} percentile, median, 25^{th} percentile and minima. Aggregate height measurements were taken from n=15 areas from three independent experiments; Data presented as mean±SEM. ****indicates p values<0.0001.

FIG. 4F shows representative calcium-signaling traces (using Fluo-3AM) recorded at Day 14 from a broad PEC-CM co-cultured area under (i) unpaced and (ii) paced conditions via point-stimulation (white circle with asterisk); Scale, 100 μm), demonstrating network connectivity at points 1-4, from three independent experiments.

FIG. 4G is a schematic showing the timeline for PEC/CM co-differentiation from LPM. Dotplot presents the proportion of WT1+ cells, cTnT+ cells, and double negative cells in co-differentiation culture (n=1), analyzed by flow cytometry.

FIG. 4H shows immunofluorescence images of co-differentiation culture show representations of spontaneous spatial organization of PECs and CMs in a ‘tube’ like structure from three independent experiments.

FIGS. 5A-5L show the effects of PECs on 2D CM co-culture.

FIG. 5A shows representative fluorescence and binary images from 5 independent replicates of vCMs alone and co-cultured with either HUVECs, human cardiac fibroblasts (HCFs), or PECs after 8 days. The graph shows vCM surface area coverage in co-culture with HUVECs, HCFs, and PECs compared to CM-only controls after 8 days. Scale, 500 μm. Cell coverage measurements at 8 days were taken from broad-field areas (7.6 mm², N=5 for each group); Data presented as mean±SEM and analyzed by using one-way ANOVA with Dunnett post-hoc test versus PEC/CMs only. ****indicates p value<0.0001.

FIG. 5B shows two-dimensional aggregate height analysis of all CM culture groups (CMs-alone, PEC-CMs, HUVEC-CMs, HCF-CMs) at day 8 of culture. N=15 aggregates examined over three independent experiments. Data are presented in box and whiskers plot, with lines indicate 25th, 50th and 75th percentiles and minima and maxima. One-way ANOVA with Dunnett post-hoc test versus PEC/CMs was used. ****indicates p value<0.0001.

FIG. 5C shows cardiomyocyte contractility metrics. Area strain of cardiomyocytes from different experimental groups, determined using high-speed HDM imaging analysis, with n=7 areas per CM group, 3 contractions analyzed per area examined over three independent replicates. Data are presented in box and whiskers plot, with box lines indicating 25th, 50th and 75th percentiles, and whisker lines indicating minima and maxima. One-way ANOVA with Dunnett post-hoc test versus PEC/CMs was used. ****indicates p value<0.0001.

FIG. 5D shows Young's modulus of cell-seeded gels from different experimental groups, determined by atomic force microscopy. Data were analyzed from n=41, 29, 48 and 47 areas of CMs only, CM+PECs, CM+HUVECs and CMs+HCF, respectively examined over three independent replicates.

FIG. 5E shows Day 14 contractility (force/area) of CMs cultured alone or in co-culture with PECs, HUVECs, or HCFs (n=7).

FIGS. 5F-5I show Day 14 Fluo-3AM calcium-signaling analysis of CMs cultured alone or in co-culture with PECs, HUVECs, or HCFs for amplitude (FIG. 5F), maximal velocity of calcium transient upstroke (FIG. 5G), and maximal velocity of calcium transient decay (FIG. 511). Representative calcium traces are depicted for comparison (FIG. 5I). (n=15 for each group, across all calcium transient metrics); FIG. 5D-5I Data are presented in box and whiskers plot, with box lines indicating 25th, 50th and 75th percentiles, and whisker lines indicating minima and maxima. Statistical significance at p<0.05 was determined using Kruskal-Wallis with Dunn's multiple comparison tests. ****indicates p value<0.0001.

FIG. 5J shows representative images of α-Sarcomeric actinin-stained in CM and PEC coculture (α-actinin stain with DAPI in arrows). Sarcomere length were measured manually from a total of 30 sarcomeres of 3 control CMs and 30 sarcomeres of 3 PEC cocultured CMs derived from 3 independent biological replicates. Line presented in the boxplot are 25th, 50th and 75th percentiles and minima and maxima. Comparison between CM alone vs PEC/CM were analyzed using two tailed T-test with Welch's correction.

FIG. 5K shows representative FACS analysis of mitochondria stained CMs in control (left) and PEC coculture (right). Bar graph showed percent of CMs with high mitochondria density (% CM-Mito^{high}). Two-tailed student t-test was used (n=3, p<0.002).

FIG. 5L shows CM size analysis comparing CM (n=129) vs PEC/CM (n=115). Line presented in the boxplot are 25th, 50th and 75th percentiles and standard deviation. Comparison between CM alone vs PEC/CM were analyzed using two tailed T-test with Welch's correction.

FIGS. 6A-6I show differentiation of ventricular-committing CM (VM) and IGF-RA Signaling in PEC induced VM proliferation.

FIG. 6A shows the timeline and protocol of ventricular-like CM differentiation from lateral plate mesoderm (LPM, Top), and its cardiac gene expression at day 30 (Bottom) from three independent differentiations. Data presented as mean±SEM. Unpaired t-test with Welch's correction was used for statistical comparison between groups.

FIG. 6B shows representative immunophenotypic characterization of ventricular-CM at day 30 from three independent differentiation. BMS=retinoid-x-receptor antagonist BMS189453.

FIG. 6C shows representative immunofluorescence images of Sarcomeric Actinin (aSA) EdU⁺ (bright dots; representatives shown as asterisks) ventricular CMs after coculturing with PECs from three independent experiments.

FIG. 6D shows representative flow cytometric quantification of cTnT⁺ Edu⁺ ventricular CM in PEC co-culture after 6 days. The percentage (24.5% or 41.1% as indicated) was calculated by taking the cell count in Q2 and dividing by 100% cTnT+ cells, or total events in Q1 and Q2 only (rectangular boxed region). Data presented in mean±SEM (n=3). Two-tailed student T test was used.

FIG. 6E shows the percentage of cTnT⁺ EdU⁺ VM in response to Linsitinib. Data presented in mean±SEM (n=3 independent experiments) versus 0 μM Linsitinib. One-way ANOVA with Dunnett post-hoc test was used.

FIG. 6F shows the dose-response examination of IGF2 on CM proliferation. Data presented in mean±SEM (n=3 independent experiments). One-way ANOVA with Dunnett post-hoc test was used.

FIG. 6G shows the ELISA analysis of IGF2 in conditioned medium 2 and 6 days after co-culturing with PECs. Data presented in mean±SEM (n=3 independent experiments). One-way ANOVA with Dunnett post-hoc test was used. ****indicates p value<0.0001 versus CM only.

FIG. 6H shows the expression of IGF2 RNA in PECs in response to increasing CM number. Data presented in mean±SEM (n=3 independent experiments). One-way ANOVA with Dunnett post-hoc test was used. ****indicates p value<0.0001 versus no CMs.

FIG. 6I shows IGF2 expression in PECs in CM co-culture in transwell with or without RARα/γ antagonist BMS-189453 (BMS). Data presented in mean±SEM (n=3 independent experiments). One-way ANOVA with Dunnett post-hoc test was used. ****p<0.0001 versus PECs-CM co-culture without BMS.

FIGS. 7A-7B show PEC organization on CM aggregates.

FIG. 7A shows z-stack images of the whole-mount PEC-CM aggregates were fluorescent stained for SMA, CD31, and DAPI of after 15 days (n=1). Scale=200 μm.

FIG. 7B shows immunostaining of day 15 CM only aggregates for MLC2V, SMA and DAPI. n=3 independent experiments, Scale bar=100 μm

FIGS. 8A-8I show stage-characterization of PEC differentiation.

FIG. 8A RTPCR Characterization of LPM at day 3 following 24 h and 48 h of CHIR99021 treatment, pluripotency markers (i.e. Oct3/4, SOX2 and NANOG), Data presented are mean fold change±SD (n=3).

FIG. 8B Primitive streak markers (i.e. MXL and T), cardiac progenitor markers (i.e. GATA4, ISL1, and Nkx2.5) and lateral plate mesoderm marker (i.e. Pitx2 and FoxF1) expressions at day 3, with fold change normalized to hiPSC expression at day 0. *p<0.001 versus hiPSCs. Data presented are mean fold change±SD (n=3).

FIG. 8C Representative immunofluorescent images of day 7 PECs stained for WT1 and ZO1. Scale=100 m.

FIG. 8D Representative FACS plot showing CD31 expression in PECs and HUVEC cells (n=1).

FIG. 8E RTPCR characterization of differentiating of PECS and CMs from LPM at day 5 and day 7. Data presented are mean fold change±SD normalized to LPM (n=3).

FIGS. 8F-8G FACS plot of day 7 PECs differentiated from hiPSC lines obtained from ATCC (F) and Gibco (G). Secondary stained population and WT1-stained population are indicated.

FIG. 8H Representative images of PEC in maintenance culture (N=3). Immunostaining of PECs maintained in SB-supplemented medium (left) and basal medium (right). Scale bar=100 μm. Smooth muscle actin (SMA; arrowheads); Calponin (CNN1; asterisks); DAPI (puncta).

FIG. 8I PEC maintenance with SB-supplemented medium and PEC medium (PECM). Schematic illustrates the timeline for sampling PECs after culturing with SB-supplemented medium and PECM for RT-PCR analysis. Immunostaining of TCF21⁺ and SMA⁺ as indicated. PECs after 6 days in PECM culture. Bar graphs show the expression of epicardial-related markers after culturing in PECM (at day 11) and SB supplemented medium (at day 13 and 18) relative to the expression in freshly differentiated PECs at day 7. *p<0.05, **p<0.001 versus PECs at day 7. Data presented are mean fold change±SD (n=3)

FIGS. 9A-9D shows RNA Sequencing analysis of PEC, Epi^{P1} and Epi^{DONOR}.

FIG. 9A K-means clustering of differentially expressed genes (DEGs) from Epi^{P1} vs Epi^{H9}, PEC vs Epi^{H9} and Epi^{DONOR} vs Epi^{SC} comparisons.

FIG. 9B Functional clustering of enriched gene ontology terms for biological processes for clusters A, B, D, F and G.

FIG. 9C Enrichment analysis using the exact hypergeometric probability test on the DEGs lists for PEC and Epi^{P1} with the proepicardial (proEP) geneset extracted from the Cui et al. (2019) study (3).

FIG. 9D Exact hypergeometric probability for overlapped differentially expressed genes (DEGs) and dbEMT2.0 curated genes. Individual list of DEGs from PEC vs Epi^{H9} (PEC, 2700 DEGs), Epi^{P1} vs Epi^{H9} (EpiP1, 903 DEGs) and Epi^{DONOR} vs Epi^{SC} (EpiDONOR, 1422 DEGs) analyses were used to compare with 1184 dbEMT2.0 curated genes to determine the statistical significance of the overlap between two groups of genes. All the unique genes found in both studies (28395 genes) were used as the background for the analysis.

FIGS. 10A-10G show PEC migration and its effect on CM in Coculture.

FIG. 10A The migration of mitomycin treated PECs (white dotted box) to the gap was imaged at 0 h, 12 h and 21 h with or without CM in co-culture. Data presented are mean±SD, n=3 each group.

FIG. 10B PEC recolonized area was measured after 12 h and 21 h with or without CM. Data presented are mean±SD, n=3 each group.

FIG. 10C Representative images of mCherry-PECs and Venus-CMs as indicated in co-culture at (i) day 0, day 1 and day 10. Immunofluorescence images of area-of-interest (black box) staining CM and PECs for WT1 and SMA after 10 days (ii-x).

FIG. 10D Representative images of PECs at CM border staining for WT1, smooth muscle actin and alpha sarcomeric actin (aSA) as indicated. Nuclei were stained with DAPI. Scale: 100 μm, n=3.

FIG. 10E Representative images of the re-isolated PECs from 6 days CM coculture showed WT1 and TBX18 expressions as indicated.

FIGS. 10F-10G Representative images of the PEC/CM co-differentiation culture showed WT1 and cTnT expressions as indicated. BF, bright field FIGS. 11A-11D show characterization of CM-Spheres.

FIG. 11A Sphere formation of undifferentiated human iPS cells in spinner flask culture at 24, 48, and 72 h at 10× magnification. Spheres at 48 h were used for cardiac differentiation. Scale, 200 μm.

FIG. 11B Schematic depicting the timeline for cardiac differentiation of iPS spheres.

FIG. 11C Gene expression of CM-spheres (day 15 after onset of differentiation) versus control iPS cells (i), and representative flow cytometry results indicating day 15 CM-spheres are >70% cTnT⁺. Secondary only-stained population is indicated in dashed oval.

FIG. 11D Histological analysis of day 15 CM-spheres, staining positive for a panel of cardiac-specific markers. Scale, 100 μm.

FIGS. 12A-12E show characterization of PEC spheres.

FIGS. 12A-12C CM spheres and PEC spheres in spin culture after 24-hr. PEC spheres, generated in spinner flask using PEC Medium with RPMI complete medium in a 1:1 ratio for 24 h, were collected and immuno-stained for SMA and WT1 as indicated after cytospin and fixation on microscopic slide. CM-spheres and PEC-spheres in static culture after 48 h.

FIG. 12D Calcium imaging analysis of a PEC-CM aggregate after 96 hours in co-culture, demonstrating repeated calcium wave propagations from area 1 (A1) to area 2 (A2). Scale, 100 μm.

FIG. 12E PEC Calcium transient. Day 7 PEC-only cultures were treated with Fura-2 dye, electrically paced (20 V, 0.6 ms, 0.7 Hz), and analyzed under the same methods used to test calcium transients of CM-only and PEC-CM co-culture groups. PECs did not demonstrate a calcium transient flux, suggesting that they did not actively contribute to the calcium transient differences observed between CM-only and PEC-CM cultures.

FIGS. 13A-13I show sphere-structure analysis for CM-spheres, and PEC-spheres, PEC-CM aggregates (n=3, each group)

FIG. 13A percent of spheres with structures over time during sphere formation.

FIG. 13B number of structures per sphere during sphere formation.

FIG. 13C average cross-sectional area of the luminal structures.

FIGS. 13D-13I Day 15 analysis: (FIG. 13D) a representative fluorescent image of a PEC-CM aggregate (scale, 500 μm), (FIG. 13E) sphere density, (FIG. 13F) average sphere area, (FIG. 13G) structure size/sphere size, (FIG. 13H) average structure cross-sectional area, and (FIG. 13I) number of structures per sphere.

FIGS. 14A-14D show PEC-CM co-culture in three-dimensional tissue format.

FIG. 14A Histology of cross-sections from Day 15 PEC-CM aggregates, CM-spheres, and PEC-spheres (n=3); including H&E representations above TUNEL staining analysis. Scales indicated.

FIGS. 14B-14C Quantification of sphere density and TUNEL positive cells in each culture generated from three independent replicates.

FIG. 14D vCM-spheres and mPEC-spheres integrate to form cardiac micro tissues with cellular complexity, demonstrating PEC-derived SMA⁺ and VE-Cadherin⁺ cells that form higher-order luminal structures, either lined with VE-cadherin⁺ cells (triangular arrowheads), or VE-cadherin⁺ cells layered with SMA⁺ cells (arrows). Figure and inset scales, both 100 μm. n=3, Scale bar=200 μm.

### DETAILED DESCRIPTION

Directed CM differentiation of hiPSCs employs systematic biochemical treatments to streamline definitive stages of cardiac development, generating spontaneously contracting cardiomyocytes (CMs) at a high efficiency in vitro (4-7). Human iPSC-derived CMs express sarcomeric proteins, exhibit ion channels, propagate cardiac-specific action potentials, and demonstrate excitation-contraction coupling capable of responding to electrical and biochemical stimuli (7, 8). However, hiPSC-derived CMs and resulting cardiac constructs remain phenotypically immature, with underdeveloped organization and electromechanical function (9, 10). Furthermore, bioengineered heart tissues using defined cardiac cells still lack the cellular and structural complexity of native myocardium.

The epicardium originates from the pro-epicardial organ, a transient organ that emerges from the lateral plate mesoderm (LPM), located proximal to the venous pole of the looping heart during development. The absence of the proepicardial organ or epicardium results in underdeveloped ventricles and embryo lethality, due to hindered CM proliferation, myocardial expansion, and coronary vessel formation (11-13). During heart maturation, epicardial-derived cells integrate within the myocardium and undergo epithelial-mesenchymal transition (EMT) to become fibroblasts, smooth muscle cells, and endothelial cells that enable healthy ventricular thickening, compaction, and angiogenesis (14-16). Epicardial-like cells have been generated from hiPSCs by modulating the bone morphogenic protein (BMP) and Wnt signaling pathway (17-19).

The methods of this disclosure demonstrate a simple method to generate pre-epicardial cells (PEC) from hiPSCs with high efficiency (86.8%), the premature form of epicardial cells expressing typical epicardial genes WT1, TBX18, SEMA3D and SCX but capable of developing further to a more mature epicardial cells (upregulated additional markers UPK1B, ITGA4, ALDH1A2) after being in contact with cardiomyocytes. PECs of the disclosure are highly migratory, can undergo epithelial-to-mesenchymal transition (EMT) with TGFβ or bFGF biochemical stimulation, but can also be phenotypically maintained in a proliferative state under TGFβ inhibition.

PECs of the disclosure have a unique RNA profile versus hiPSC-derived epicardial cells (20), in which RNA-Seq functional gene ontology analysis shows the PECs are earlier-stage and more plastic with respect to their key roles in cardiovascular development.

The findings recapitulate the developmental roles in embryonic heart formation, including epithelial-mesenchymal transition (EMT) and derivation of fibroblast and smooth muscle cells, and stimulation of ventricular myocyte proliferation partly via RA-dependent IGF signaling of CM proliferation. It was found that PEC differentiation is induced via BMP4, retinoic acid (RA), and vascular-endothelial growth factor (VEGF) signaling. This method also allows PEC/CM co-differentiation, and the latter reveals spontaneous PEC/CM spatial organization. During indirect co-culture using compartmentalized inserts, PECs showed enhanced migration in the presence of CM. Direct co-culture of PECs and CMs in monolayer generated CM networks with improved contractility and efficient calcium handling. Additionally, to the methods of the disclosure can be used to generate and observe interactions in three-dimensional constructs, spheroid co-culture, and allowed for the generation of electrically active cardiac-microtissue constructs with distinct luminal structures.

### Premature Epicardial Cells (PECs)

Pre-epicardial cells a.k.a. premature epicardial cells (PECs) of this disclosure, are cells derived from human induced pluripotent stem cells (hiPSCs) through two stages of differentiation: 1) Formation of lateral plate mesoderm, 2) Directed differentiation using three defined factors BMP4, RA, and VEGF. The pre-epicardial cells exhibit properties in stimulating cardiomyocyte proliferation, aggregation, and force contractility similar to the processes of in vivo myocardial expansion and growth driven by epicardium during early stage of heart development. These characteristics and capabilities are not seen using mature adult epicardial cells, hence may only be attributed to epicardial cells that are confined at early-stage development. The PECs are the premature form of epicardial cells prior to the exposure to signals deriving from the cardiomyocytes, or the niche which promote further cell development into forming more mature epicardial epithelium and the descendant epicardial-derived cells.

The PECs of the disclosure express one of more genetic markers, including but not limited to Wilm's Tumor (WT)1 (UniProtKB—P19544), T-Box Transcription Factor 18 (TBX18; UniProtKB—O95935), Semaphorin 3D (SEMA3D; UniProtKB—O95025) and Scleraxis BHLH Transcription Factor (SCX; UniProtKB—Q7RTU7) within 7 days of initiating the protocol to generate PECs from hiPSCs. In some embodiments, PECs are in contact with cardiomyocytes, and upregulate the expression of one or more genetic markers, including but not limited to, UPK1B (UniProtKB—O75841), ITGA4 (UniProtKB—P13612), ALDH1A2 (UniProtKB—O94788) after 7 days of being generated, wherein the PECs are contact with CMs.

Without being bound by theory, it is believed that the PECs of the disclosure have one or more of the following characteristics: PECs induce cardiomyocyte proliferation via RA-IGF2 signaling; PECs become more mature epicardial cells in cardiomyocyte coculture, or with specialized medium; Epithelial-mesenchymal transition is possible to derive fibroblast and smooth muscle cells in cardiomyocyte coculture; PECs induce cardiomyocyte aggregation and enhances cardiomyocyte electromechanical function in coculture; Co-differentiation of PECs and cardiomyocytes demonstrate spontaneous cardiomyocyte organization.

### Methods for Generating PECs

The PECs of this disclosure are generated from human induced pluripotent stem cells (hiPSCs). Methods to generate hiPSCs have been described in e.g., Mummery C L, et al. Circ Res. 2012 Jul. 20; 111(3):344-58 and are commercially available, e.g., STEMCELL Technologies. In one embodiment, PECs are generated by the following exemplary method, and analyzed as described in the Examples:

HiPSC culture, maintenance and differentiation. Human BJR-iPS cells (hiPSCs) were obtained from Harvard Stem Cell Institute, and maintained in MTeSR medium (STEMCELL Technologies). hiPSCs were passaged with ReleaSR (STEMCELL Technologies, Cambridge, MA) and plated at 1:20 ratio every 7 days onto 10 cm² dish pre-coated with growth factor reduced (GFR)-Matrigel (Corning, Tewksbury, MA). Briefly, hiPSCs were seeded at 200,000 cells/cm² onto a GFR-Matrigel-coated plate and maintained in MTeSR medium for 4-5 days. To generate the lateral plate mesoderm (LPM), the cells were treated with 12 μM Stemolecule™ CHIR99021 (Stemgent, San Diego, CA) in differentiation basal medium consisting of RPMI medium supplemented with B27 without insulin (Gibco, Grand Island, NY) (RPMI-INS,) for 48 h. Then, the medium was replaced with RPMI-INS for 24 h. Next, the culture was incubated with 5 μM of Stemolecule™ Wnt inhibitor IWP-4 (Stemgent, San Diego, CA) in RPMI-INS for 48 hr before switching it back to RPMI-INS, for additional 48 hr. After that, the medium was changed to RPMI medium supplemented with insulin-containing B27, and refreshed every 2 days for long term maintenance. For PEC differentiation, LPM cells at day 3 were treated with RPMI-INS supplemented 50 ng/ml bone morphogenic protein (BMP) 4 (PeproTech, Rocky Hill, NJ), 5 ng/ml vascular endothelial growth factor (VEGF; PeproTech, Rocky Hill, NJ) and 4 μM retinoic acid (RA; Stemgent, San Diego, CA) in RPMI-INS and changed every 2 days for 96 hr. PECs were dissociated using collagenase I (Sigma Aldrich, St. Louis, MO) and 1×TrypLE Express Enzyme (Gibco, Grand Island, NY), and maintained in complete PEC medium (PECM) consisted of DMEM/F12 supplemented with 1×insulin-selenium-transferrin (ITS; Gibco, Grand Island, NY), 5 ng/ml VEGF (PeproTech, Rocky Hill, NJ), 10 μM retinol, 4 μM RA (Stemgent, San Diego, CA) and 60 μg/ml ascorbic acid (Sigma). For plating, 1 μM of ROCK inhibitor Y-27632 dihydrochloride (Tocris Bioscience, Bristol, UK) was added for 24 h to facilitate cell attachment, after which medium was switched to PECM. For PEC maintenance, differentiated PEC were passaged and seeded at 10,000/well of 24 well-plate and maintained using SB medium consisted of DMEM/F12 supplemented with 0.4 mg/ml of Albumax (Gibco, Grand Island, NY), 1×ITS (Gibco, Grand Island, NY), 60 μg/ml ascorbic acid (Sigma Aldrich, St. Louis, MO), with 2 μM TGFβ inhibitor SB431542 (Tocris Bioscience, Bristol, UK). The proposed PEC differentiation was also repeated on two commercial lines, the Gibco hiPSC Episomal iPSC line reprogrammed from cord blood (Gibco, Grand Island, NY) and ATCC DYSO100 hiPSCs (ATCC ACS-1019) reprogrammed from foreskin fibroblast (ATCC, Manassas, VA). The same differentiation procedures and induction cocktails were used for both lines, but the CHIR99021 concentration was reduced to 6 μM for ATCC line during LPM induction. The specific compounds and reagents described here can be replaced by functional equivalents.

In some embodiments, the methods and compositions described herein utilize PECs and/or CMs that are differentiated in vitro from induced pluripotent stem cells. An advantage of using iPSCs to generate PECs and/or CMs for the compositions described herein is that the cells can be derived from the same subject to which the desired human cardiomyocytes and/or epicardial cells are to be administered. That is, a somatic cell can be obtained from a subject, reprogrammed to an induced pluripotent stem cell, and then re-differentiated into a human PEC and/or CM to be administered to the subject (e.g., autologous cells). Since the PECs and/or CMs (or their differentiated progeny) are essentially derived from an autologous source, the risk of engraftment rejection or allergic responses is reduced compared to the use of cells from another subject or group of subjects. In some embodiments, the PECs and/or CMs useful for the compositions described herein are derived from non-autologous sources. In addition, the use of iPSCs negates the need for cells obtained from an embryonic source. Thus, in one embodiment, the stem cells used to generate PECs and/or CMs for use in the compositions and methods described herein are not embryonic stem cells.

In some embodiments, the methods of this disclosure generate at least 60%, 70%, 80%, or 90% PECs. In some embodiments, the methods of this disclosure can generate PECs and CMs in the ratio of 1:1, 2:1, 3:1, 4:1, 5:1, 6:1, 7:1, 8:1, 9:1, 10:1, 1:2, 1:3, 1:4, 1:5; 1:6, 1:7, 1:8, 1:9 or 1:10. In some embodiments, the methods of this disclosure generate about 66% PECs and about 33% CMs. In some embodiments, the methods of this disclosure generate about 50% PECs and about 50% CMs. In some embodiments, the methods of this disclosure generate about 40% PECs, about 50% CMs and about 10% uncharacterized cells.

In some embodiments, the PECs and/or cardiomyocytes of the disclosure are generated from commercially available human embryonic stem cells. The embryonic stem cells can be obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines. Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the PECs and/or CMs described herein are not derived from embryonic stem cells or any other cells of embryonic origin.

In some embodiments, the disclosed methods are used to generate a population of pre-epicardial cells (PECs), the method comprising: providing a population of induced pluripotent stem cells (iPSCs), preferably human iPSCs; culturing the population of cells at a density of about 5,000-500,000 cells per mm², preferably about 200,000 cells per mm²; performing the following steps in order: (a) treating the cells with a first medium comprising a Wnt signaling activator for about 1-4 days in the absence of insulin, preferably for about 48 hours; (b) replacing the first medium of step (a) with a second medium comprising insulin for about 24 hours; and (c) treating the cells with one or more of Bone Morphogenetic Protein 4 (BMP4), Retinoic Acid, and optionally vascular endothelial growth factor (VEGF), for about 3-7 days, preferably for about 4 days.

In some embodiments, the disclosed methods are used to generate a population of a population of pre-epicardial cells (PECs) and cardiomyocytes (CMs), by providing a population of induced pluripotent stem cells (iPSCs), preferably human iPSCs; culturing the population of cells at a density of about 5,000-500,000 cells per mm², preferably about 200,000 cells per mm²; performing the following steps in order: (a) treating the cells with a first medium comprising a Wnt signaling activator for about 1-4 days in the absence of insulin, preferably for about 48 hours; (b) replacing the first medium of step (a) with a second medium comprising insulin for about 24 hours; (c) treating the cells with a Wnt signaling inhibitor for about 1-3 days, preferably about 48 hours in the second medium; and (d) replacing the second medium with a third medium comprising insulin for about 1-3 days, preferably for about 48 hours; and (e) treating the cells with one or more signaling activators of Bone Morphogenetic Protein 4 (BMP4), Retinoic Acid, and optionally vascular endothelial growth factor (VEGF), for about 3-7 days, preferably about 4 days.

In some embodiments, the medium used for culturing and/or treating the cells is a basal differentiation medium such as Roswell Park Memorial Institute (RPMI) 1640 (Gibco), a growth medium used in cell culture. Alternatively, other basal differentiation media known in the art, including commercially available differentiation media can be used. For example, in place of RPMI, DMEM, MEM, Ham's F-10 or F-12 formulations can be used.

A Wnt signaling activator is a molecule, e.g., antibody, protein, nucleic acid, or small molecule that activates the Wnt signaling pathway. Examples of Wnt signaling pathway activators include, but are not limited to, Lithium chloride, CHIR99021, SB-216763, and BIO (See, e.g., (21)), 2-amino-4-[3,4-(methylenedioxy)benzyl-amino]-6-(3-methoxyphenyl)pyrimidine, WAY-316606, (hetero) arylpyrimidines, IQ 1, QS 1 1, or dichloroacetate (DCA). Wnt agonists can also be obtained commercially from sources, such as Sigma-Aldrich, ApexBio, Santa Cruz Biotechnology, Cayman Chemicals, among others. In one embodiment, the Wnt agonist is CHIR99021. Wnt signaling activators focus on inhibiting GSK-3β, which normally disrupts the β-catenin destruction complex, allowing transportation of β-catenin into the nucleus to participate in gene transcription and expression. In some embodiments, the dose of a Wnt signaling activator used in the methods described herein, for example, is between about 8 to about 15 μM, for example, 12 μM.

Wnt inhibitors belong to small protein families, including sFRP, Dkk, WIF, Wise/SOST, Cerberus, IGFBP, Shisa, Waif1, APCDD1, and Tiki1. Their common feature is to antagonize Wnt signaling by preventing ligand-receptor interactions or Wnt receptor maturation. See e.g., Cruciat and Niehrs Cold Spring Harb Perspect Biol. 2013 March; 5(3): a015081. IWP-4 inhibits WNT signaling and secretion by inactivating Porcupine, a protein responsible for palmitoylating WNT proteins. Examples of Wnt inhibitors that can be used are provided in (22). Some non-limiting examples of Wnt antagonists include Wnt pathway inhibitor V (also known as (E)-4-(2,6-Difluorostyryl)-N,N-dimethylaniline), IWR-1 endo, IWP-2, CCT036477, XAV-939 (tankyrase inhibitor), and a peptide comprising the sequence t-Boc-NH-Met-Asp-Gly-Cys-Glu-Leu-C02H. In some embodiments, the dose of a Wnt signaling inhibitor used in the methods described herein, for example, is between about 2 μM to about 7 μM, preferably about 5 μM.

A Retinoic acid signaling pathway agonist/activator is a molecule e.g., antibody, protein, nucleic acid, or small molecule that activates RA signaling pathway. Examples of retinoic signaling pathway agonist/activator are all-trans retinoic acid (RA); 9-cis RA; TTNPB; Tazarotene; AC 261066; AC 55649; Adapalene; AM 580; AM 80; BMS 753; BMS 961; CD 1530; CD 2314; CD 437; Ch 55; Isotretinoin; and TTNPB (4-[(E)-2-(5,6,7,8-Tetrahydro-5,5,8,8-tetramethyl-2-naphthalenyl)-1-propenyl] benzoic acid). Retinoic signaling pathway agonist/activator binds or directly activate one or several of the Retinoic acid receptors (RARα, β and γ). Transduction relies on heterodimerization of RAR with RXR and direct binding of DNA at retinoic acid response elements (RAREs) sites. Retinoic signaling pathway activity can be monitored using biochemical reporter assays, including RARE-LacZ system which expresses beta-galactosidase under the control of the retinoic acid responsive element, or activation of target genes such as the one listed in (23-25). In some embodiments, the dose of a Retinoic acid signaling pathway agonist/activator used in the methods described herein, for example, is between about 2 to about 6 μM, preferably about 4 μM.

SB 4 is a potent BMP4 agonist that activates canonical BMP signaling and increases SMAD-1/5/9 phosphorylation. BMP signaling agonist sb4 activates BMP4 target genes (inhibitors of DNA binding, Id1 and Id3) canonical BMP signaling. In some embodiments, the dose of BMP4 or its agonist used in the methods described herein, for example, is between about 25 to about 75 ng/ml, preferably about 50 ng/ml.

Vascular endothelial growth factor (VEGF) is a heparin-binding, dimeric protein related to the PDGF/sis family of growth factors. In some embodiments, the dose of VEGF used in the methods described herein, for example, is between about 2 ng/ml to about 7 ng/ml, preferably about 5 ng/ml.

The intracellular pathways activity of Wnt, retinoic acid, BMP 4 and VEGF can be monitored using biochemical reporter assays and for transduction cascade activation, known in the art.

Applications of PECs and/or CMs and Methods of Treatment

The methods and compositions described herein have various applications in cardiac tissue engineering including heart regeneration and ex vivo heart morphogenesis, cardiac cellular therapy, myocardial tissue engineering, disease modeling, and drug screening for treating the failing heart. For instance, the methods and compositions of the disclosure can be used to treat cardiovascular diseases, including but not limited to, myocardial infarction, ischemic heart disease, heart muscle disease, heart valve disease, pericardial disease, stroke, cardiomyopathy, congenital heart defect (e.g., non-compaction cardiomyopathy), hypertrophic cardiomyopathy, dilated cardiomyopathy, myocarditis, heart failure, and cardiomegaly and rheumatic heart disease. The PECs and/or PEC/CM co-culture or co-differentiation systems of the disclosure can be used to engineer a functionally and structurally more mature and complex heart tissue.

The heart is made of three major tissue layers: the endocardium, myocardium, and epicardium. The epicardium is the outermost epithelial layer of the heart and is responsible for the formation of coronary vascular smooth muscle cells. The epicardium can be re-activated to a more fetal form and/or the epicardial cells can undergo epithelial-to-mesenchymal transition (EMT) in response to an acute injury to the myocardium (e.g., a myocardial infarction). Provided herein are pre-epicardial cells and uses thereof (e.g., co-administration with cardiomyocytes) in the treatment of cardiac injury, cardiac disease/disorder, and/or promoting vascularization and engraftment of coadministered cardiomyocytes.

The cells can be administered using methods known in the art. In some embodiments, the cells are administered by being implanted directly into or near the affected area of the subject's heart. In some instances, the cells can be administered directly via injection. In some instances, the cells are placed onto one or more degradable sheets implanted on the subject's heart. The administration of the cells can improve cardiac functionality.

As used herein, the terms “administering,” “introducing” and “transplanting” are used interchangeably in the context of the placement of cells, e.g. PECs and/or CMs, as described herein into a subject, by a method or route which results in at least partial localization of the introduced cells at a desired site, such as a site of injury or repair, such that a desired effect(s) is produced. The cells e.g. PECs and/or CMs can be implanted directly to the heart, or alternatively be administered by any appropriate route which results in delivery to a desired location in the subject where at least a portion of the implanted cells or components of the cells remain viable. The period of viability of the PECs and/or CMs after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes do not proliferate to an extent that the heart can heal from an acute injury comprising cardiomyocyte death. In other embodiments, the cells can be administered via an indirect systemic route of administration, such as an intraperitoneal or intravenous route.

When provided prophylactically, the PECs and/or CMs can be administered to a subject in advance of any symptom of a cardiac disorder, e.g., heart failure due to prior myocardial infarction or left ventricular insufficiency, congestive heart failure etc. Accordingly, the prophylactic administration of a population of PECs and/or CMs serves to prevent a cardiac heart failure disorder or maladaptive cardiac remodeling, as disclosed herein.

Exemplary modes of administration for use in the methods described herein include, but are not limited to, injection, intracardiac delivery, systemic administration and implantation (with or without a scaffold material). “Injection” includes, without limitation, intracardiac, intravenous, intramuscular, intraarterial, intradermal, intraperitoneal and subcutaneous.

In some embodiments, a therapeutically effective amount of PECs and/or CMs is administered using direct injection into the heart including, but not limited to administration during open-heart surgery or by intracardiac injection through an intact chest. In some aspects of these methods, a therapeutically effective amount of PECs and/or CMs re administered using a systemic, such as an intraperitoneal or intravenous route. In other aspects of these methods, a therapeutically effective amount of PECs and/or CMs is administered using systemic or intraperitoneal administration. These methods are particularly aimed at therapeutic and prophylactic treatments of human subjects having, or at risk of having, a cardiac disease or disorder. The human PECs and/or CMs described herein can be administered to a subject having any cardiac disease or disorder by any appropriate route which results in an effective treatment in the subject. In some embodiments of the aspects described herein, a subject having a cardiac disorder is first selected prior to administration of the cells.

In some embodiments, an effective amount of PECs and/or CMs are administered to a subject by intracardiac administration or delivery. As defined herein, “intracardiac” administration or delivery refers to all routes of administration whereby a population of cardiomyocytes and/or epicardial cells is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

In some embodiments, an effective amount of PECs and/or CMs is administered to a subject by systemic administration, such as intravenous administration.

As used herein, the term “treating” includes reducing or alleviating at least one adverse effect or symptom of a condition, disease or disorder. For example, the term “treating” and “treatment” refers to administering to a subject an effective amount of a composition, e.g., an effective amount of a composition comprising a population of PECs and/or CMs so that the subject has a reduction in at least one symptom of the disease or an improvement in the disease, for example, beneficial or desired clinical results (e.g., improved cardiac function in an infarcted area of the heart, improved engraftment of cardiomyocytes etc.). For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. In some embodiments, treatment can include prophylaxis. However, in alternative embodiments, treatment does not include prophylaxis.

“Treatment” of a cardiac disorder, a cardiac disease, or a cardiac injury (e.g., myocardial infarction) as referred to herein refers to therapeutic intervention that enhances cardiac function and/or enhances PEC and/or CM engraftment and/or enhances PEC and/or CM transplant or graft vascularization in a treated area, thus improving the function of e.g., the heart. That is, cardiac “treatment” is oriented to the function of the heart (e.g., enhanced function within an infarcted area), and/or other site treated with the compositions described herein. A therapeutic approach that improves the function of the heart, for example as assessed by measuring left-ventricular end-systolic dimension (LVESD)) by at least 10%, and preferably by at least 20%, 30%, 40%, 50%, 75%, 90%, 100% or more, e.g., 2-fold, 5-fold, 10-fold or more, up to and including full function, relative to such function prior to such therapy is considered effective treatment. Effective treatment need not cure or directly impact the underlying cause of the heart disease or disorder to be considered effective treatment.

Indicators of cardiac disease or cardiac disorder, or cardiac injury include functional indicators or parameters, e.g., stroke volume, heart rate, left ventricular ejection fraction, heart rate, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

### Screening Assays:

Compositions comprising PECs and/or CMs as described herein can be used in screening assays for determining the toxicity, or alternatively the efficacy of a bioactive agent on cardiomyocyte viability, cardiomyocyte maturation, cardiomyocyte electroconductivity etc. The use of e.g., a co-culture of PECs and/or CMs more closely mimics the tissue of an intact heart than simply culturing cardiomyocytes alone. In particular, adult cardiomyocytes are difficult to culture as they do not reproduce and thus cannot be expanded in vitro. Thus, differentiation of human induced pluripotent stem cells to PECs in vitro and their subsequent maturation using a co-culture of PECs and/or CMs is especially useful in producing PECs and/or CMs for screening bioactive agents for the treatment of disease, or to monitor cell toxicity of a variety of agents.

In some embodiments, a co-culture of PECs and/or CMs comprises a 3-dimensional cell culture, or are cast in a tissue construct.

In some embodiments, co-cultured human PECs and/or CMs can be used in methods, assays, systems and kits to develop specific in vitro assays. Such assays for drug screening and toxicology studies have an advantage over existing assays because they are of human origin, do not require immortalization of cell lines, nor do they require tissue from cadavers, which poorly reflect the physiology of normal human cells. For example, the methods, assays, systems, and kits described herein can be used to identify and/or test agents that can promote cardiomyocyte maturation (e.g., as assessed by measuring sarcomere length), cell viability, cardiomyocyte electroconductivity (e.g., morphologically beating in unison or near-unison; expression of connexin 43; propagation of an action potential when stimulated with an electrode) etc. In addition, or in the alternative, the methods, assays, systems, and kits can be used to identify and/or test for agents useful in treating a cardiac disease or disorder, or for preventing/treating a cardiac injury (e.g., cardiac hypertrophy, heart failure etc.).

Accordingly, provided herein are methods for screening a test compound for biological activity, the method comprising (a) contacting a co-culture of human PECs and/or CMs with a test compound and (b) determining any effect of the compound on the cell(s) or a desired cell parameter. The effect on the cell can be one that is observable directly, or indirectly by use of reporter molecules.

### EXAMPLES

The invention is further described in the following examples, which do not limit the scope of the invention described in the claims.

### Materials and Methods

The examples used the following materials and methods.

Stimulation of epithelial-mesenchymal transition in PECs. Day 7 PECs were seeded at 100,000 cells/well on 24-well-plate pre-coated with 0.1% gelatin, and allowed to attach overnight in basal medium (BM) consisted of DMEM/F12 supplemented with 0.4 mg of Albumax (Gibco, Grand Island, NY), 1×ITS (Gibco, Grand Island, NY), 60 μg/ml ascorbic acid (Sigma Aldrich, St. Louis, MO). 1 μM of ROCK inhibitor Y27632 (Tocris Bioscience, Bristol, UK) was added to facilitate PEC attachment for 24 h. Then, PECs were expanded to full confluency in the BM with 2 μM SB431542 (Stemgent, San Diego, CA) but without AlbuMAX II (Gibco, Grand Island, NY), for 3-4 days. EMT were initiated by adding 5 ng/ml TGFβ (R&D system, Minneapolis, MN) for the initial 3 days and 10 ng/ml bFGF (PeproTech, Rocky Hill, NJ) for the subsequent 3 days or just bFGF alone, up to 6 days. Controls were epicardial cells in basal medium or with addition of 2 μM SB431542 (Stemgent, San Diego, CA).

Generation of mCherry or Venus labeled iPS cells. LIPOFECTAMINE 2000 transfection reagent (Invitrogen, Carlsbad, CA) was used for the transfection of 293T cells with lentivirus vectors carrying either mCherry or Venus gene and packaging vectors. Medium of transfected 293T cells was collected 72 hrs after transfection, then filtered with 0.45 um filter and concentrated. BJRiPS cells were trypsinized into single cells then infected with virus medium for 8 hrs. Infected cells were expanded for 2 rounds of FACS sorting to get a pure labeled population over 99%.

Quantitative PCR. Cells were lysed with RT buffer and total RNA was purified using RNeasy® Plus Mini kit (Qiagen, Hilden, Germany) which was reverse transcribed to cDNA using Superscript IV VILO Master Mix (Gibco, Grand Island, NY) and ran on Bio-rad T100™ Thermal Cycler according to manufacturer's protocol. Quantitative PCR was performed on StepOnePlus Real-time PCR system (Applied Biosystems, Foster City, CA) after mixing cDNA with Taqman Gene Expression Master Mix (Cat #: 4369016) and gene Taqman probe (Applied Biosystems, Foster City, CA) which can be found in Table 1. All gene expressions were normalized to housekeeping gene beta-actin and presented as log 2fold change (delta-delta CT).

Flow cytometry. Cells were dissociated using trypsin, pelleted by centrifugation at 300 g for 5 min, and fixed with Fixation/Permeabilization Solution Kit according to manufacturer's protocol (BD Biosciences, San Diego, CA). Cells were washed with 2 times of 10% BD Perm/Wash Buffer (BD Biosciences, San Diego, CA) to remove fixative prior to staining. Samples were incubated with primary antibody diluted in BD Perm/Wash Buffer at 4° C. for 45 min. To remove excessive primary antibody, samples were spun at 250 g for 5 min and washed once with Perm/Wash buffer prior to labelling with Alexa Fluor 488 secondary antibody (Molecular Probes Eugene, OR) at 1:500 for 30 min. All samples were washed once with BD Perm/Wash Buffer prior to analysis with BD Accuri, BD FACS LSR Flow Cytometer (BD Biosciences, San Diego, CA) or NovoCyte Flow Cytometer (ACEA Biosciences, San Diego, CA). All flow cytometric analyses were performed using FlowJo 10.5.0 software.

Immunofluorescence labelling and microscopy (cells, spheres, aggregates). Cultured cells were fixed with cold methanol at 4° C. for 15 min, and then washed with PBS. CM-spheres, PEC-Spheres, and cardiac aggregates were fixed overnight with 4% paraformaldehyde, embedded in Histogel™ (Thermo Scientific, Kalamazoo, MI), dehydrated, and embedded in paraffin. Sections were cut at 5 μm, deparaffinized, and treated with a heat-mediated antigen retrieval technique that included a 20-min boilin 0.01 M citrate buffer (pH 7.0). Both cells and histological sections were labelled with primary antibodies (Table 1) overnight at 4° C. Donkey anti rabbit AlexaFluor 488 and donkey anti-mouse AlexaFluor 546 and 647 (Molecular Probes Eugene, OR) were used as secondary antibodies at 1:500 and nuclei were counterstained with DAPI (Sigma Aldrich, St. Louis, MO). All labelled cells were visualized using Nikon Eclipse Ti fluorescence microscope (Nikon Instruments, Melville, NY), and processed with NIS element imaging software or ImageJ.

CM sarcomere length measurement. Control CM and PEC/CM were fixed and stained with sarcomeric α-activin after 6 days in 2D coculture. Sarcomere length was measured manually using ImageJ based on images taken at 20× magnification using Nikon Eclipse Ti fluorescence microscope (Nikon Instruments, Melville, NY).

PEC-CM migration and co-culture. In order to distinguish between CMs and PECs in co-culture, all CMs were differentiated from Venus-tagged BJR-iPS cells and maintained in RPMI supplemented with insulin containing B27. CMs from day 15 to 30 cultures were dissociated with collagenase, detached from culture plate with TypLE express with 63 U of DNase I ((Invitrogen, Carlsbad, CA) were purified from non-myocyte cells using Percoll gradient separation method as published prior to use 65. Day 7 PECs were produced using mCherry-tagged BJR-iPS cells. For migration assay, 50,000 of purified CM or 100,000 differentiated PECs were seeded into 2-well cell migration inserts (Ibidi USA, Fitchburg, WI) attached on 24-well plates. Inserts were removed after 24 h to allow complete cell attachment, and images were taken at baseline, 17 h and after 30 h. For CM-PEC co-culture, CMs were generated from Venus-tagged BJR iPS cells and were seeded at 250,000 cells per well in 24 well plate 2 days prior to seeding with mCherry-tagged PECs at 250,000 cells per well. The culture was maintained in insulin-supplemented B27 in RPMI and PECM at 1:1 ratio. Changes in CM morphology were monitored and captured after 8, 16 and 21 days.

PECs and CM were seeded onto the migration inserts (Ibidi USA, Fitchburg, WI) and allowed to attach for 24 h. Mitomycin C (10 μg/ml) please check was added for 2 hr prior to lifting the migration inserts. Cell movement toward the generated gap were imaged and captured at designated time using Nikon Eclipse Ti Fluorescence Microscope. Area recolonized by PECs were analyzed using ImageJ.

RNA Sequencing. Samples were preserved in Trizol Reagent (Invitrogen Life Technologies, Carlsbad, CA) and sent for RNA extraction and analysis by Girihlet Inc. RNA Integrity Number (RIN) was determined by RNA Nano Bioanalyzer. RNA library was generated using TruSeq RNA Library Prep Kit with 80 bp single end illumina sequencing (illumina, San Diego, CA). All fastq files and processed data were uploaded to Gene Expression Omnibus (GEO) database (GSE148543) (26, 27)

The sequencing data were uploaded to the Galaxy web platform and were pre-processed at the public server at usegalaxy.org.68. Fastq files were read and trimmed by using Trimmomatic (28) First 10 bases were clipped and sequences below the average Phred score of 30 within a sliding window of 4 bases were trimmed. Results from individual samples were aggregated using MultiQC (29). Based on the analysis, samples EpiP1_1 (GSM4473367) consisted of sequences with exceptionally high GC content (70%) and skewness and short length (43-44 bp) when compared to the rest of the libraries (50-54% GC and 51-60 bp in length), thus were removed from further analysis. Sequences from passed samples were mapped to hg38 human reference genome using Burrows-Wheeler Alignment tool for short sequences (<100 bp) (30). Generated BAM files were merged accordingly (31) and subsequently used to produce gene counts using featureCounts v 1.6.4 based on simple Illumina analysis mode (32).

We used iDEP.90, an integrated web application for differential gene expression and functional ontology analyses (33). Only genes with a minimum of 5 counts per million (CPM) in all libraries were considered (34). A total of 2000 most variable genes were included for hierarchical clustering based on the correlation (average linkage) method. A cut-off Z score of 4 was used. DESeq2 (idep ver 0.92) was used to determine the differential gene expression between 2 groups with FDR cut-off of 0.05 and minimum fold-change of 2 (35). Enrichment analysis of differentially expressed genes (DEGs) based on GO terms for biological processes was also performed (36).

To compare the transcriptomes of PECs and EpiP1 to previously published human primary-derived epicardial cells, we downloaded the fastq files from GSE84085 deposited in Gene Expression Omnibus (GEO) (20). To avoid batch effects between the studies, the sequences were pre-processed and analysed according to the same pipeline described above. The list of DEGs from this study (PEC vs EpiH9 and EpiP1 vs EpiH9) and Bao et al., (2016) (32) (EpiDONOR vs EpiSC) were used for both hierarchical clustering, PCA and enrichment analysis based in GO terms for biological processes.

Differentiation of Ventricular-like CM population. Human iPS cells were maintained and differentiated to LPM with Stemolecule™ CHIR99021 for 48 h. BMS-189453 (1-2 μM, Sigma Aldrich, St. Louis, MO) was supplemented together with Stemolecule™ Wnt inhibitor IWP-4 (Stemgent, San Diego, CA) in RPMI-INS for 48 h starting day 3. Then, BMS-189453 treatment was continued after additional 48 h after withdrawal of Stemolecule™ Wnt inhibitor IWP-4 in RPMI-INS until day 9. Culture medium was then switched to RPMI supplemented with insulin containing B27 to further maintain the differentiated ventricular myocytes until use.

Ventricular-CMs proliferation in PEC, HCF and HUVEC co-culture. Generated ventricular-CMs at day 25-30 were used in this experiment. Differentiated ventricular-CMs were digested with collagenase and TrypLE express solution containing 63 U/ml DNase1, enriched by Percoll® PLUS (GE-Amersham Biosciences, Uppsala, Sweden) density gradient separation method 1 and reseeded at 2.5×105 cells/well onto 24 well-plate in RPMI-INS+10% FBS. Day 7 PECs were seeded at 2.5×105 cells/well after 2-4 h of VM seeding. Then, media were refreshed with 10% Matrigel supplemented with RPMI-INS:PECM without RA at 1:1 ratio. Medium was refreshed every two days up to 6 days. Proliferative ventricular-CMs were labeled fixed and permeabilized with Fixation/Permeabilization Solution Kit (BD San Diego, CA), labelled for mouse anti human cTnT (1C11) (Abcam, Cambridge, MA) and counter-labelled with Click-iT™ Plus EdU Alexa Fluor™ 647 Flow Cytometry Assay Kit (Invitrogen, Carlsbad, CA) according to manufacturer's protocol. EdU and cTnT double positive cells were quantified with FACS LSRII flow cytometer (BD San Diego, CA). All analysis was performed using FlowJo software. IGF2 ELISA. Conditioned media from 6 days PEC-Ventricular CM co-culture was collected at day 6 and frozen at −40° C. prior to analysis. IGF2 in conditioned medium was analyzed using Human IGF-II Quantikine ELISA kit (R&D systems, Minneapolis, MN) in accordance with manufacturer's protocol.

IGF signaling in PEC-induced ventricular-CM Proliferation. Ventricular-CMs were seeded at 2.5×105 cells/well onto 24 well-plate in RPMI medium supplemented with B27 minus insulin+10% FBS. VMs were allowed to attach and the media were refreshed after 24 h. IGF1R Inhibition assay: every day with RPMI-INS containing 0, 0.06, 0.125, 0.25, 0.5 and 1 nM Linsitinib for 3 days. Proliferative VMs were labelled with cTnT antibody and Click-iT™ Plus EdU Alexa Fluor™ 647 Flow Cytometry Assay Kit (Invitrogen, Carlsbad, CA) prior to quantification using FACS LSRII flow cytometer (BD Bioscience, San Diego, CA) as mentioned previously.

IGF2 expression in PECs in response to ventricular-CMs: mCherry-PECs were seeded at 2.5×105 cells/well with increasing Venus-CM number (0, 63000, 125000, 250000, 250000, 500000) in 24 well plate, with medium change every day, up to 6 days. mCherry-PECs were separated from Venus-CM using BD FACS Aria Cell Sorter (BD Bioscience, San Diego, CA). Sorted mCherry-PECs were collected for RNA isolation and analysis, as described in earlier section (Quantitative PCR).

IGF2 ELISA. Ventricular-CMs were seeded at 2.5×105 cells/well onto 24 well-plate in RPMI medium supplemented with B27 minus insulin+10% FBS. VMs were allowed to attach and the media were refreshed after 24 h. mCherry-PECs were seeded at 2.5×105 cells/well. Conditioned media were harvested from PECs, CM and PEC-CM coculture after 48 h of incubation at 37° C. and kept in −80° C. until analysis. IGF2 protein were quantified using Human IGF-II/IGF2 Quantikine ELISA kit DG200 (R&D Systems, Minneapolis, MN). Samples were diluted 2-4 folds and IGF-II were quantified based on a standard log/log curve-fit with mean absorbance reading on the y-axis against the concentration on the x-axis. The optical density of each samples was obtained using Synergy HTX multi-mode reader (BioTek, Winnooski, VT).

Inhibition of RA signaling in PEC in Transwell: PECs were seeded at 2.5×105 cells/well in 24 well-plate while 2.5×105 cells VM were cultured in the transwell insert. Both cells were cultured using PECM:RPMI-INS medium, with BMS-189493 (5 μM) only present in lower compartment with PECs, for 6 days. IGF2 RNA was harvested from PECs for qPCR analysis.

Mitochondria staining: Mitochondria from isolated CMs, cultured either alone or in co-cultured with PECs, were stained using Mitochondrial Staining Kit (Abcam, ab176747). Dissociated CMs were resuspended in pre-warmed RPMI+B27 with 10% FBS stained with the dye working solution from the kit and incubate for 37° C. for 30 min for 2 h. The stained CMs were washed DPBS twice and then analyzed using flow cytometer (BD LSRII).

Measurement of sarcomere length: Dissociated and sorted CMs were seeded onto Fibronectin-coated 8 well chamber slide, cultured for 5 days and then fixed with 4% PFA. Cells were stained with α-actinin (Creative Diagnostics, DCABH-9438) and Goat anti-Rabbit IgG (H+L) Highly Cross-Adsorbed Secondary Antibody, Alexa Fluor 488 (Invitrogen), and imaged were acquired using the A1R confocal microscopy (Nikon). Sarcomere length was measured by the distance between the intensity peaks using FIJI/ImageJ software (ImageJ 1.53c, NIH). For CMs from PEC/CM co-culture, 373 sarcomeres from 16 CMs were analyzed. For CMs from monoculture, 230 sarcomeres from 30 CMs were analyzed (three independent replicates).

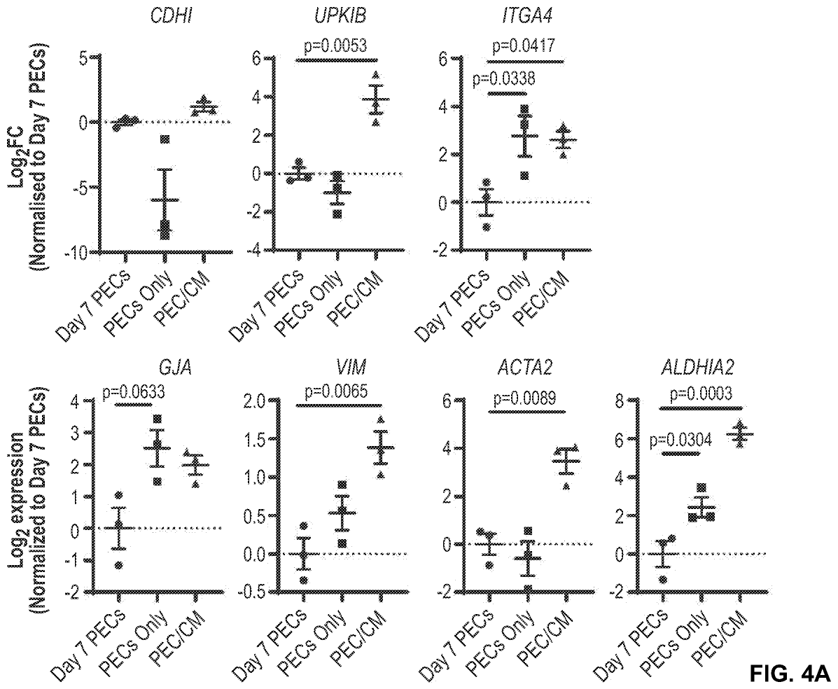

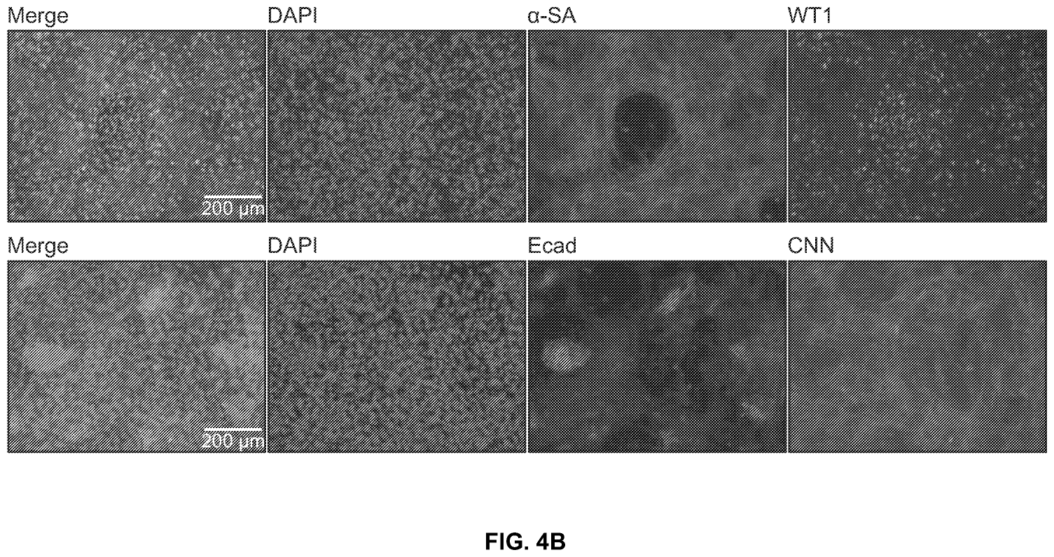

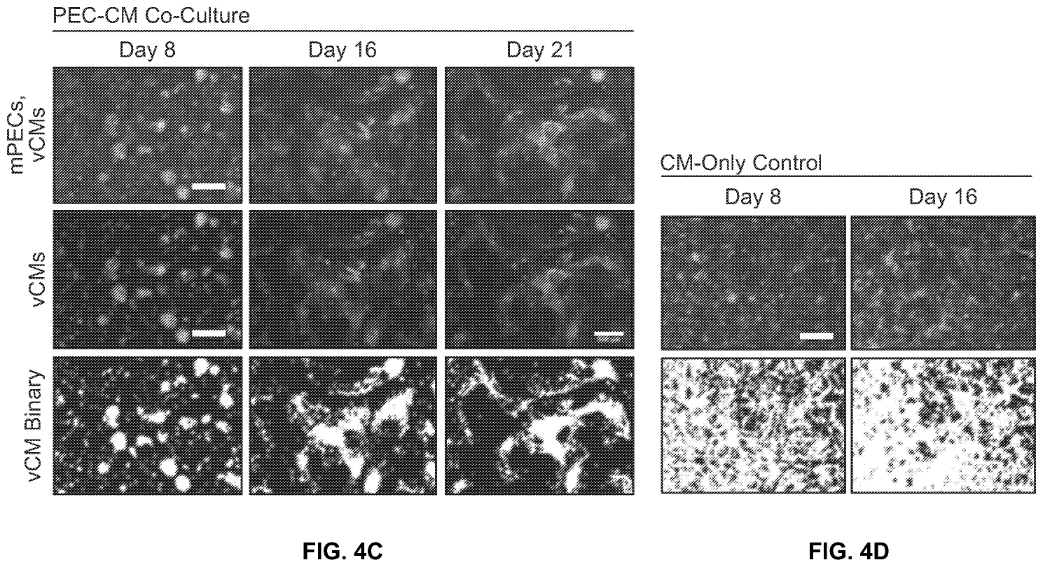

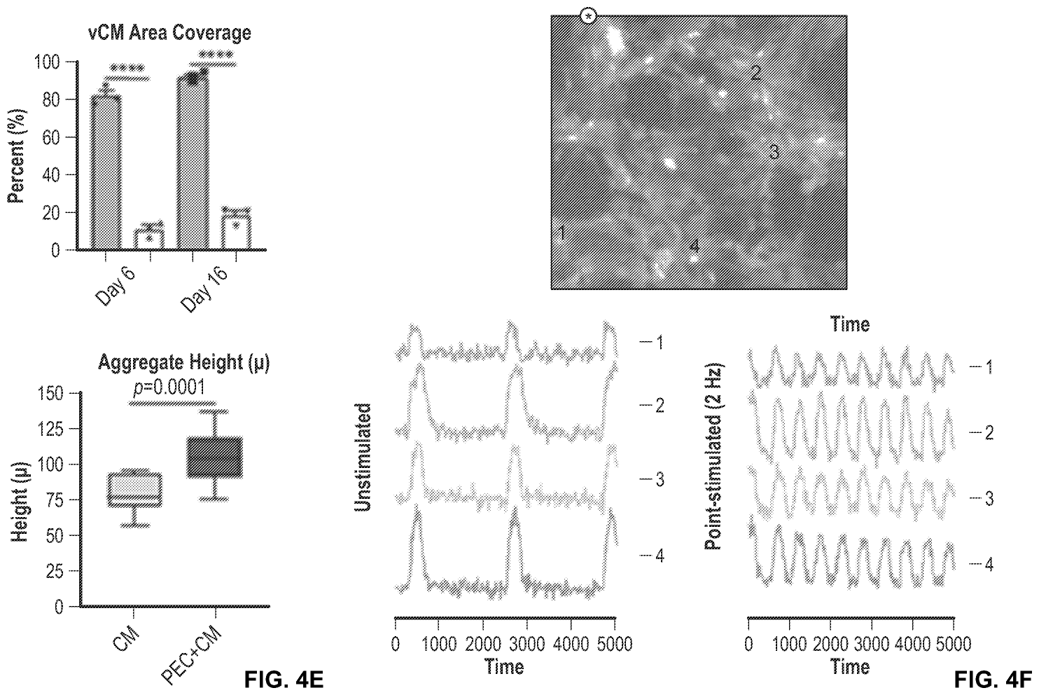

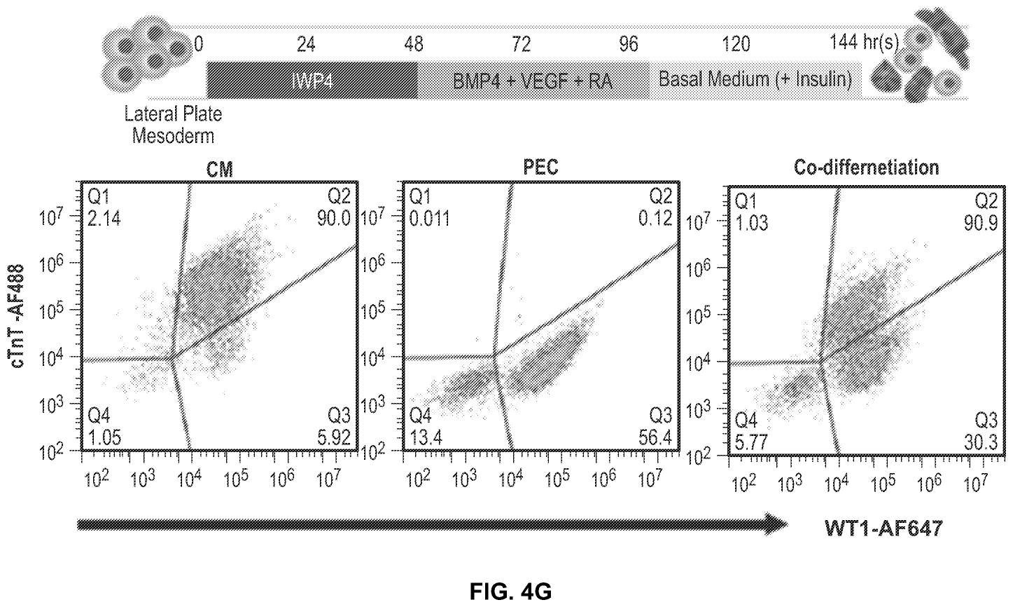

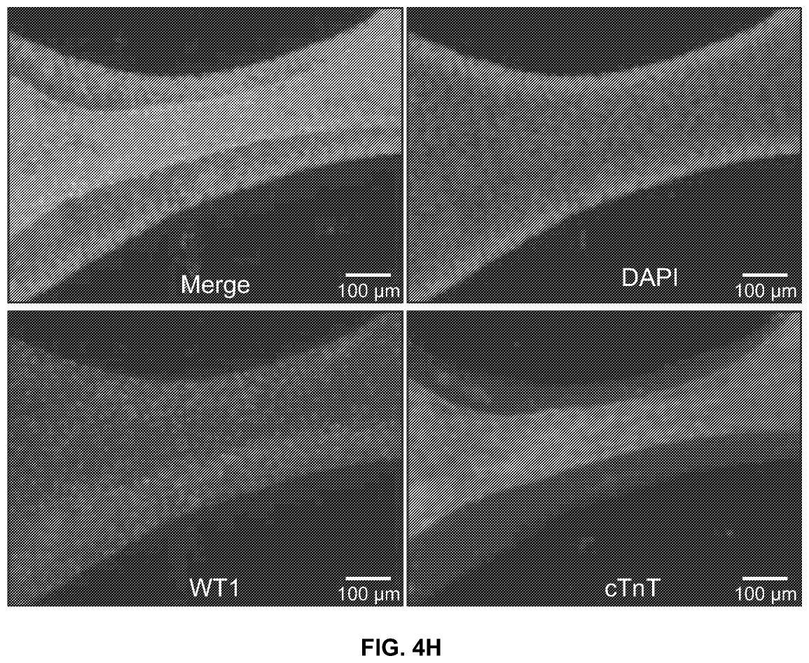

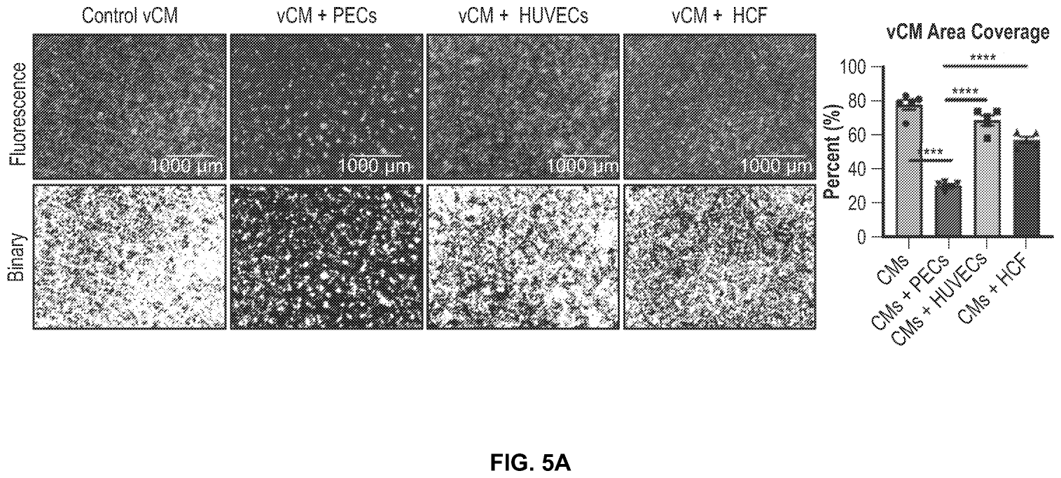

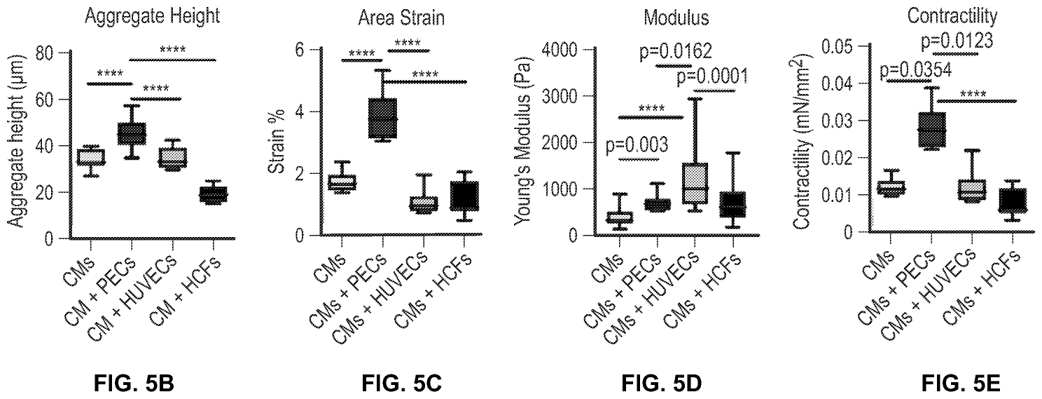

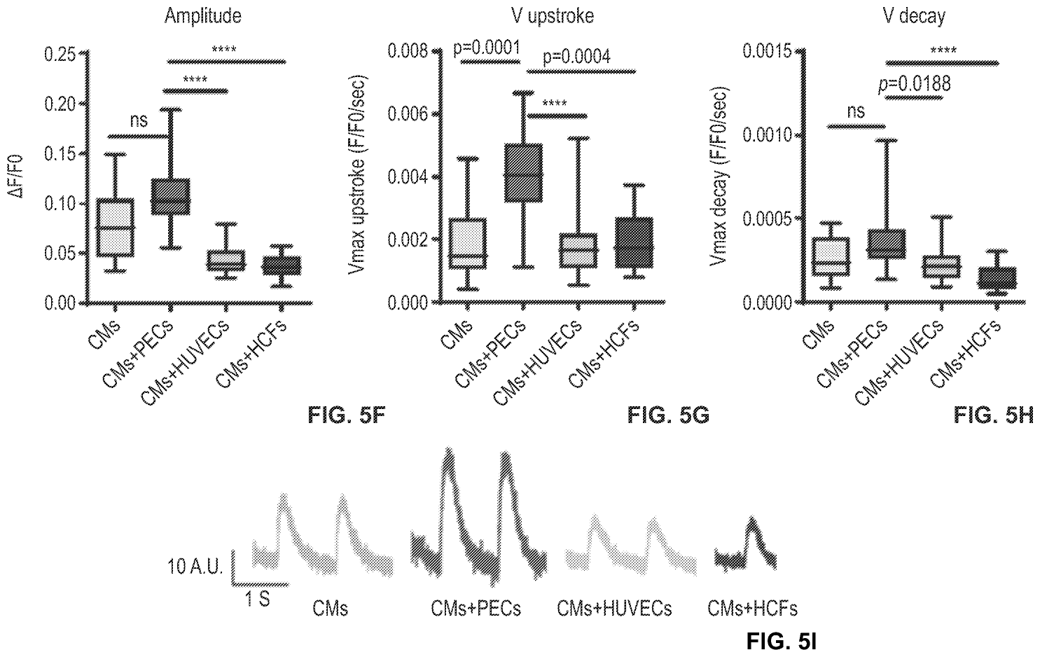

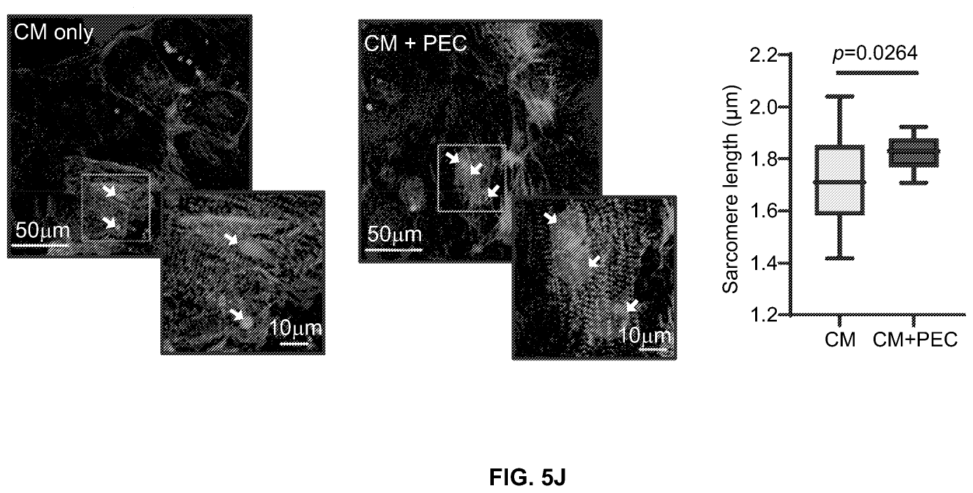

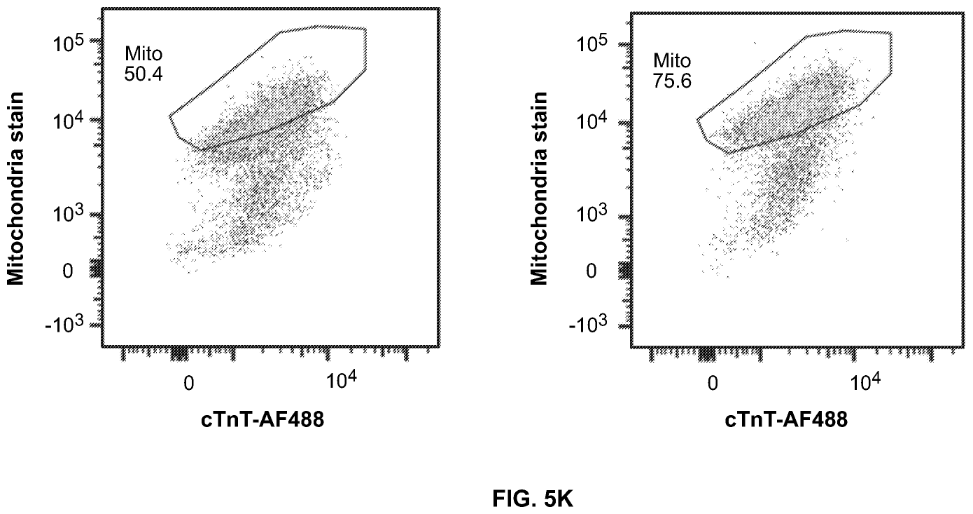

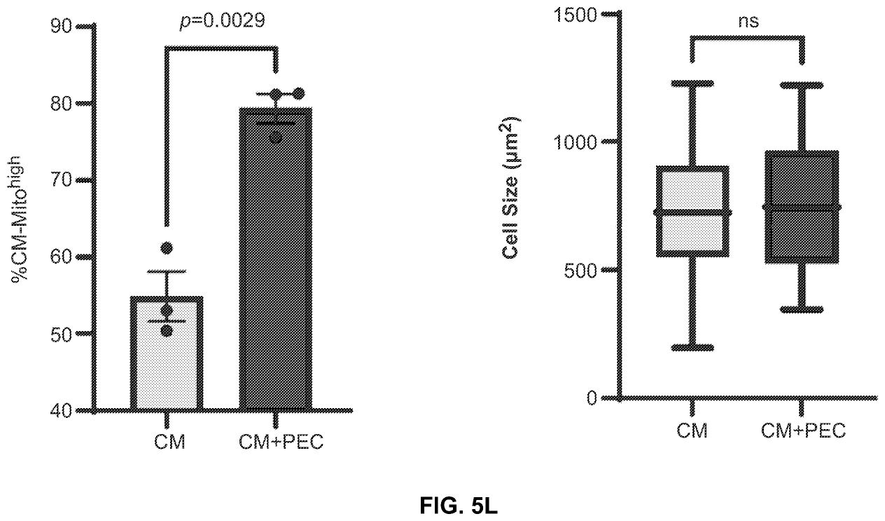

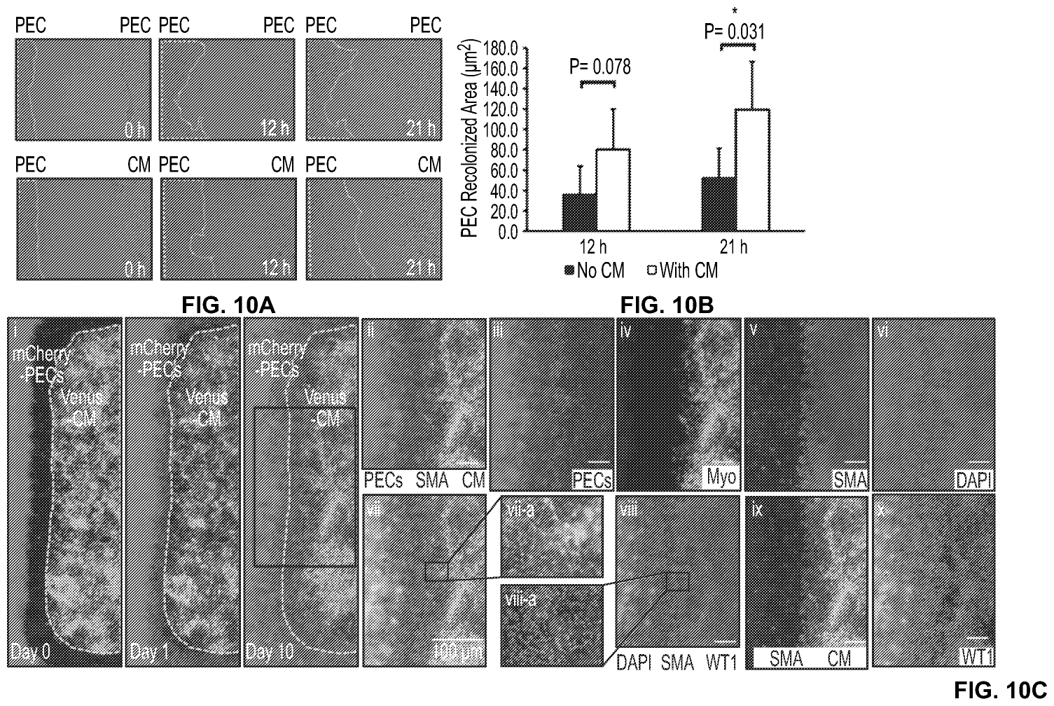

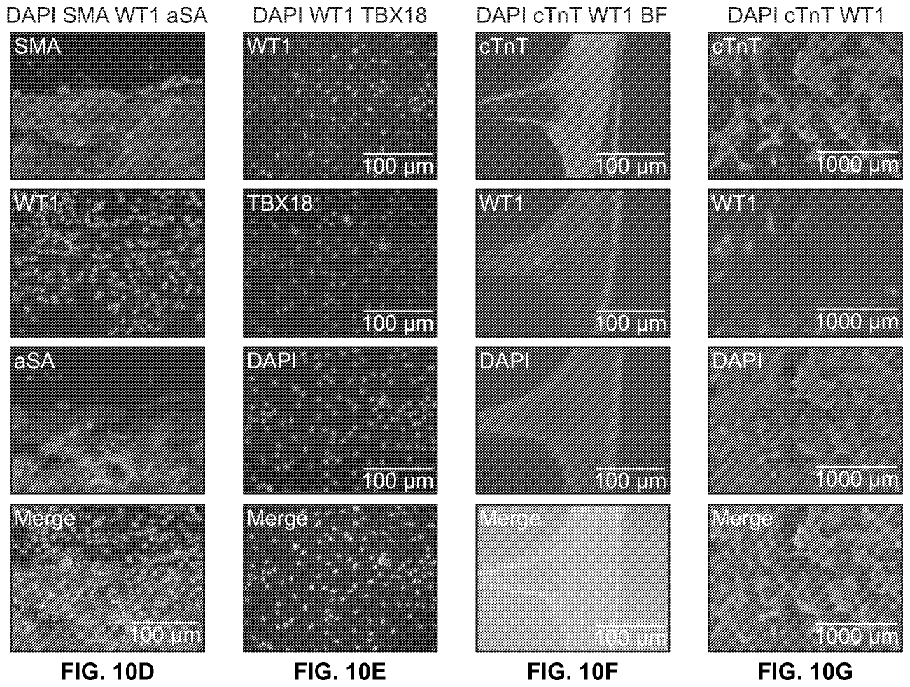

Cell coverage and 2D-culture aggregate height analysis. Cell Coverage: To assess cell coverage, Venus-tagged CMs (vCMs) were seeded either alone (250,000 cells/well in a 24-well plate) or in co-culture with other cell types including PECs, HUVECs, and HCFs (250,000 vCMs, with 250,000 cells of the co-cultured cell-type). Coverage was measured by isolating the green, fluorescent vCM signal from each ROI, thresholding all images to the same degree to subtract background, creating binary images, and then measuring surface-area coverage of signal versus total ROI area. Images were recorded using a Nikon Eclipse TE200 microscope (Nikon, Melville, NY). Image post-processing and surface-area coverage measurements were done using ImageJ software. In analyses of all groups at day 8 and 16, cell coverage measurements were taken from broad-field areas (7.6 mm²) in 3 independent experiments (FIGS. 4E and 5C). Data presented as mean±SD. 2D-culture Aggregate Height: For height analysis, cell coverage experiments were analyzed at day 8 for all groups (FIG. 10E) and at day 16 for CMs-alone vs. PEC-CMs (FIG. 4E). Venus-tagged CM (vCM) aggregates were initially identified using fluorescent microscopy, using a Nikon Eclipse TE200 microscope. Microscope setting were changed for light microscopy, and then z-plane analysis was performed to carefully dial into high-resolution views of the top and bottom of the 2D-culture aggregates. Z-plane distance between the microscope lens and sample was recorded at both top/bottom focal planes using Nikon NIS-Elements Advanced Research software (measured in μm), and aggregate heights were determined by taking the difference of these positions. N=12 for CM-alone and PEC-CMs at days 8 and 16 (FIG. 4C), as 4 measurements in each of 3 independent experiments (data presented as mean±SD). For expanded analysis of all groups at day 8 (FIG. 10E), n=15 for all groups as 5 measurements in each of 3 independent experiments (data presented as mean±SD).

For all groups at day 8 in F (across 3 independent experiments), n=15 for both groups at day 16 (across 3 independent experiments). Data presented as mean±SD.

Contractility analysis. Strain Measurement. We compared CM-only culture (1,500,000 CMs/well in 6-well plates) to PEC-CM co-culture (900,000 CMs/well with 1,250,000 PECs/well in 6-well plates), HUVEC-CM co-culture 900,000 CMs/well with 625,000 HUVECs/well in 6-well plates), and HCF (900,000 CMs/well with 625,000 HCF/well in 6-well plates co-culture. Experimental groups were seeded onto collagen gels consisting of 2.0 mg/mL collagen (collagen type I, derived from rat tails), supplemented with 0.9 mg/mL Matrigel. At 14 days, we acquired high-speed videos of beating areas at 10× magnification using a Nikon Eclipse TE200 microscope, under pacing conditions (20 V, 0.7 Hz, 6.0 ms) using a C-Pace cell culture stimulator (IonOptix LLC, Milton, MA). Videos were acquired with a 1280×1024-pixel resolution at a frame rate of 20 fps for 20 seconds, and then deconstructed to image-stacks. Strain of contracting areas was evaluated on successive images using a high spatial resolution sub-pixel algorithm called high density mapping (HDM) (37) We have previously used the HDM method to assess contractile strain in cardiac applications in vitro and in vivo (38, 39) Multiple contractions were analyzed per area (>3), and several areas were analyzed (n=7 for each group), and averaged to determine area-strain (dA/A). Atomic Force Microscopy (AFM): After strain measurements, cell-seeded gel samples (CM only, CM-HUVEC, CM-PEC, and CM-HCF) were cut to 1 cm by 1 cm squares and mounted on a petri dish and then submerged in cell medium to allow for AFM sampling. An Asylum Research MFP-3D-BIO AFM was used to image cell seeded gels and measure the elastic modulus. Sharp conical cantilever tips made of silicon nitride were used that had a nominal spring constant of 0.06 N/m (DNP; Bruker Nano Inc.) For each sample (n=6 for each group), three force maps were taken at separate locations; each force map consisted of 16 individual force curves (although 2 of the maps in the CMPEC samples only had 8 force curves taken due to a lack of height in the sample). Each force curve was fit to a Hertzian model over a 400 nm indentation range to find the resulting Young's modulus value (E). Contractility: Using the formula F=(E)(dA/A)(Area), we calculated contractility (Force/Area) of the CMs in each group based on our measurements for strain and modulus.

Calcium transients. We compared CM-only culture (1,500,000 CMs/well in 6-well plates) to PEC-CM co-culture (900,000 CMs/well with 1,250,000 PECs/well in 6-well plates), HUVEC-CM co-culture 900,000 CMs/well with 625,000 HUVECs/well in 6-well plates), and HCF (900,000 CMs/well with 625,000 HCF/well in 6-well plates co-culture. At day 14, intracellular calcium [Ca2+]i transients were studied with ratiometric intracellular calcium indicator Fluo-3 acetoxymethyl ester (Fluo-3 AM) at 37° C. Cells were loaded with ˜4.4 μmol/L Fluo-3 AM (Invitrogen, USA) in normal Tyrode's solution (NaCl 136 mM, KCl 5.4 mM, MgCl2 1 mM, CaCl2) 1.8 mM, NaH2PO4 0.33 mM, HEPES 5 mM and dextrose 10 mM; pH 7.35 with NaOH) in the presence of 0.02% Pluronic F-127 for 10 minutes at room temperature, followed by a 30 minutes washout of Fluo-3 AM for its de-esterification. Fluo-3 fluorescence was obtained with a FITC filter set (excitation: HQ480, mirror: Q505LP, emission: HQ535/50 m; Chroma) and an X-Cite exact mercury arc lamp (Luman Dynamics) with a 50% output for illumination. Fluorescent images were recorded with a Nikon Eclipse Ti-U inverted microscope (Nikon Instruments Inc., Melville, in [Ca2+]i NY, USA), a NeuroCCDSM camera (RedShirtImaging), and the Neuroplex software (RedShirtImaging, Decatur, GA, USA). Calcium transients were analyzed with Neuroplex and Clampfit 9.2 (Molecular Devices Inc., Sunnyvale, CA, USA). Multiple transient curves were analyzed per area (3-4), and several areas were analyzed in each condition (≥15). Fluo-3 AM calcium transient tracings were presented as ΔF/F0, F0 is the baseline fluo-3 fluorescence at resting state. Cyclic calcium transients were analyzed to determine amplitude, transient upstroke velocity, and transient decay velocity with a monoexponential fit. Data presented as mean SEM.

CM-sphere, PEC-sphere, and aggregate formation. To generate spontaneously beating CM-spheres from hiPSCs in suspension culture.79 Briefly, hiPSCs were cultured in spinner flasks at 45 rpm for 24 h to form spheres, and then cardiac differentiation was achieved by 24 h treatment with 12 μM CHIR followed by 48 h treatment with 12 μM IWP4, resulting in spontaneously beating CM-spheres by day 10. To generate PEC-spheres, freshly differentiated PECs at day 7 were dissociated into single cells and suspension-cultured in RPMI supplemented with insulin-containing B27 and PECM at 1:1 ratio in a spinner flask using CELLSPIN system (Integra Biosciences AG, Switzerland), and spun at 45 rpm at 37° C. PEC spheres were mostly visible after 24 h. To create spontaneously beating cardiac aggregates, CM-spheres and PEC-spheres were sampled (10-20 spheres per group) and cultured in 1 well of a 24-well ultra-low attachment polystyrene plate (Corning Incorporated, Kennebunk, ME). Wells were fed every 2 days with insulin supplemented B27 in RPMI and PECM at 1:1 ratio. Rotation culture was the ultra-low attachment polystyrene culture plates with CM spheres and PEC-spheres that were placed on GyroMini Nutating Mixer (Labnet) ran at a fixed rotation speed of 20 rpm under standard cell culture conditions. Structures self-assembled over 48 h, with PECs incorporating with CM-spheres. Calcium transient and histology were assessed after 10 days in culture.

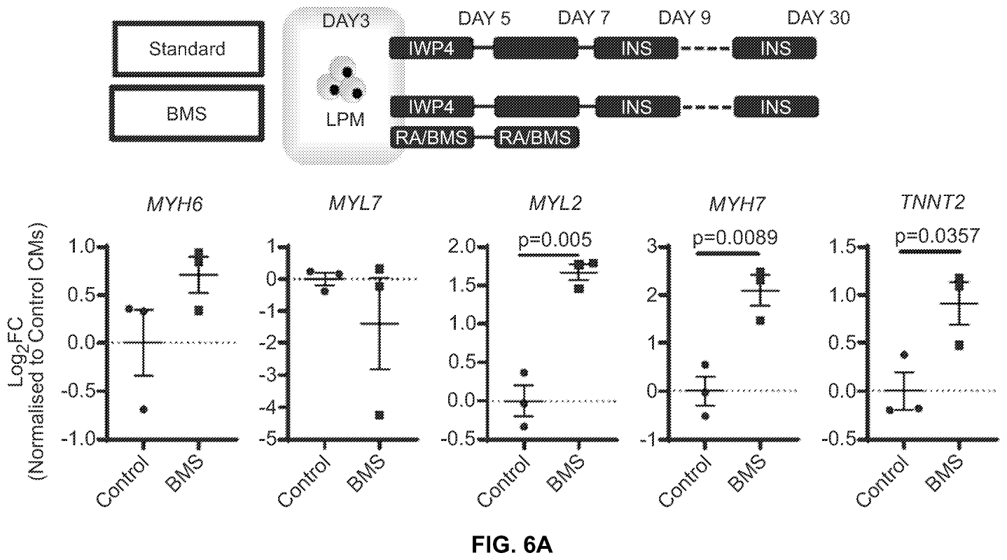

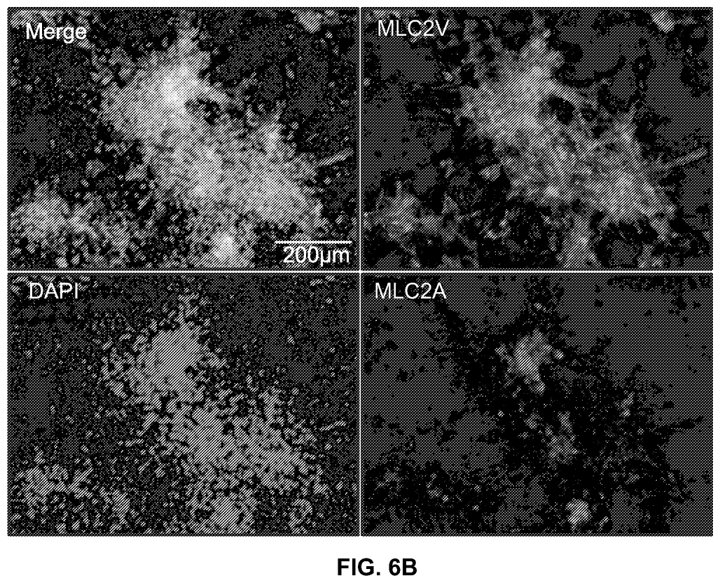

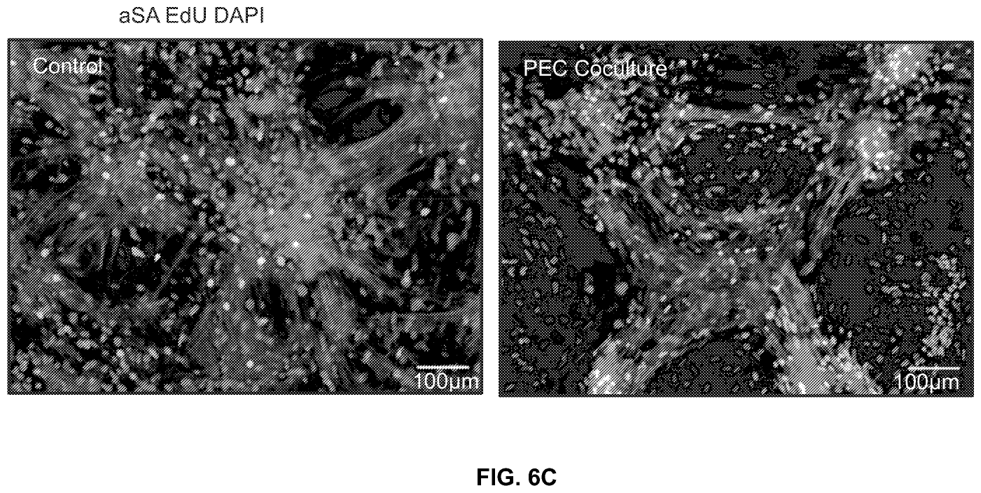

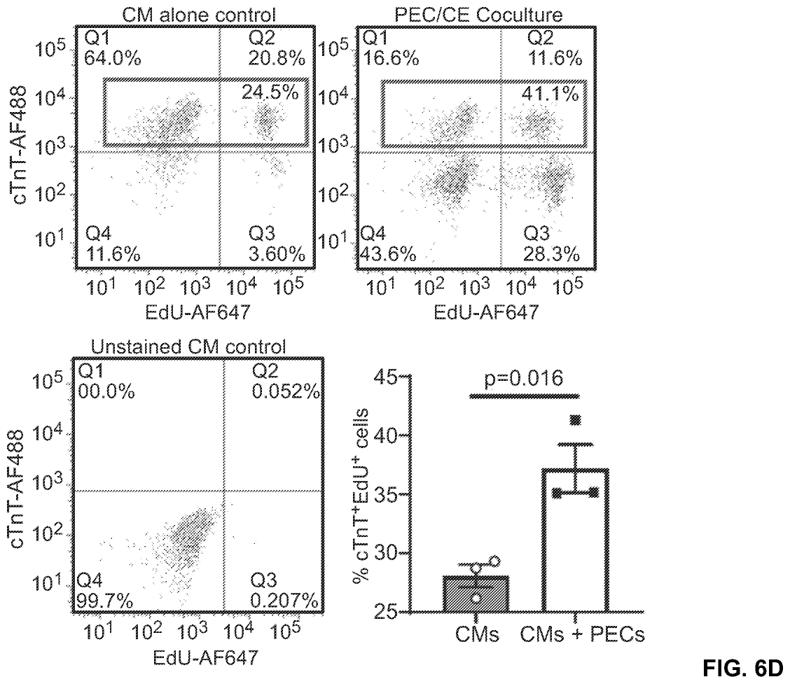

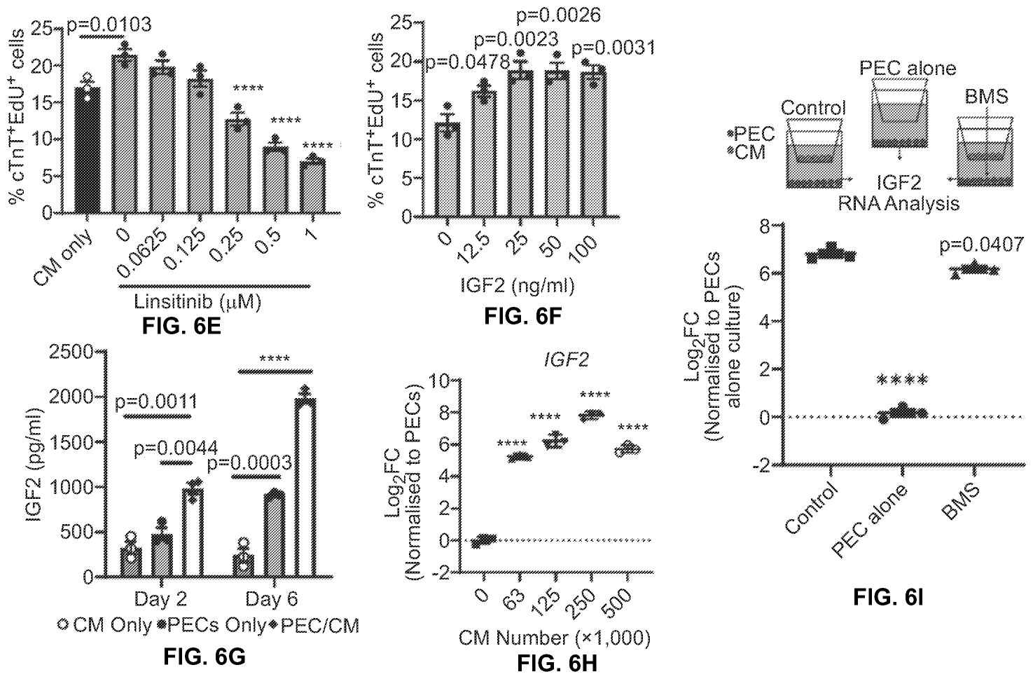

Measurement of sphere density, size, structure: A total of PEC and CM spheres were measured from day 1 of its formation after culturing in spinner flask culture (herein indicated as day-7 in FIG. 6B), and a total PEC spheres of 1303 and total of CM sphere of 1316, were included over the course of 7 days to calculate structures/spheres prior to co-culturing with CM spheres, using FIJI/ImageJ software under 10× magnification (n=3 for day −7 −5. −3 and −1). To calculate percent of spheres with structure and the number of structure/spheres, we examined a total of 10 PEC spheres at day −7, 13 at day −5, 23 at day −3 and 30 at day −1. For CM spheres, we examined a total of 30 spheres at each timepoint from day −7 to day −1. The average area of structure in PEC spheres were derived from 16 measured structures at day −7, 35 at day −5, 137 at day −3 and 82 at day −1. Whereas the average area of structure in CM spheres were derived from 70 measured structures at day −7, 115 at day −5, 125 at day −3 and 69 at day −1. All readings were measured using FIJI/ImageJ software (Image J 1.53c, NIH) based on images taken at each timepoint at 10× magnification under Nikon Eclipse TE200 light microscope.

At day 15, PEC spheres, CM spheres and PEC/CM spheres were measured for sphere density (total nuclei per mm²), average sphere area in mm² (n=3, with total measured PEC spheres=10, CM spheres=10, PEC/CM spheres=3), structure per sphere (n=3, with total measured PEC spheres=10, CM spheres=10 and PEC/CM spheres=8), and structure size/spheres size (n=3, with total measured structure of 25 for PEC spheres, n=4 with total measured structure of 11 for CM spheres, and n=3 with a total measured structure of 13 for PEC/CM spheres). In additions, the number of structures per PEC/CM sphere and the average area of structure were measured at day 5 (n=10, with a total of 25 measured structures) and day 15 (n=3, with a total of 13 measured structures). All data was presented in mean±SEM.

TUNEL stain: TUNEL stain was performed according to manufacturer's protocol (DeadEnd™ Fluorometric TUNEL System, Promega Corporation, Madison, WI)). Briefly, Spheres were collected and fixed in 4% paraformaldehyde, embedded in Histogel Specimen Processing Gel (Thermofisher Scientific, Waltham, MA) prior to embedding in paraffin. Spheres were sectioned at 5 microns each slice, deparaffinized using fresh xylene, rehydrated with 100%, 95%, 85%, 70%, 50% graded ethanol and washed with 0.85% NaCl at room temperature prior to fixing with 4% formaldehyde solution. Then the sphere sections were washed with PBS and pretreated with 20 μg/ml Proteinase K solution for 10 min, after which were washed with PBS prior to labelling with rTdT incubation better solution consisted of equilibrium buffer, nucleotide mix and rTdT enzyme as provided by the manufacturer. All sphere sections were counterstained with DAPI to visualize nuclei and apoptotic cells with fluorescein-12-dUTP fluorescein (green) were identified using Nikon Eclipse TE200 fluorescence microscope.

Hematoxylin and Eosin Stain: Paraffin embedded tissue sections were deparaffinized in oven at 56 C for 15 min and rehydrated with HistoClear solution (National Diagnostics, Atlanta, GA) twice for 5 min, twice with 100% Ethanol (Fisher Scientific, Fair Lawn, NJ) for 3 min, 95% ethanol for 3 min and wash with distilled water for 2 min prior to staining with Hematoxylin (Vector laboratories, Burlingame, CA). Then, the slides were rinsed with distilled water for 2 min and counter-stained with Eosin (Vector laboratories, Burlingame, CA) with 15 dips, after which were rinsed with 95% ethanol and distilled water. Dehydration of slides were performed with submerging the slides for 3 min in 100% ethanol, and twice with HistoClear for 3 min. Sections were mounted with Permount solution, covered with coverslips for microscopic examination.

Statistical analysis. All box and whisker's plot presented 25th, 50th and 75th percentiles minima and maxima. Dotplots for gene expression data and bar graphs are presented in mean±SEM. All data were representation of three independent replicates (unless stated otherwise) and expressed in mean±standard error of mean. Difference between groups was analyzed using unpaired two-tailed student T-test (for 2 groups) or one-way ANOVA with Dunnett (for comparing mean of a defined control) or Tukey post-hoc test for multiple comparisons, and it was considered statistically significant when p<0.05. Unpaired T-test with Welch's correction was used for groups with unequal variance, which was tested using Shapiro-Wilk test. All analyses were performed using GraphPad Prism 8.0.2.

TABLE I
List of Primary antibodies used in the studies

| | | Application, |
|---|---|---|
| Primary antibody | Source/Catalog number | dilution |
| Mouse anti-ZO1 | Thermo Fisher Scientific | ICC, 1:200 |
| | Cat# MA339100A488, | |
| | RRID: AB_2633345 | |
| Mouse anti TBX18 | R and D Systems Cat# | ICC, 1:200 |
| | MAB63371, | |
| | RRID: AB_10892533 | |
| Mouse anti-CD31 | Agilent Cat# M0823, | ICC, 1:200 |
| | RRID: AB_2114471 | |
| Mouse anti-smooth | Abcam Cat# ab7817, | ICC, 1:500 |
| muscle actin | RRID: AB_262054 | |
| Mouse anti-cardiac | Abcam Cat# ab8295, | ICC/FC, |
| troponin T | RRID: AB_306445 | 1:500 |
| Mouse anti sarcomeric | Sigma-Aldrich Cat# A7811, | ICC, 1:250 |
| α-actinin | RRID: AB_476766 | |
| Rabbit anti-ACTN2 | Creative Diagnostics, | ICC, 1:200 |
| monoclonal antibody | DCABH-9438 | |
| | RRID: AB_2482431 | |
| Mouse anti MLC2A | Abcam Cat# ab68086, | ICC, 1:200 |
| | RRID: AB_1140497 | |
| Rabbit anti-WT1 | Abcam Cat# ab89901, | ICC/FC, |
| | RRID: AB_2043201 | 1:250 |
| Rabbit mAb to TCF21 | Abcam Cat#ab182134 | ICC, 1:200 |
| | RRID: AB_2889038 | |
| Rabbit anti-RFP | Abcam Cat# ab62341, | ICC, 1:200) |
| | RRID: AB_945213 | |
| Rabbit anti-GFP | Abcam Cat# ab290, | (1:200) |
| | RRID: AB_303395 | |
| Rabbit anti-VE-Cadherin | Abcam Cat# ab33168, | (1:200) |
| | RRID: AB_870662 | |
| Rabbit anti-Calponin | Abcam Cat# ab46794, | (1:200) |
| | RRID: AB_2291941 | |
| Rabbit anti-myl2 | Abcam Cat# 2917-1, | (1:200) |
| | RRID: AB_2266753 | |
| PE-mouse anti-KDR | BD Biosciences Cat# 560872, | (FC, 1:50) |
| | RRID: AB_10564096 | |
| Alexa Fluor ® 647 anti- | BioLegend Cat# 147308, | (1:200) |
| mouse/human CD324 (E- | RRID: AB_2563955 | |
| Cadherin) Antibody | | |
| Alexa Fluor ® 647 Mouse | BD Biosciences Cat# 561567, | (FC, 1:20) |
| Anti-Human CD144 Clone | RRID: AB_10712766 | |
| 55-7H1 (RUO) | | |

### Example 1: Rapid Derivation of Pre-Epicardial Cells from Lateral Plate Mesoderm Using BMP4, VEGF, and RA

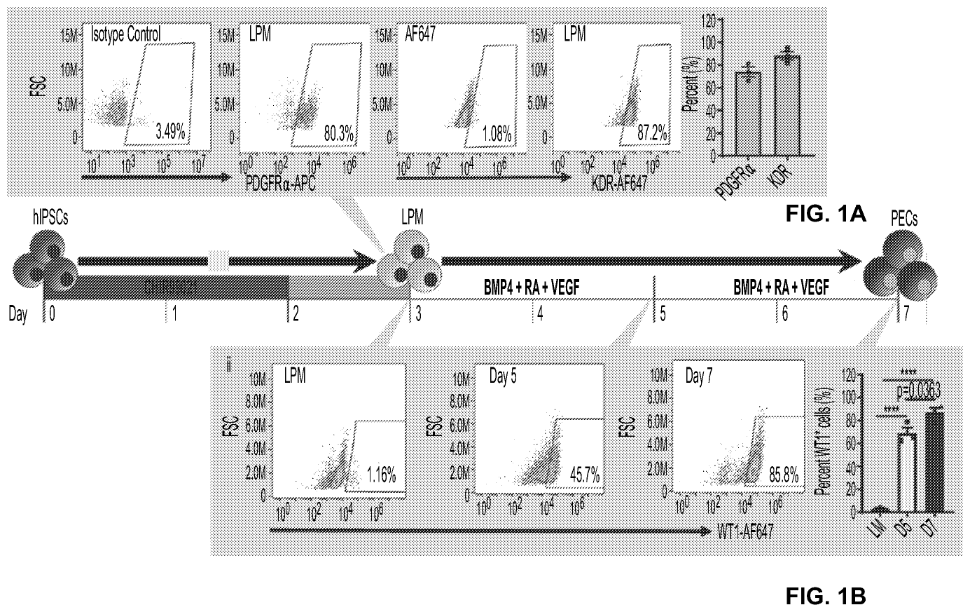

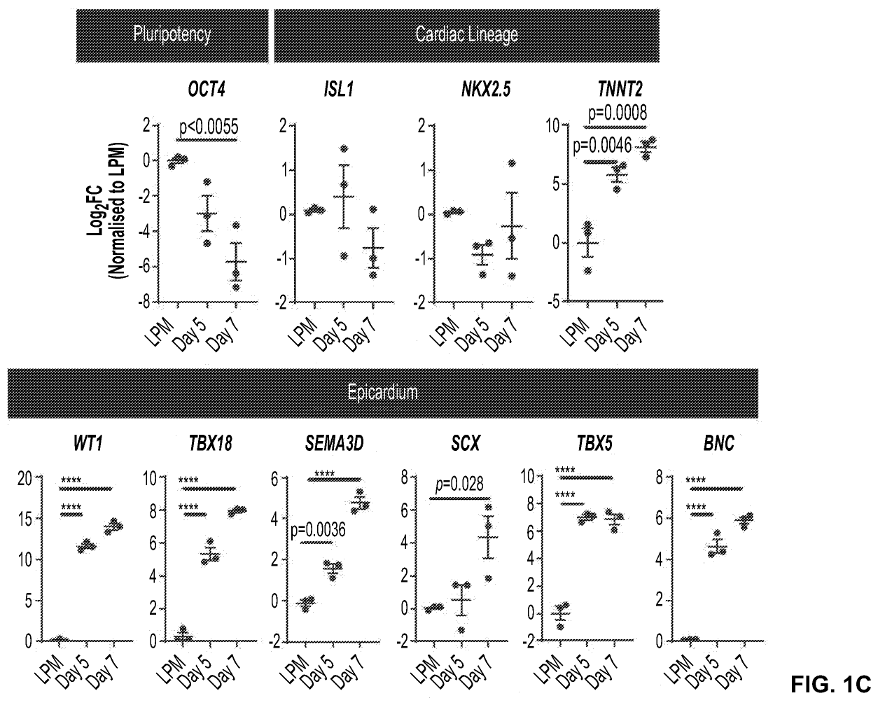

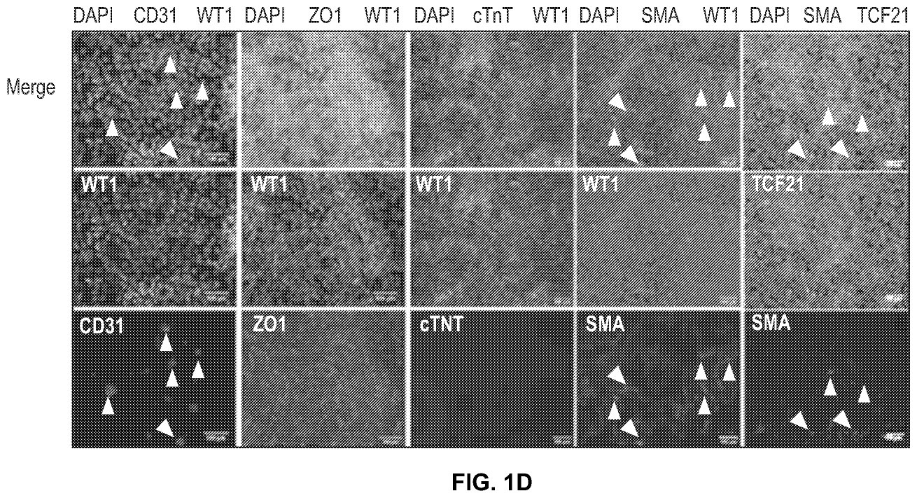

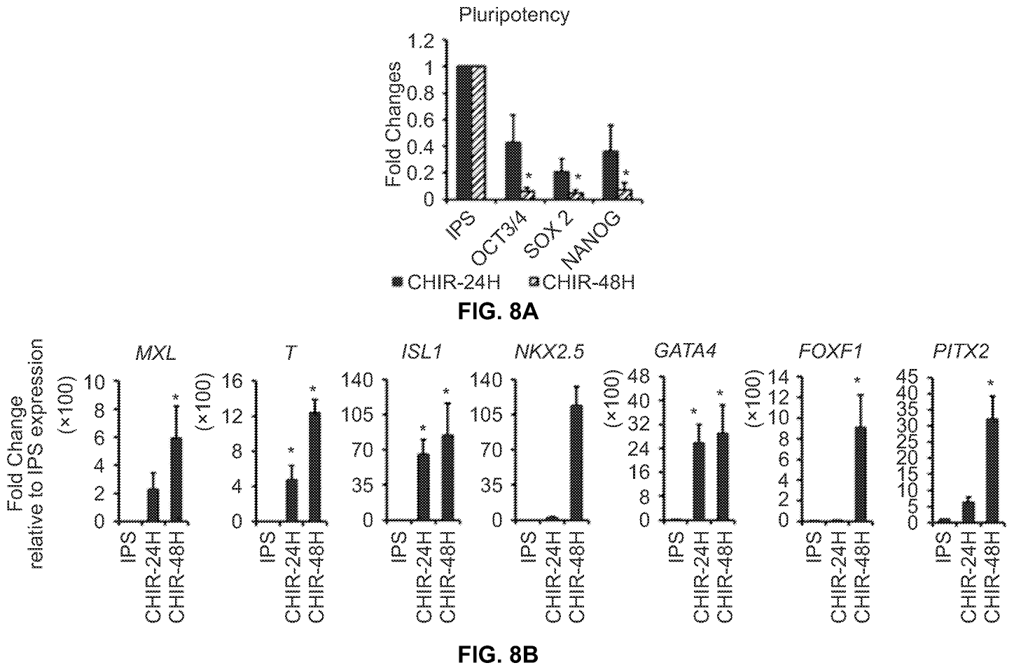

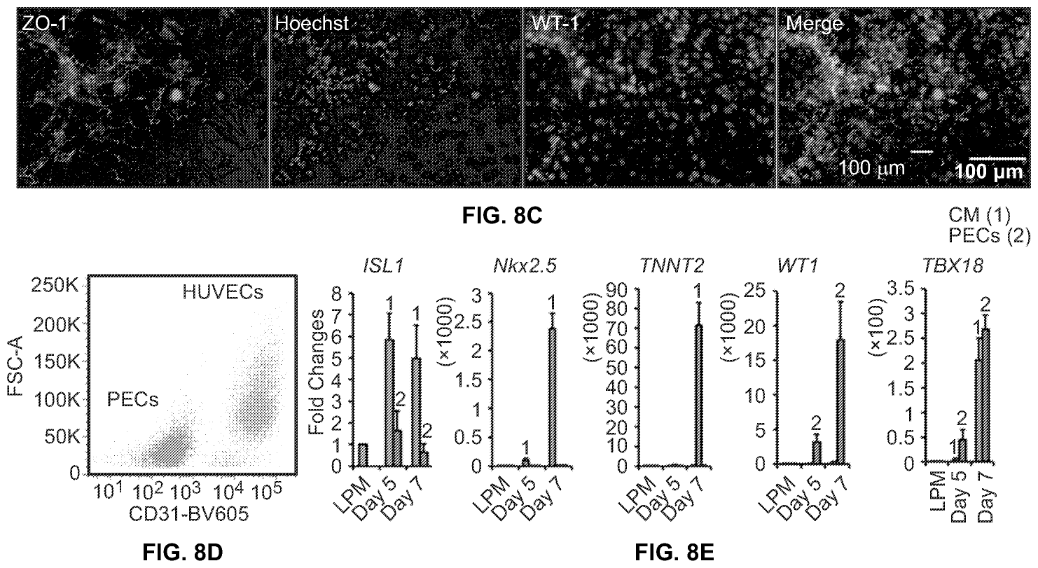

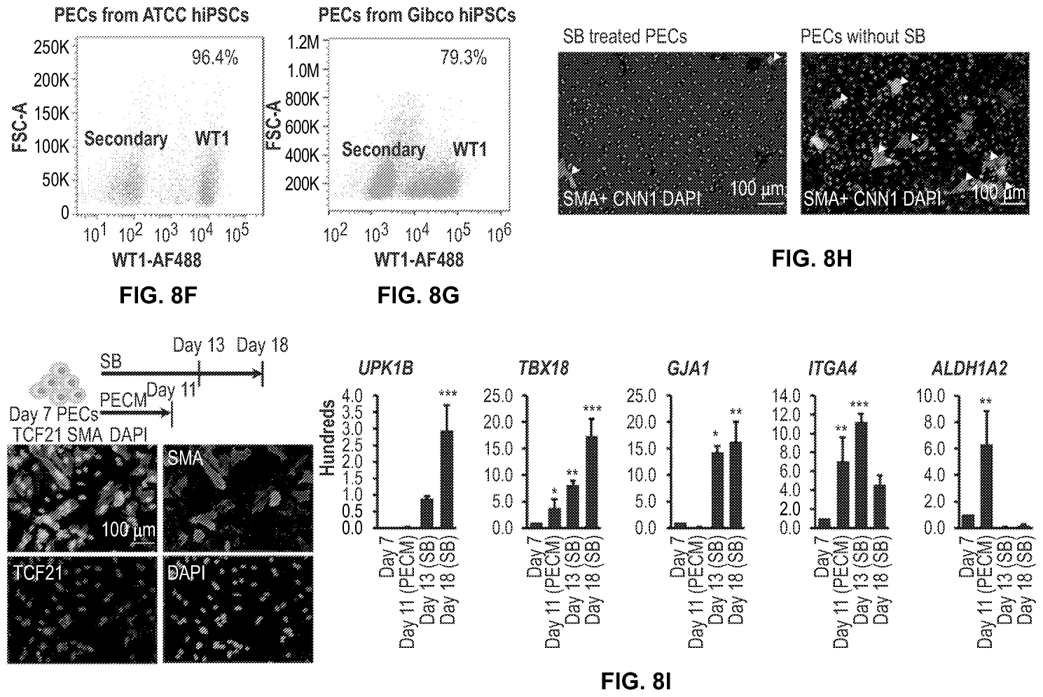

PECs and CMs have been shown to share a similar pool of cardiac precursors derived from LPM (17, 18), and LPM can be derived from hiPSC with prolonged CHIR treatment (40). This method was adopted to generate LPM progenitors, most of which expressed platelet-derived growth factor receptor-alpha (PDGFRA, 73.7±4.3%) and/or vascular endothelial growth factor receptor-2 (KDR, 88.0±3.6%; FIG. 1A) at day 3. The 48 h CHIR treatment also demonstrated significant downregulation of pluripotency markers (i.e. OCT3/4, SOX2, NANOG), as well as significant upregulation of primitive streak markers (i.e. MXL and T), lateral plate mesoderm marker (i.e. PITX2 and FOXF1), and cardiac progenitor markers (i.e. GATA4, ISL1, and NKX2.5; FIGS. 8A-8B). BMP4, VEGF, and RA signaling (collectively abbreviated as BVR) are key biochemical factors that drive epicardial differentiation (41). The LPM was induced with BVR for additional 96 hours, and 86.8±4.1% WT1 cells at day 7 (n=3) were successfully derived within the same monolayer LPM culture without cell splitting or embryoid body formation (FIG. 1). WT1 cells also demonstrated significant increases in WT1, TBX18, SEMA3D, TBX5, BNC (p<0.0001) and SCX (p=0.028) transcript expression, the known epicardial markers (FIG. 1C). Immunofluorescent (IF) staining showed high-level expression of WT1⁺, TCF21⁺, and ZO1⁺ (FIG. 1c), with few cells exhibiting endothelial marker CD31V (FIGS. 1D and 8C) or smooth muscle marker SMA⁺ (FIG. 1D). No cTnT⁺ cells were detected in any PEC differentiations, despite an upregulation of TNNT2 gene in PECs. Comparing with differentiating CM, TNNT2 gene was 235× lower in PECs (FIG. 8D). To demonstrate the reproducibility of the differentiation as shown above, this protocol was tested and successfully reproduced using two additional commercial hiPSC lines (FIGS. 8E-8F).

### Example 2: RNA Sequencing Revealed PEC Profile Relatives to Known Epicardium Cells

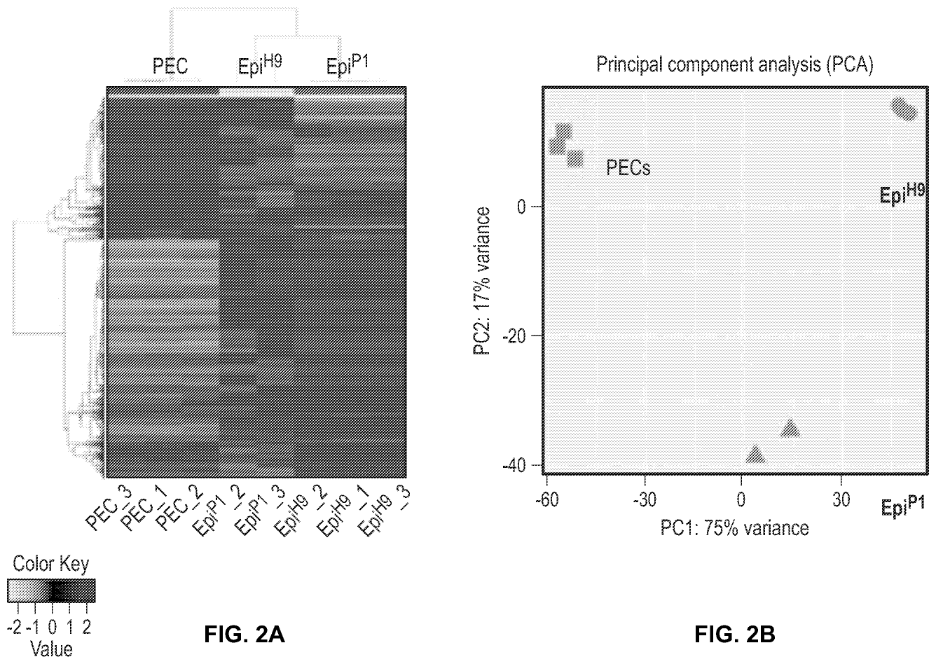

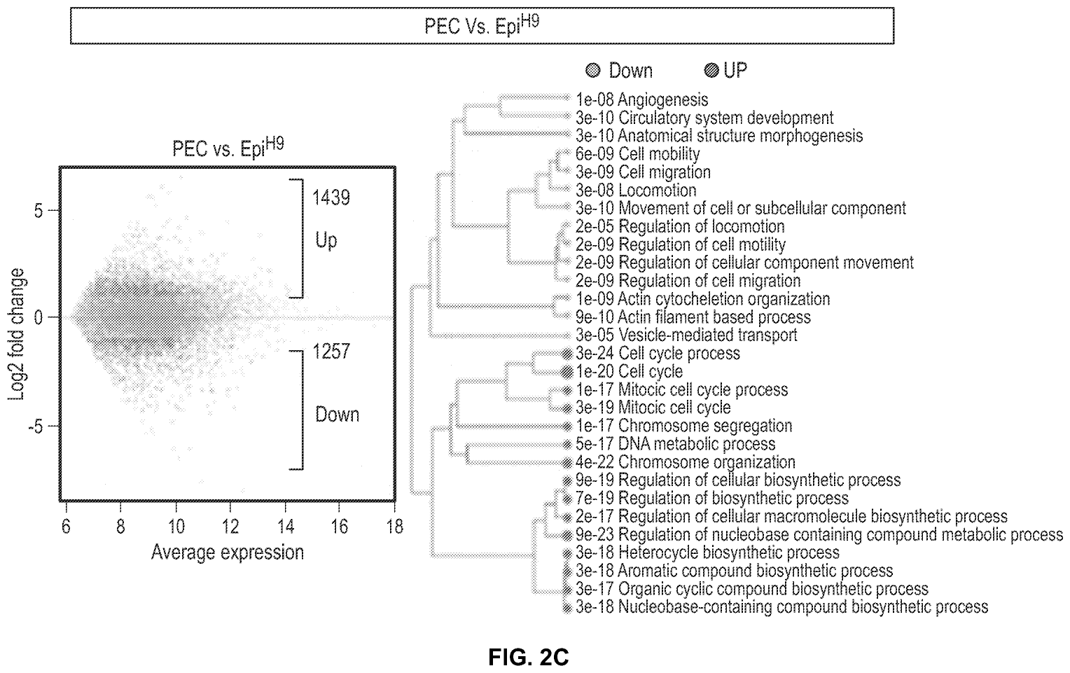

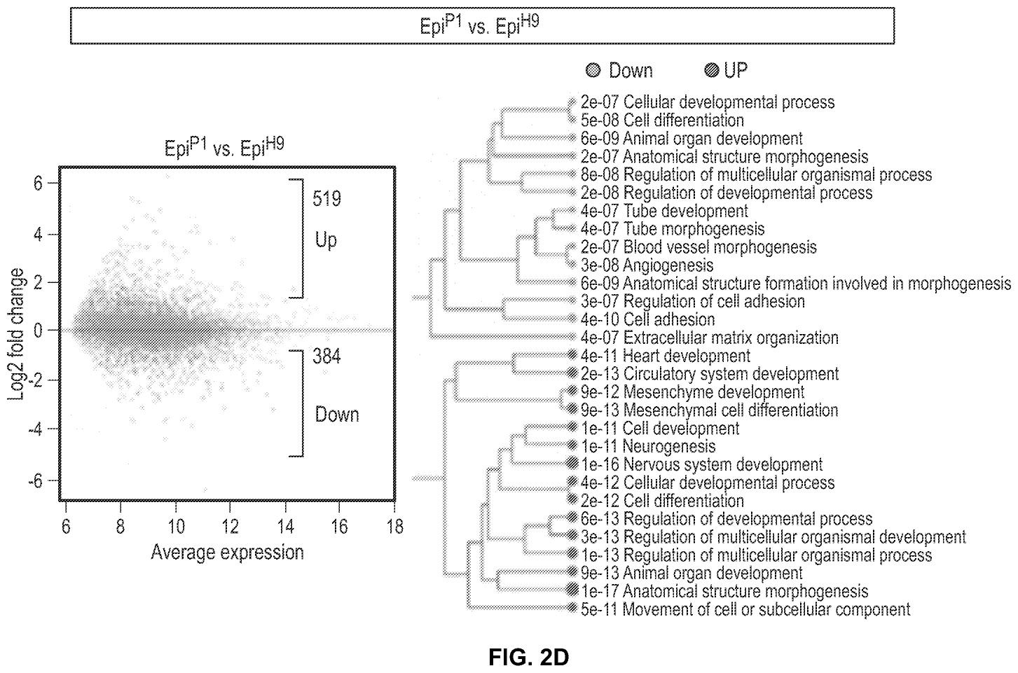

An unsupervised hierarchical gene expression clustering analysis was performed by comparing transcriptomic profiles between PECs, expanded PECs at passage 1 (Epi^{P1}), and H9-derived epicardial cells (Epi^{H9}) from the Palecek group (FIG. 2)(20). Clustering of the 2000 most variable genes showed distinct gene expression profiles among PEC, Epi^{P1} and Epi^{H9} cells (FIG. 2A). The Epi^{P1} population (derived from PECs) was clustered closer to the Epi^{H9} population (Pearson's correlation coefficient, r=0.88-0.91) than the PECs (r=0.75-0.78; FIG. 2A). However, the principal component analysis determined that PECs were clustered closer to Epi^{P1} than Epi^{H9} (FIG. 2B). Nonetheless, many of the upregulated differentially expressed genes (DEGs) in PECs were found to be downregulated in Epi^{P1} (FIG. 2C). The total DEGs was reduced from 2,696 in the PECs vs. Epi^{H9} comparison (FIG. 2C) to 903 in Epi^{P1} vs. Epi^{H9} comparison (FIG. 2D). Of those, 607 of the DEGs were consistently present in both stages. Gene enrichment analysis based on Gene Ontology (GO) for biological processes demonstrated that upregulated DEGs of PECs vs. Epi^{H9} were highly enriched for genes involved in cell cycle whereas the downregulated DEGs were enriched for angiogenesis, circulatory system development, and cell motility or migration (FIG. 2C). This is in contrary to Epi^{P1} vs. Epi^{H9} of which the DEGs involved in the heart, mesenchyme, circulatory and nervous system development were upregulated, while the downregulated DEGs were enriched for angiogenesis, cell adhesion/regulation, and blood vessel morphogenesis similar to that of PECs vs. Epi^{H9} (FIG. 2D).

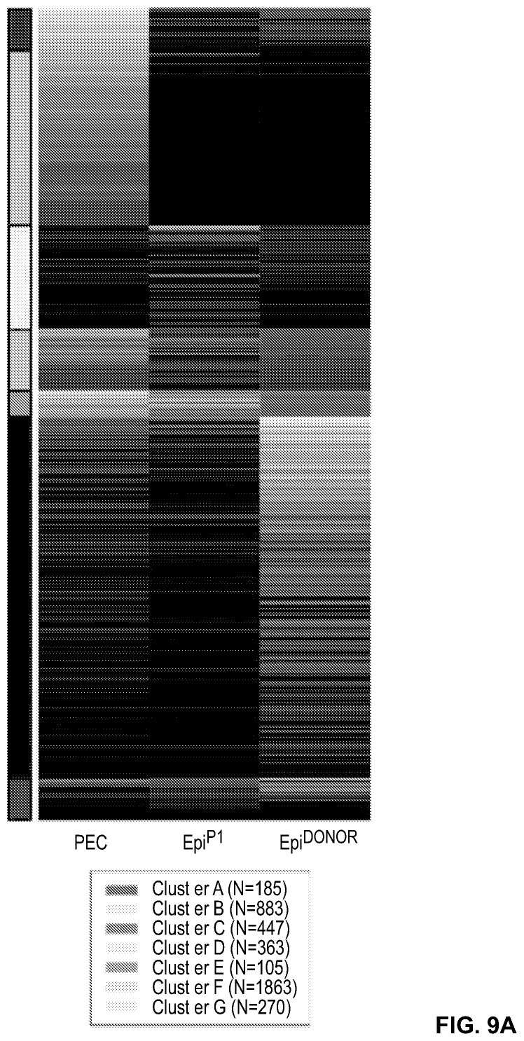

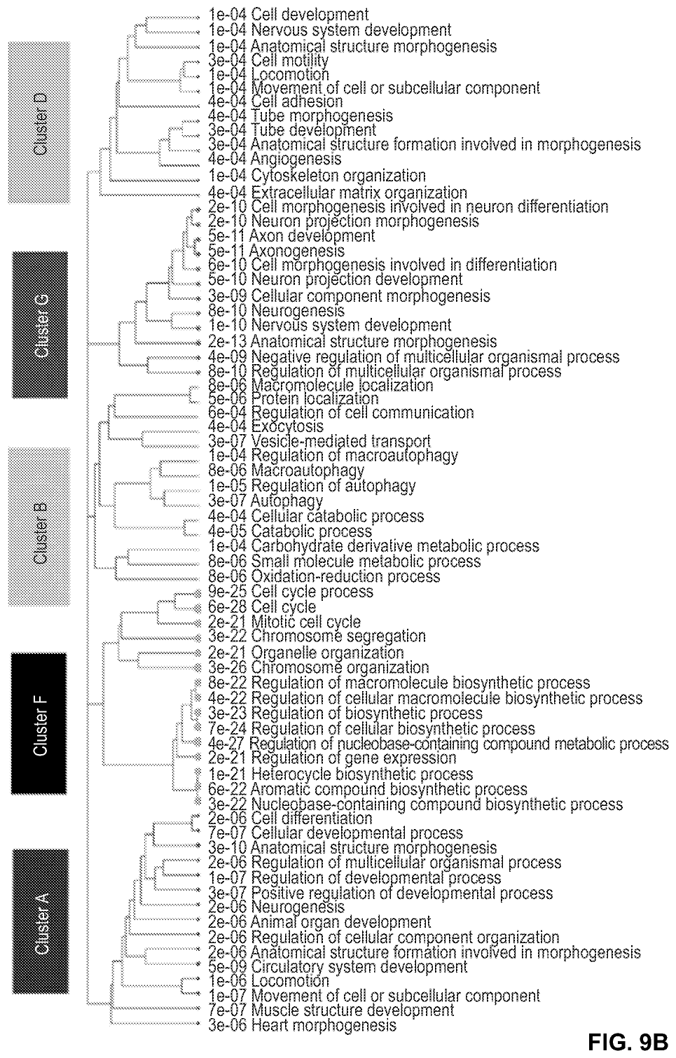

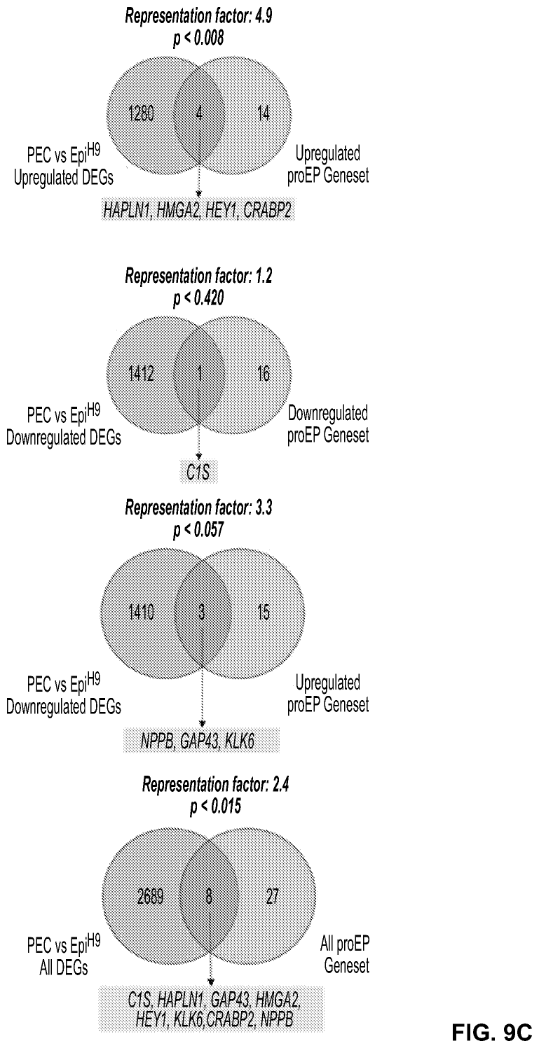

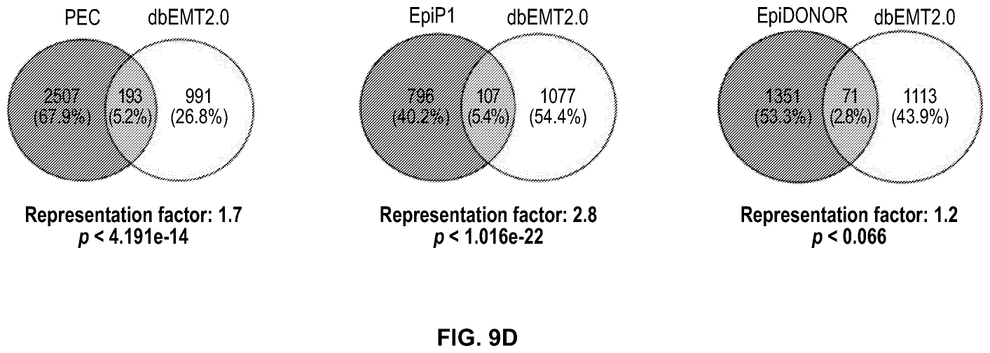

To further characterize the differentiation stage of PECs relative to known epicardial lines, the dataset of epicardial transcriptomes from hiPSC-derived (Epi^{SC} from 19-9-11 and 19-9-97 lines), hESC-derived (Epi^{SC} from H9 and ES03 lines), and donor-derived (Epi^{DONOR} from Donor9635, Donor9634, Donor9633 and Donor9605) were included in the analyses³². The log₂ fold-change of DEGs obtained from PECs vs. Epi^{H9}, Epi^{P1} vs. Epi^{H9}, and Epi^{DONOR} vs. Epi^{SC} were employed for comparisons to avoid batch effect between the two independent studies. K-means clustering was performed on all the DEGs and functionally group into 7 clusters (A to G) based on enriched gene ontology terms for biological processes (FIGS. 9A-9B). Consistently, the DEGs that were enriched for ontologies related to cell cycle and mitosis (Cluster F) were only found to be upregulated in PECs as compared to Epi^{P1} and Epi^{DONOR} Whereas the upregulated DEGs in Epi^{P1} is similar to Epi^{DONOR}, which were enriched for ontologies involving cell differentiation, neurogenesis, circulatory and heart morphogenesis (Cluster A), as well as processes related to cell signaling, metabolism, and communications (Cluster B). Despite the common ontologies, upregulated DEGs from Epi^{P1} were uniquely enriched for nervous system development (Cluster G), whereas Epi^{DONOR} uniquely enriched for ontologies related to cell motility and angiogenesis (Cluster D).

Enrichment analysis was then performed using the exact hypergeometric probability test on the DEGs lists for PEC and Epi^{P1} with the human proepicardial (hProEP) geneset (35 genes) extracted from the 2019 Cui et al. study (42). The analysis indicated that both PEC and Epi^{P1} DEGs were 2.4 (p<0.015) and 4.5 (p<0.005) times more overlaps with the hProEP geneset than expected when compared to the background (28,397 genes), respectively (FIG. 9C). Comparing the DEGs to gene set for the epithelial to mesenchymal transition geneset from dbEMT 2.0 previously curated by Zhao et al. (2014, 2019; FIG. 9D) (43, 44), DEGs from PECs and Epi^{P1} were 1.7 (p<4.178e-14) and 2.8 (p<1.013e-22) times more overlapped with the dbEMT 2.0 geneset, respectively. However, DEGs from Epi^{DONOR} did not overlap significantly with the geneset.

### Example 3: Differentiated PECs are Capable of Epithelial-Mesenchymal Transition

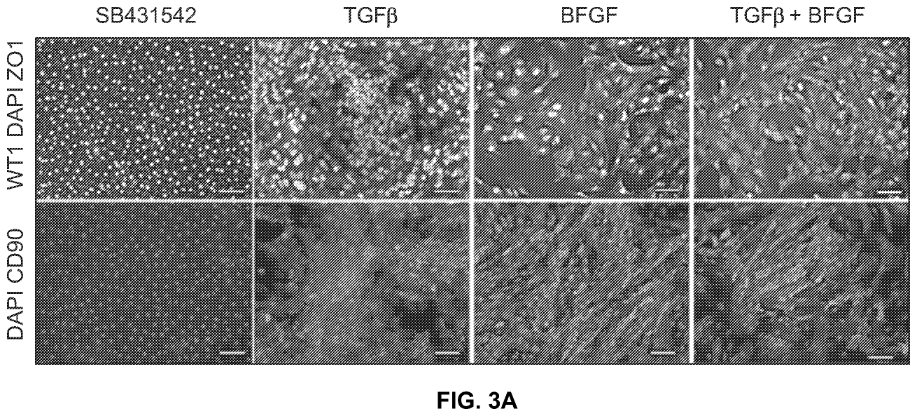

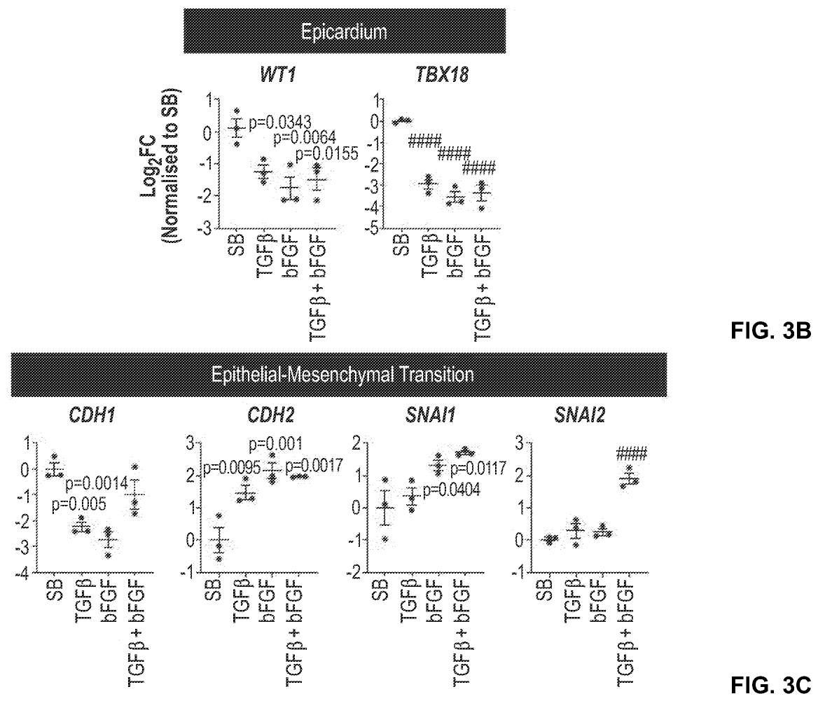

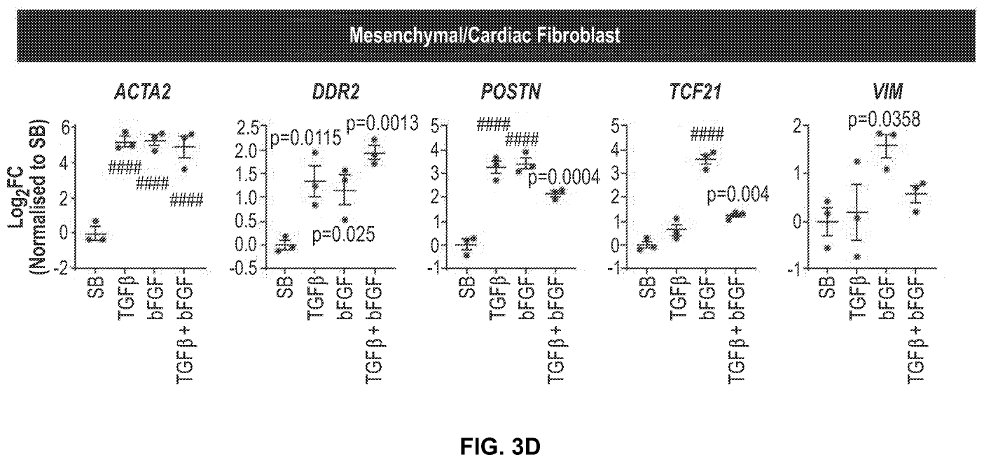

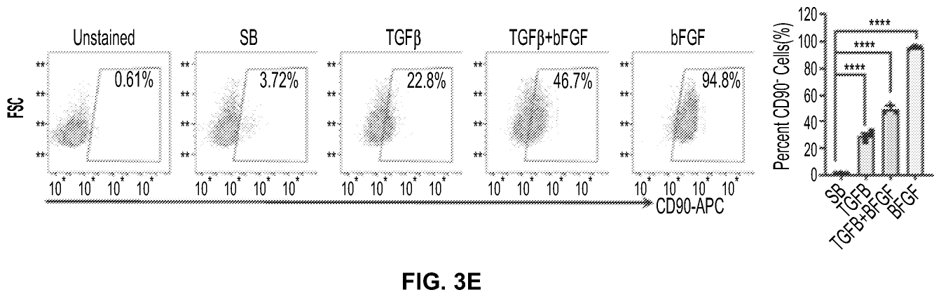

PECs were treated with SB431542 (SB) for TBFβ inhibition, to maintain the cuboidal epithelial phenotype with defined ZO1 expression (FIG. 3A) at each cell border in the extended culture. Without TGFβ inhibition, PECs are prone to spontaneous differentiation into SMA⁺ cells (FIG. 8H), a characteristic which has been described in human fetal epicardial cells (45). Treatment with TGFβ, bFGF, or TGFβ/bFGF for 6 days caused ZO1 disarrangement, cell and nucleus enlargement, and WT1-expression loss in PECs (FIG. 3A). In addition, all three treatments induced new phenotypic expression of mesenchymal marker CD90 (FIG. 3A). The three treatments also significantly downregulated epicardial genes (WT1 and TBX18), E-cadherin gene CDH1 (except TGFβ/bFGF), and conversely upregulated the expression of N-cadherin gene CDH2 (FIGS. 3B-3C), suggesting the loss of epithelial identity. Rapid epithelial-mesenchymal transition (EMT) with upregulation SNAI1 was evident in both bFGF (p=0.0404) and TGFβ/bFGF (p=0.0117) treatments, while SNAI2 was only increased following TGFβ/bFGF treatment. Activation of bFGF, TGFβ, or both pathways demonstrated the potential of PEC transition towards a mesenchymal lineage, with significant increases in ACTA2, DDR2, and POSTN expression (FIG. 3D). bFGF appeared to direct PECs toward a fibroblast fate, with significant upregulation of fibroblast markers TCF21 (p<0.0001) and VIM (p=0.0358). Flow cytometric analysis affirmed mesenchymal commitment as bFGF induced 95.3±0.7% of PECs to be CD90*, compared to SB (1.1±0.1%), TGFβ (28.7±3.5%), or TGFβ+bFGF (49.6±2.2%)(p<0.0001) (FIG. 3E). These collective characteristics are akin to epicardial cells (17, 20).

### Example 4: PECs are Migratory in the Presence of CM, and Able to Undergo EMT in CM Co-Culture

A “complete PEC medium” was formulated containing VEGF, RA, and retinol (abbreviated herein as PECM) for co-culturing PECs and CMs without interfering with TGFβ signaling in the system, while retained WT1⁺ expression, maintained PEC phenotype, and minimized spontaneous EMT events (FIGS. 8G-8H). Culturing PECs in PECM alone showed increased TCF21+ and SMA⁺ cells, along with upregulated TBX18 (p=0.0041), ITGA4 (p<0.0001), and RALDH2 (p=0.0004) in PECs at day 11 (FIG. 8I). Compared with stimulation-suppressed PECs by SB, further upregulation in the epicardial-specific marker uroplakin-1B (UPK1B, p<0.0001), as well as epicardial-related genes TBX18 (p<0.0001), connexin-43 (GJA1, p<0.0001), and a4 integrin (ITGA4, p=0.0002) were observed at days 18 (FIG. 8I). Nonetheless, an upregulation in RALDH2 expression by day 18, the key epicardial maturity marker indicating RA-production capability, was not observed.

For initial characterization of PEC-CM interactions in vitro, the cell types were co-cultured in separate compartments of migration inserts and compared against PEC-only seeded inserts. PEC/CM proliferation was inhibited with mitomycin C (MitoC) to negate confounding effects due to cell proliferation. PECs in co-culture became more migratory by 21 h and recolonized a total area of 119.3±21 μm², while PECs-alone only recolonized a total area of 52.3±12.9 μm² (p=0.0263; FIGS. 10A-10B). Time-lapse monitoring over the course of 10 days showed that mCherry-tagged PECs appeared to invade the Venus-tagged CM layer, causing a change in configuration at the PEC-CM border (FIG. 10Ci, inset, and FIG. 10D). A layer of mCherry⁺, SMA⁺ cells was observed to be residing in between PECs and sarcomeric α-actinin⁺ CMs, while those PECs residing in the remote region remained undifferentiated, with a WT1⁺ SMA− phenotype (FIG. 10Cvi and FIG. 10D). A majority of the re-isolated PECs from CM coculture were found to retain WT1⁺ and TBX18⁺ expression (FIG. 10E).

### Example 5: Direct PEC-CM Co-Culture Enables the Formation of an Integrated Network of Large CM Aggregates

To further understand the effects of cell-cell interactions during PEC-CM co-culture in vitro, we seeded PECs and CMs together without separation in standard, two-dimensional cell culture. Effects were evaluated against comparators of CMs-alone. We assessed co-cultured PEC fate after 6 days by examining PEC gene expression on FACS-selected mCherry⁺ PECs from the mixed population. As compared to PEC only culture, RT-PCR results showed significant upregulation of epicardial markers UPK1B (p=0.0053), ITGA4 (p=0.0417) and ALDH1A2 (p=0.0003), as well as retained CDH1 expression (p=0.7748), confirming the transition of PECs to epicardial cells (FIG. 4A); albeit, without changes in GJA (p=0.0633). Consistently, ECAD⁺, WT1⁺ and CN⁺ cells were also found to present in PEC-CM coculture, by immunostaining (FIG. 4B).

CMs in co-culture formed dense 3D aggregates by day 8, without discernable cell sloughing or death observed during media changes. The aggregates then formed a connected network by day 16, which became larger and more defined by day 21 (FIG. 4C). Seeded with the same number of CMs, CM-only controls appeared to have broader and more even coverage across wells, forming fewer and less-pronounced aggregates (FIG. 4D). Supporting our observations, PEC-CM co-culture significantly condensed CM coverage to 11.79±2.1% of the surface area by day 8, versus 82.48±2.9% coverage in CM-only controls (p<0.0001, FIG. 4E), again without a discernable difference in cell death. Networked CM-aggregates in PEC-CM co-culture showed 18.84±2.7% coverage at day 16, which remained significantly less than 92.65±1.6% coverage in CM-only controls (p<0.0001, FIG. 4E). CM aggregates in PEC-CM co-culture formed larger networks by day 21 with 24.07±2.2% coverage but was not statistically different compared to day 16.

In further characterization of aggregate dimensions, measurements taken by z-plane analysis demonstrated that PEC-CM co-culture aggregates significantly increased in three-dimensional height (106.1±4.8 μm) compared to sparse CM-only aggregates (79.8±3.3 μm, p=0.0001; FIG. 4E). Neighboring 3D aggregates in PEC-CM co-culture appeared to form a connected network, capable of synchronized excitation-contraction coupling under both unstimulated and distant electrical point-stimulation conditions (20 V, 0.6 ms, 0.7 Hz; FIG. 4F).

To test if the process could be reproduced when both cell types developed simultaneously, BVR cocktails that drove PEC formation was integrated into CM differentiation protocol to simulate PEC/CM co-differentiation (FIG. 4G). Striking spontaneous PEC/CM spatial organization was observed at day 9 derived from 50.9% of cTnT+ cells and 39.3% of WT1+ cells (n=1). Layers of WT1+ cells were found surrounding stretches of cTnT+ CMs in tube-like structures in the co-differentiation culture (FIG. 4H). Representative video showed the contracting vCM after 10 days of co-differentiation) and stitched images of a lower magnification showed the overall distribution of cTnT+CM and WT1+ cells in the co-differentiation culture (FIGS. 10F-10G).

To examine if the observed CM aggregation effects were due to space limitation, PEC-CM coculture were evaluated against comparators of CMs co-cultured with human umbilical vein endothelial cells (HUVECs) and human cardiac fibroblasts (HCFs). Within 8 days, PEC-CM co-culture appeared to demonstrate an enhanced degree of spatial CM clustering compared to other culture groups, shown by GFP-selected binary image analysis (FIG. 5A). Quantitative assessment at day 8 found that PEC-CM co-culture significantly reduced CM coverage area (30.4±1.1%, p<0.0001 vs. all groups) compared to CM-alone (78.3±2.8%), HUVEC-CMs (69.0±2.9%), and HCF-CMs (57.6±1.8%) indicating PECs-induced CM compaction (FIG. 5A). Heights of three-dimensional aggregates were also measured by z-plane analysis, in which HUVEC-CMs (34.6±1.3 μm) and HCF-CMs (19.4±0.98 μm) were also significantly less than PEC-CMs (p<0.0001) (FIG. 5B).

### Example 6: PEC-CM Co-Culture Induces Changes in CM Electromechanical Function

In addition to morphometric changes, we observed qualitative changes in contractile function under PEC-CM co-culture conditions. In quantifying these observations, we again compared PEC-CM co-culture to CMs-alone, HUVEC-CM co-culture, and HCF-CM co-culture. Experimental groups were seeded on 1% collagen gels, and CM contractile strain was analyzed using a high-speed imaging algorithm at 14 days under pacing conditions (20 V, 0.6 ms, 0.7 Hz), a technique we have previously used in several in vitro and in vivo cardiac applications (7, 37, 46-49). CMs in PEC-CM co-culture demonstrated significantly more contractile strain (3.89±0.3%, p<0.0001 vs. all groups) compared to CMs-alone (1.74±0.33%), HUVEC-CM co-culture (1.08±0.42%), and HCF-CM co-culture (1.12±0.56%; FIG. 5C). Post-culture collagen gels were analyzed by atomic force microscopy (ΔFM) to determine their stiffness. HUVEC-CM seeded gels demonstrated a higher Young's modulus (1147±86.3 Pa) compared to CMs-alone (381.1±32.1 Pa, p<0.0001), PEC-CMs (660.6±27.7 Pa, p=0.0162), and HCF-CMs (650.4±50.5 Pa, p=0.0001; FIG. 5D). PEC-CM seeded gels had a significantly higher modulus vs. CMs-alone (p=0.0003), but a similar modulus to HCF-CM seeded gels.

Contractility (force/area) was determined to be significantly enhanced for CMs in PEC-CM co-culture (0.029±0.002 mN/mm²) compared to CMs-alone (0.013±0.001 mN/mm², p=0.0354), HUVEC-CM co-culture (0.012±0.002 mN/mm², p=0.0123), and HCF-CM co-culture (0.008±0.001 mN/mm², p<0.0001; FIG. 5E). We hypothesized that differences in contractile mechanics may indicate changes in excitation-contraction coupling. To characterize differences in calcium handling, we incubated all experimental groups with Fluo-3 AM dye to record [Ca²⁺]i transients at day 14, under electrical field stimulation (20 V, 0.6 ms, 0.7 Hz). [Ca²⁺]i transients were determined within each measured region using spatial averaging of fluo-3AM florescence over time, and then analyzed for amplitude, as well as velocity of calcium transient for both upstroke and decay (FIGS. 5F-5I). At day 14, CMs in PEC-CM co-culture demonstrated a directionally higher calcium transient amplitude (ΔF/F0: 0.110±0.007) compared to CMs-alone (ΔF/F0: 0.075±0.009), but the increase did not meet the threshold for statistical significance (p=0.0676). However, the increased PEC-CM calcium transient amplitude was found to be significantly different than both CM-HUVEC (ΔF/F0: 0.043±0.003, p<0.0001) and CM-HCF (ΔF/F0: 0.037±0.002, p<0.0001; FIG. 5F) co-culture groups. Notably, CMs in HUVEC-CM and HCF-CM co-culture demonstrated significantly lower amplitude compared to CMs-alone (p<0.0001). Analyzing calcium transient kinetics, CMs in PEC-CM co-culture demonstrated a significantly increased maximal upstroke velocity (F/F0/sec: 0.0042±0.0003,) compared to CMs-alone (F/F0/sec: 0.0019±0.0003, p=0.0001), HUVEC-CM (F/F0/sec: 0.0018±0.0002, p<0.0001), and HCF-CM (F/F0/sec: 0.0019±0.0002, p=0.0004; FIG. 5G).

CMs in PEC-CM co-culture also demonstrated a directionally higher but not significantly increased maximal decay velocity (F/F0/sec: 0.00037±0.00004) compared to CMs-alone (F/F0/sec: 0.00026±0.00003, p=0.2421). However, the increased PEC-CM maximal decay velocity was found to be significantly higher than both HUVEC-CM (F/F0/sec: 0.00022±0.00002, p=0.0188) and HCF-CM (F/F0/sec: 0.00014±0.00002, p<0.0001; FIG. 5H).

In support of the observed PEC-induced improvement in CM contractility and calcium handling, we further assessed the sarcomere length, cell size and mitochondria content of re-isolated CMs (FIGS. 5J-5L). The average CM sarcomere length after 6 days of PEC co-culture (1.82±0.02 μm; 373 sarcomeres from 16 CMs, from three independent replicates) was found to be significantly longer compared to CMs-alone (1.72±0.03 m, p=0.00264; 230 sarcomeres from 30 CMs, from three independent replicates; FIG. 5J). The percentage of CM with high mitochondria density was also higher after 6 days of PEC co-culture (79.4±3%) vs. CMs-alone (54.9±6%, p=0.0029; FIG. 5K). Nonetheless, there was no significant change in CM cell size after PEC co-culture vs. CM monoculture (FIG. 5I).

### Example 7: PECs Induce CM Proliferation Partly Via the RA-IGF Signaling Axis

PECs are involved in stimulating CM proliferation during heart development, with a more prominent effect in the ventricles (12). To examine this interaction, we first modified our CM differentiation protocol to produce a ventricular myocyte population by inhibiting RA signaling (33, 34) using a potent RXR-α antagonist, BMS189453 (BMS), for 96 h starting at day 3 (FIG. 6A). At day 30, CMs in the BMS-treated group exhibited elongated structure and expressed both MLC2A and MLC2V proteins (FIG. 6B), with significantly upregulated ventricular genes MYH7 (p=0.0089) and MYL2 (p=0.005) as compared to control (FIG. 6A, exhibiting a more ventricular-like identity (34). Consistent with our earlier finding, PEC co-culture caused network formation of CMs after 6 days, and cTnT⁺ EdU⁺ cells were significantly increased (37.2±2.1%) vs. control CMs without PEC co-culture (28.1±1%, p=0.016; FIGS. 6C-6D).

IGF signaling plays a key role in PEC-induced ventricular-CM proliferation during early heart development 4. To test this phenomenon in vitro, ventricular-CMs were treated with Linsitinib (IGF1R inhibitor) for 3 days. Increasing concentrations of Linsitinib gradually and significantly decreased the population of cTnT⁺ EdU⁺ CMs in PEC coculture (21.5±0.8% untreated with 0 nM, to 7.0±0.4% treated with 1 μM, p<0.0001; FIG. 6E), suggesting a significant role of IGF signaling in this process. Furthermore, addition of IGF2 also significantly increased cTnT⁺ EdU⁺ CMs in a dose-dependent order (FIG. 6F). Higher IGF2 protein concentrations were detected in PEC-CM co-culture vs. CM-alone after 2 days (983.2±63 μg/ml vs. 325.7±70 μg/ml, p=0.0011) and after 6 days in culture (1979.7±55.1 μg/ml vs. 242.5 μg/ml, p<0.0001; FIG. 6G) (16). IGF2 level in PECs only culture was similar to CM culture after 2 days (477.9±68.9 μg/ml vs 325.7±69.9 μg/ml) but the level increased to 911.2±16.4 μg/ml/day after 6 days in culture, and the level was significant lower to that of PEC-CM group (p<0.0001).

To study if IGF2 is expressed in PECs and is stimulated by ventricular-CMs, PECs (250,000 cells/well) were co-cultured with increasing numbers of CMs (0 to 500,000 cells/well) in direct co-culture. IGF2 expression increased significantly in FACS re-isolated mCherry-PECs in response to increasing CM number, in which peak IGF2 expression occurred during 1:1 cell-type ratio (250 k PECs: 250 k CMs), eliciting a 2.3-fold increase vs. PEC-alone control (p<0.0001; FIG. 6H). To investigate if IGF2 expression in PECs is RA-signaling dependent, PEC mRNA was analyzed across 3 groups in transwell format: 1) PECs-alone, 2) PEC-CM co-culture, and 3) PEC+BMS in CM co-culture. Normalized to PEC-alone culture, the expression of IGF2 mRNA in co-cultured PECs was upregulated by 107.9-fold. BMS (5 μM) treatment significantly reduced IGF2 expression in co-cultured PECs vs. control without BMS (p=0.0407; FIG. 6I).

### Example 8: Co-Culture of PEC and CM Spheroids Enhances the Morphogenic Complexity within 3D Cardiac Aggregates

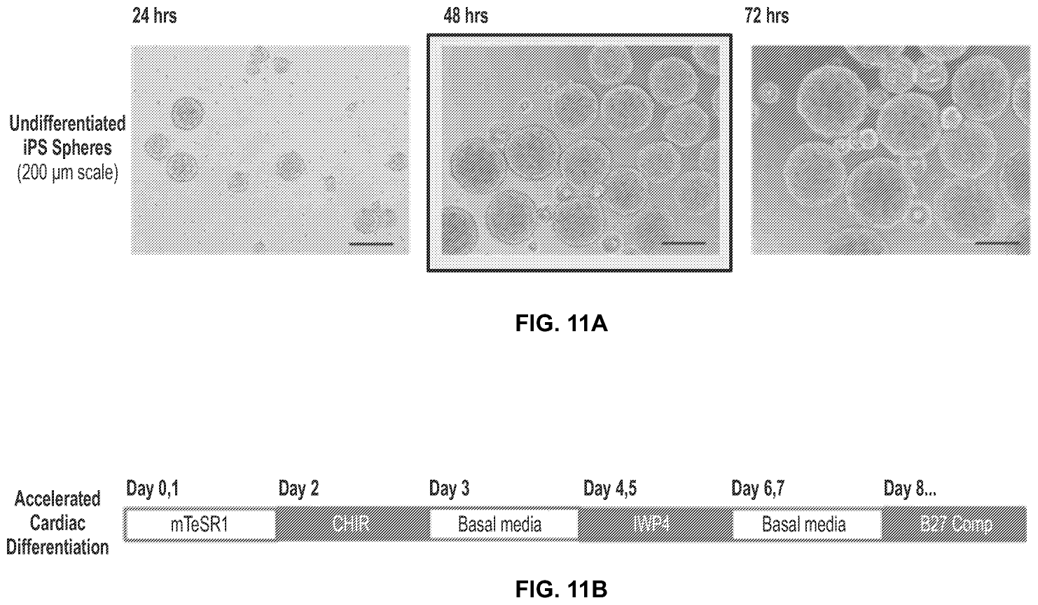

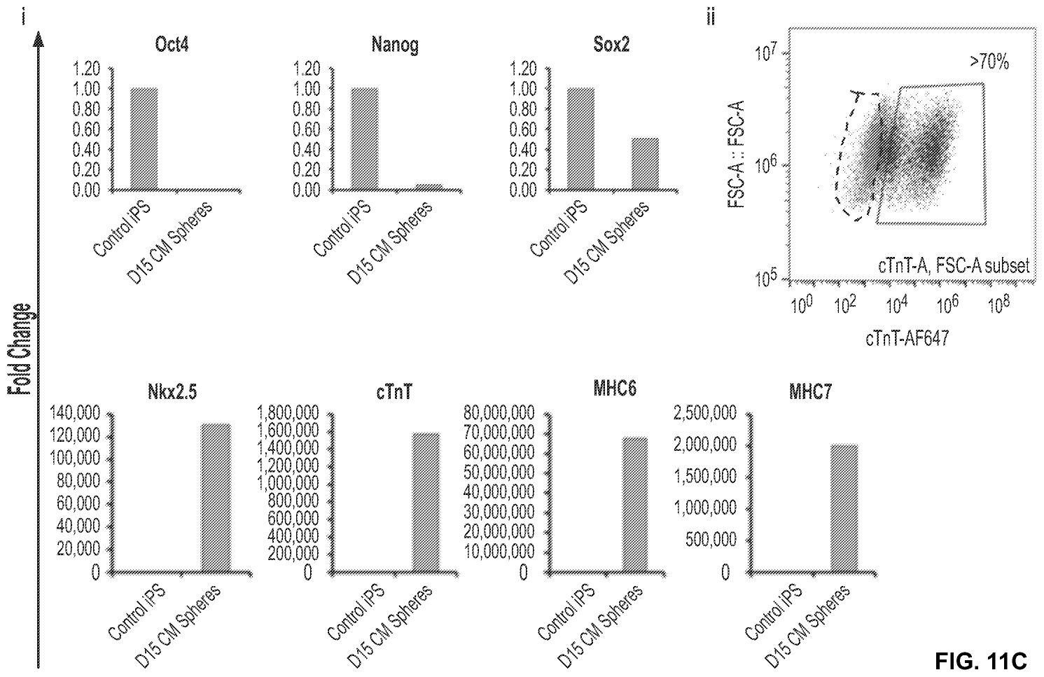

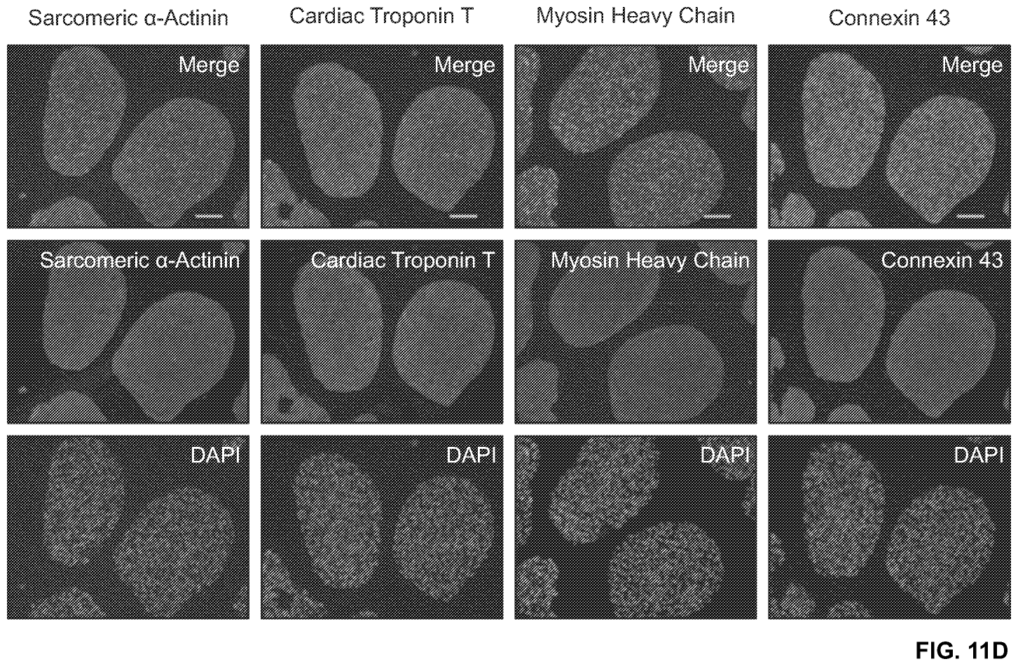

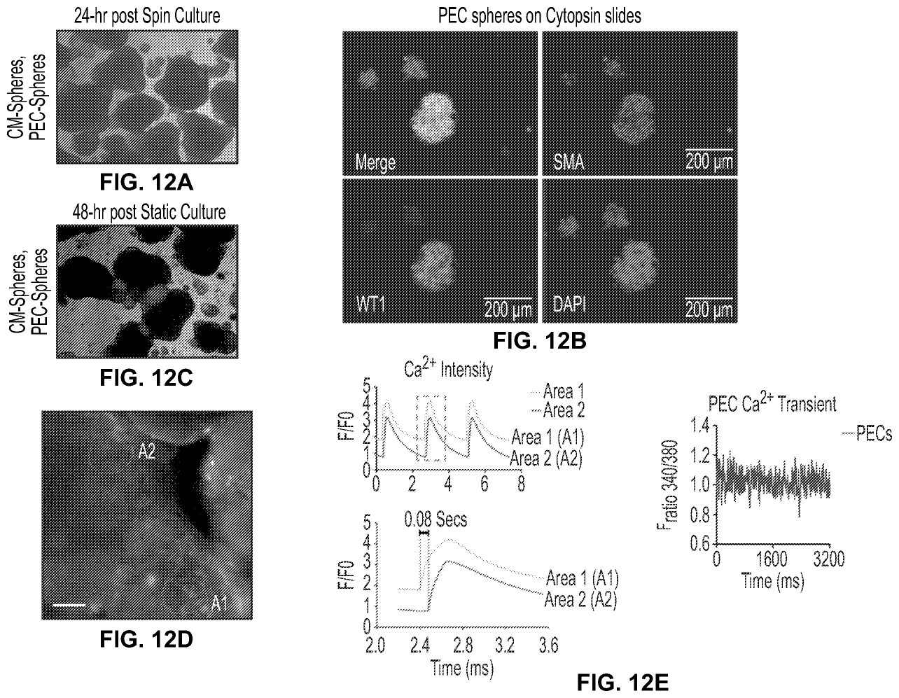

To investigate if the PEC effects on CM functions observed earlier could be reproduced in three-dimensional (3D) culture and recapitulate PEO-to-heart events during development, we sought to incorporate PECs into differentiated CM-spheres. We first employed a facile technique to generate spontaneously beating CM spheroids from hiPSCs in suspension culture (FIGS. 11A-11D), based on previously described techniques. We “inoculated” day 15 CM-sphere spinner flask cultures with day 7 mCherry-PECs (single-cell suspension). Instead of enveloping or integrating with CM-spheres as hypothesized, PECs generated independent spheres after 24 h in spinner flask culture (FIG. 12A). Immunostaining 24-hr PEC-spheres on cytospin slides showed positivity for WT1 and SMA (FIG. 12B). Culturing a sample from the CM/PEC co-sphere suspension in a non-adherent plate under in static conditions, adjacent PEC-spheres and CM-spheres began to connect and integrate within 24 h (FIG. 12C), forming spontaneously beating PEC-CM aggregates and demonstrated excitation-contraction coupling with coupled [Ca2+]i transients and contractile strain (FIG. 12D). Notably, PECs did not demonstrate a calcium transient flux (FIG. 12E).

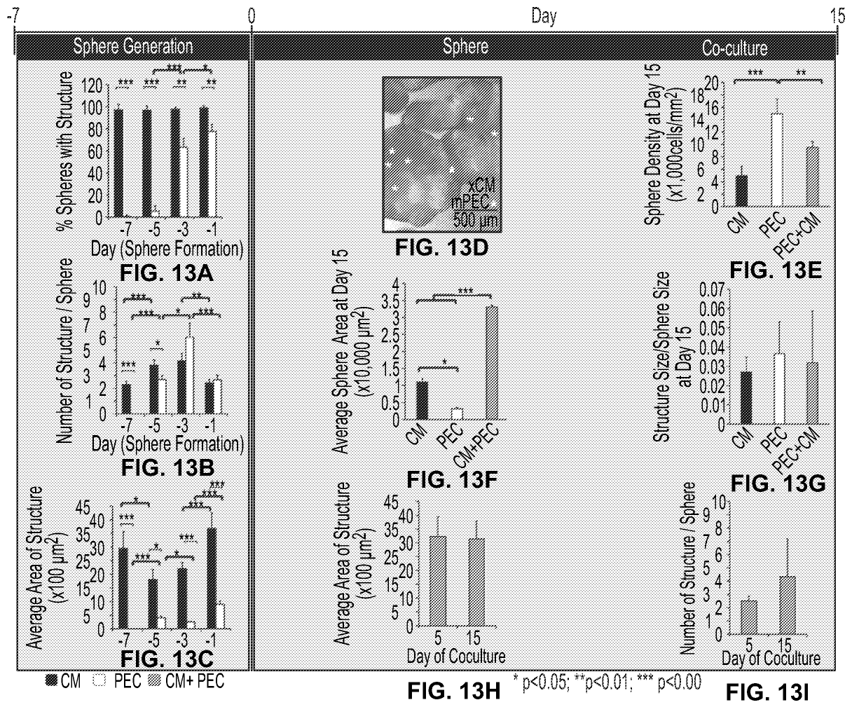

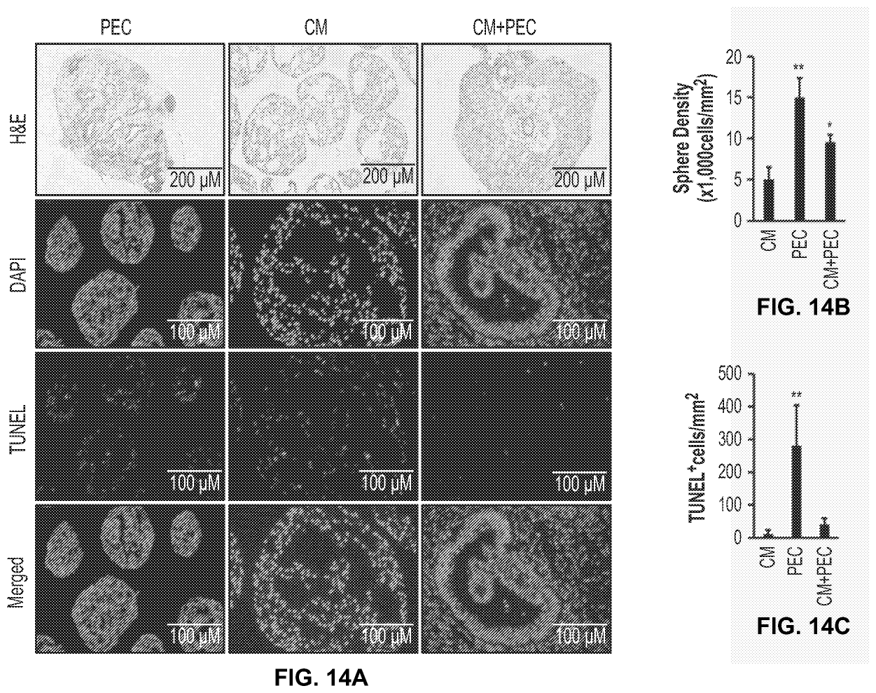

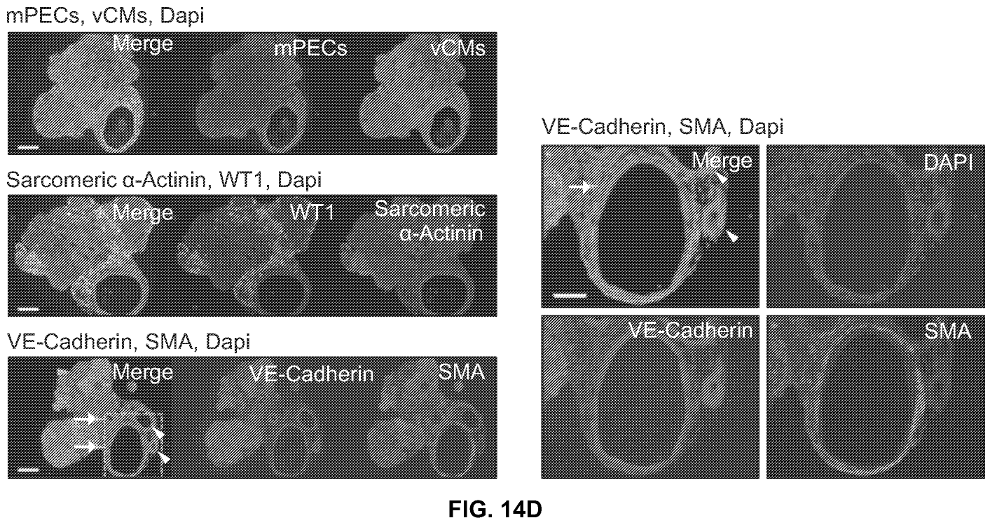

After initial static culture, PEC-CM aggregates could be kept viable under nutation culture with continued beating. The combination of PEC-spheres and CM-spheres into PEC-CM aggregates also appeared to decrease the percentage of TUNEL+ cells (FIGS. 14A and 14C). Under continued nutation culture for 15 days, initial histological analysis of the PEC-CM aggregates by H&E showed the formation of organized luminal structures (FIG. 14A). The luminal structures in PEC-CM aggregates appeared more organized with dense inner-cell layers than “cysts” that have been previously described in culture and CM differentiation of embryoid-bodies (50-52); H&E staining of our CM-spheres also demonstrated the loosely unorganized cystic structures previously described. In-depth characterization of individual PEC- and CM-spheres during its formation, differentiation, and coculture are described in FIGS. 13A-13H.

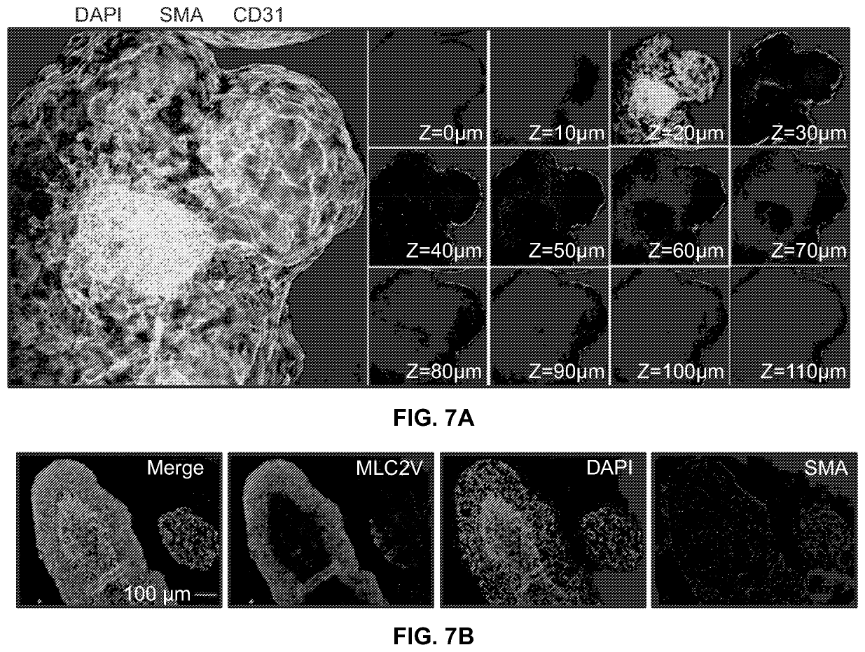

Morphologic analysis of day 15 PEC-CM aggregates (formed with mCherry-PEC-spheres and Venus-CM-spheres) allowed for localization of key features within the morphogenic structures. Based on the distribution of mCherry and Venus-GFP signal, PECs appeared to be integrated with CMs (FIG. 14D). GFP⁺ signal of CMs coincided with a positive sarcomeric α-actinin signal. Undifferentiated PECs demonstrated WT1⁺ expression. Some differentiated PECs appeared to lose WT1 expression but maintained an mCherry⁺ signal, which also co-localized with VE-cadherin⁺ (endothelial) and SMA⁺ (smooth muscle) expression (FIG. 14D). Luminal structures appeared to be highly-organized with densely-ordered cells lining the lumen regardless of size (FIG. 14D inset) vs. cyst-like structures previously described in embryoid body (EB) studies (50-52). Histological analysis revealed there was variation in structure makeup. Some lumens were lined with VE-cadherin⁺ cells (FIG. 14D, triangular arrowheads), as previously seen when EBs are preconditioned with shear stress (53) Yet, other luminal structures were lined with VE-cadherin⁺ cells surrounded by an SMA⁺ cell layer (FIG. 14D, arrows). In a whole-mount staining of PEC-CM aggregates after 15 days in coculture, z-stack analysis confirmed the formation of SMA⁺ cell layer to the adjacent cell aggregates (FIG. 7A). This outer SMA⁺ cell layer was not observed in CM only aggregates which were positive for MLC2V (FIG. 7B).

The proepicardial organ (PEO) is a transient embryonic organ that envelopes the developing heart and eventually forms the epicardium. It drives several developmental events that contribute to cardiac architecture and function including cardiomyocyte proliferation, coronary vessel extension, and myocardial compaction. These capabilities suggest that PEO-derived cells, or the transitioning, intermediate cells prior to forming the epicardial cells (the pre-epicardial cells), play key roles in myocardial regeneration, which are likely critical factors that would facilitate the creation of thick myocardial tissues in vitro.

Studies have shown that BMP4, RA, and Wnt signaling were key mechanisms driving PDGRFA+ and KDR+LPM cells or cardiac progenitors, to a epicardial fate (17, 20), some of which observed better efficiency with simultaneous activation of the signaling (18, 41, 54). The findings here demonstrate the signaling model to develop an efficient BMP4/VEGF/RA based protocol, which generated >86% WT1+ cells in 7 days from monolayer hiPSC culture without the need of embryoid body generation, or fluorescence sorting. Differentiated PECs demonstrated ZO1, TBX18 gene expression, as well as significant upregulation of the transcription factor TBX5 that is critical in proepicardium development and specification (45, 46). PECs also shared similar morphological descriptions and functional characteristics as previously reported, including a cuboidal phenotype after in vitro passaging and capability to undergo EMT (17).

Collectively, the Examples described herein confirm the identity of PECs and their potential to become more mature epicardial cells.

To better the describe the stage of development of the PEC population used in this study, we referred to the most recent publication which delineate the molecular signatures in proepicardial cells in human fetal hearts, a cell population which often refers to the derivation from proepicardial organ capable of migrating to the heart to form epicardium. Comparing the published gene set from human samples, it was found that HEY1, one of the putative markers of proepicardium was upregulated in PECs as compared to EpiH9; whereas C1S, the complement component predominantly expressed in mature epicardial cells, was downregulated in PECs (FIG. 9C). Furthermore, high cTnT− EdU+ cells were observed in CM coculture (FIGS. 6E-6F), which coincides with the GO functional analysis indicating that PECs are more proliferative than the EpiH9 (FIG. 3E). These results also coincide with the observation found by Cui et al. that proepicardial cells are the most actively cycling cells among all non-immune cells in human fetal heart.

The morphologic and functional impact of PECs during development lies in their interactions with the myocardium. Successful application of hiPSC-derived PECs ex vivo or in vivo may require achievement of definitive milestones: formation and maintenance of self-renewing epicardial epithelial layer, derivation of epicardial-derived cells capable of EMT, and activation of biochemical signaling responsible for myocardial developmental features (e.g. proliferation via RA signaling) (55). The Examples described herein demonstrate two of the most fundamental functions that human PECs are capable of, which have not been shown elsewhere using mature human epicardial cells: 1) the ability to form epicardial epithelium, and 2) the ability to express and secrete IGF2 in PEC/CM co-culture, recapitulating aspects of in vivo development. The expression of IGF2 in PEC-derived cells had also increased the number cycling CMs in PEC/CM co-culture.

In well-insert experiments, PECs migrated towards CMs to close the gap within 12-21 h (FIG. 10A), and appeared to form an PEC-layer at 24 h which persisted over 10 days (FIG. 10C). Notably, PECs did not overtake the CM culture, but did appear to consolidate CMs into a more confined area. Direct co-culture of PECs with CMs appeared to consolidate CMs into dense aggregates, significantly reducing CM coverage compared to CMs-alone, HUVEC-CM co-culture, or HCF-CM co-culture within 8 days (FIG. 5A). Previous work in developmental biology has shown that epicardial cells and signaling are necessary for thick ventricular-wall formation through mechanisms of myocardial growth and compaction (11, 12). Without being bound by theory, it is believed that PEC contact or signaling facilitated CM organization into thicker, networked formations (FIG. 4C). Integrating BVR into CM differentiation showed successful derivation of both PEC/CM in single hiPSC culture, and spontaneous PEC/CM spatial organization (FIG. 4E).

Consolidated CM arrangement may also partially account for the observed differences in mechanical function, as densely aggregated CMs provide more contractile units/area working together as a syncytium (FIG. 5A). Under pacing conditions, CMs co-cultured with PECs demonstrated nearly 3× as much contractility (force/area; 0.029±0.002 mN/mm²) than CMs-alone, HUVEC-CM co-culture, or HCF-CM co-culture within 7 days (FIG. 5E). In 2-dimensional culture, the PEC-CM combination generated contractility on the order of engineered myocardial tissue (56). Beyond the suggestion that the mechanical differences are solely due to condensed contractile CMs, it may also be possible that PECs may have induced CM-proliferation to add more contractile units, as evidenced by the results in FIG. 6C-6D. As CMs in PEC co-culture demonstrated longer, more mature sarcomeres and increase mitochondria content, it is also possible that PEC-CM co-culture could have facilitated increased contractility through either improved length-dependent CM activation or enhanced CM maturity (FIG. 5J-5K). Nonetheless, the CM size did not significantly differ in the presence of PECs. This observation could be a result of increased small CM population due to proliferation or the short coculture time period (6 days).

In evaluation of calcium handling, CMs in PEC-CM co-culture demonstrated significantly increased [Ca2+]i transient amplitudes compared to other co-culture groups, as well as directional improvement vs. CMs-alone (FIG. 5F). In analyzing [Ca2+]i transient kinetics, PEC-cocultured CMs also showed significantly increased maximal upstroke vs. all other groups (FIG. 5G); while decay velocity was significantly increased compared to HUVEC-CMs and HCF-CMs, and directionally improved vs. CMs-alone (FIG. 5H). Taken together, these results suggest that CMs co-cultured with PECs may have more efficient calcium handling, especially in consideration of the enhanced mechanical contractility. Increased calcium transient amplitudes observed in PEC-CM co-culture could suggest that PEC-induced CMs have increased Ca2+ release by sarcoplasmic reticulum—a mechanism that could support enhanced inotropy.

In a simplified in vitro model of three-dimensional tissue, we generated CM-spheres and PEC-spheres, and then combined them to for PEC-CM aggregates. After 15 days of sphere co-culture, H&E staining of the PEC-CM aggregates showed well-organized phenotypically-complex luminal structures (FIG. 14) that appeared to be distinct from cystic structures previously described in embryoid-body studies (50-52) and more indicative of evidence of high-order architecture and cellularity (53, 57, 58).

In day 15 PEC-sphere and CM-sphere controls, PEC-spheres also contained several well-organized luminal structures, while CM-spheres generated typical cystic structures. Analyzing the development of these structures days −7 to −1 prior to sphere co-culture (FIG. 13), the immediate presentation and persistence of CM-sphere cysts would suggest these could result from culture conditions (e.g. spin-culture speed, diffusion/necrosis) (53, 59). However, the gradual development of more complex PEC-sphere structures would suggest more sophisticated mechanical (e.g. spin-culture) and biochemical (e.g. EMT) interactions. Through serial-section immunostaining of PEC-CM aggregates, PEC-derived cells were observed that had undergone EMT to generate SMA+ or VE-cadherin+ cells in the luminal structures (FIG. 14D).

These results coincide with additional evidence that PECs can undergo EMT to demonstrate SMA, vimentin, periostin and CD90 markers when co-cultured with CMs in 2D culture (FIGS. 2B-2D). The observation that VE-cadherin+ endothelial-like cells and E-cadherin+ epithelial-like cells can integrate within luminal structures could indicate that cell-cell communication plays a key role in formation, order, or purpose of the structures (FIG. 14D). Alternatively, it also is possible that functions of the luminal structures could influence surrounding cell phenotypes.

Taken together, these findings suggests that hiPSC-derived PECs are functional and able to interact with CMs to enhance the function and structural organization in 3-dimensional PEC/CM microtissues, and the strategic combination of early-stage cardiac cell types can enable the creation of more sophisticated and mature cardiac grafts.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Montero P. et al., Cells, Materials, and Fabrication Processes for Cardiac Tissue Engineering Front. Bioeng. Biotechnol., 11 Aug. 2020
2. Vunjak-Novakovic G, et al. Challenges in cardiac tissue engineering. Tissue Eng Part B Rev. 2010; 16(2):169-187
3. Katz T C, et al. Distinct compartments of the proepicardial organ give rise to coronary vascular endothelial cells. Dev Cell 22, 639-650 (2012).
4. Laflamme M A, et al. Cardiomyocytes derived from human embryonic stem cells in pro-survival factors enhance function of infarcted rat hearts. Nat Biotechnol 25, 1015-1024 (2007).
5. Lian X, et al. Robust cardiomyocyte differentiation from human pluripotent stem cells via temporal modulation of canonical Wnt signaling. Proc Natl Acad Sci USA 109, E1848-1857 (2012).
6. Burridge P W, Keller G, Gold J D, Wu J C. Production of de novo cardiomyocytes: human pluripotent stem cell differentiation and direct reprogramming. Cell Stem Cell 10, 16-28 (2012).
7. Guyette J P, et al. Bioengineering Human Myocardium on Native Extracellular Matrix. Circ Res 118, 56-72 (2016).
8. Mummery C L, Zhang J, Ng E S, Elliott D A, Elefanty A G, Kamp T J. Differentiation of human embryonic stem cells and induced pluripotent stem cells to cardiomyocytes: a methods overview. Circ Res 111, 344-358 (2012).
9. Veerman C C, Kosmidis G, Mummery C L, Casini S, Verkerk A O, Bellin M. Immaturity of human stem-cell-derived cardiomyocytes in culture: fatal flaw or soluble problem? Stem Cells Dev 24, 1035-1052 (2015).
10. Yang X, Pabon L, Murry C E. Engineering adolescence: maturation of human pluripotent stem cell-derived cardiomyocytes. Circ Res 114, 511-523 (2014).
11. Weeke-Klimp A, et al. Epicardium-derived cells enhance proliferation, cellular maturation and alignment of cardiomyocytes. J Mol Cell Cardiol 49, 606-616 (2010).
12. Merki E, et al. Epicardial retinoid X receptor alpha is required for myocardial growth and coronary artery formation. Proc Natl Acad Sci USA 102, 18455-18460 (2005).
13. Martinez-Estrada O M, et al. Wt1 is required for cardiovascular progenitor cell formation through transcriptional control of Snail and E-cadherin. Nat Genet 42, 89-93 (2010).
14. Smith C L, Baek S T, Sung C Y, Tallquist M D. Epicardial-derived cell epithelial-to-mesenchymal transition and fate specification require PDGF receptor signaling. Circ Res 108, e15-26 (2011).
15. Ieda M, et al. Cardiac fibroblasts regulate myocardial proliferation through beta1 integrin signaling. Dev Cell 16, 233-244 (2009).
16. Cao J, Poss K D. The epicardium as a hub for heart regeneration. Nat Rev Cardiol 15, 631-647 (2018).
17. Witty A D, et al. Generation of the epicardial lineage from human pluripotent stem cells. Nat Biotechnol 32, 1026-1035 (2014).
18. Iyer D, et al. Robust derivation of epicardium and its differentiated smooth muscle cell progeny from human pluripotent stem cells. Development 143, 904 (2016).
19. Tan J J, Guyette J P, Xiao L, Milan D J, Ott H C. Abstract 20554: Human iPS Derived Proepicardial Cells Enhance Cardiomyocyte Function and Organoid Structure. Circulation 136, A20554-A20554 (2017).
20. Bao X, et al. Long-term self-renewing human epicardial cells generated from pluripotent stem cells under defined xeno-free conditions. Nat Biomed Eng 1, (2016).
21. Tran F H, Zheng J J. Modulating the wnt signaling pathway with small molecules. Protein Sci. 2017; 26(4):650-661.
22. Zheng S, et al. Small-molecule inhibitors of Wnt signaling pathway: towards novel anticancer therapeutics. Future Med Chem. 2015; 7(18):2485-2505.
23. Freemantle S J, et al. Developmentally-related candidate retinoic acid target genes regulated early during neuronal differentiation of human embryonal carcinoma. Oncogene. 2002 Apr. 25; 21(18):2880-9. doi: 10.1038/sj.onc.1205408.
24. Savory J G A et al et al, Identification of novel retinoic acid target genesDev Biol. 2014 Nov. 15; 395(2):199-208
25. Balmer J E, Blomhoff R. Gene expression regulation by retinoic acid. J Lipid Res. 2002 November; 43(11):1773-808.
26. Edgar R, Domrachev M, Lash A E. Gene Expression Omnibus: NCBI gene expression and hybridization array data repository. Nucleic Acids Res 30, 207-210 (2002).
27. Barrett T, et al. NCBI GEO: archive for functional genomics data sets—update. Nucleic Acids Res 41, D991-995 (2013).
28. Bolger A M, Lohse M, Usadel B. Trimmomatic: a flexible trimmer for Illumina sequence data. Bioinformatics 30, 2114-2120 (2014).
29. Ewels P, Magnusson M, Lundin S, Kaller M. MultiQC: summarize analysis results for multiple tools and samples in a single report. Bioinformatics 32, 3047-3048 (2016).
30. Li H, Durbin R. Fast and accurate long-read alignment with Burrows-Wheeler transform. Bioinformatics 26, 589-595 (2010).
31. Barnett D W, Garrison E K, Quinlan A R, Stromberg M P, Marth G T. BamTools: a C++ API and toolkit for analyzing and managing BAM files. Bioinformatics 27, 1691-1692 (2011).
32. Liao Y, Smyth G K, Shi W. featureCounts: an efficient general purpose program for assigning sequence reads to genomic features. Bioinformatics 30, 923-930 (2014).
33. Ge S X, Son E W, Yao R. iDEP: an integrated web application for differential expression and pathway analysis of RNA-Seq data. BMC Bioinformatics 19, 534 (2018).
34. Robinson M D, McCarthy D J, Smyth G K. edgeR: a Bioconductor package for differential expression analysis of digital gene expression data. Bioinformatics 26, 139-140 (2010).
35. Love M I, Huber W, Anders S. Moderated estimation of fold change and dispersion for RNA-seq data with DESeq2. Genome Biol 15, 550 (2014).
36. Ashburner M, et al. Gene ontology: tool for the unification of biology. The Gene Ontology Consortium. Nat Genet 25, 25-29 (2000).
37. Kelly D J, Azeloglu E U, Kochupura P V, Sharma G S, Gaudette G R. Accuracy and reproducibility of a subpixel extended phase correlation method to determine micron level displacements in the heart. Med Eng Phys 29, 154-162 (2007).
38. Hansen K J, et al. Functional Effects of Delivering Human Mesenchymal Stem Cell-Seeded Biological Sutures to an Infarcted Heart. Biores Open Access 5, 249-260 (2016).
39. Tao Z W, et al. Delivering stem cells to the healthy heart on biological sutures: effects on regional mechanical function. Journal of tissue engineering and regenerative medicine, (2014).
40. Chong J J, et al. Human embryonic-stem-cell-derived cardiomyocytes regenerate non-human primate hearts. Nature 510, 273-277 (2014).
41. Guadix J A, et al. Human Pluripotent Stem Cell Differentiation into Functional Epicardial Progenitor Cells. Stem Cell Reports 9, 1754-1764 (2017).
42. Cui Y, et al. Single-Cell Transcriptome Analysis Maps the Developmental Track of the Human Heart. Cell Reports 26, 1934-1950.e1935 (2019).
43. Zhao M, Kong L, Liu Y, Qu H. dbEMT: an epithelial-mesenchymal transition associated gene resource. Sci Rep 5, 11459 (2015).
44. Zhao M, Liu Y, Zheng C, Qu H. dbEMT 2.0: An updated database for epithelial-mesenchymal transition genes with experimentally verified information and precalculated regulation information for cancer metastasis. Journal of Genetics and Genomics 46, 595-597 (2019).
45. Moerkamp A T, et al. Human fetal and adult epicardial-derived cells: a novel model to study their activation. Stem Cell Res Ther 7, 174 (2016).
46. Gaudette G R, Todaro J, Krukenkamp I B, Chiang F P. Computer aided speckle interferometry: a technique for measuring deformation of the surface of the heart. Ann Biomed Eng 29, 775-780 (2001).
47. Kelly D J, et al. Increased myocyte content and mechanical function within a tissue-engineered myocardial patch following implantation. Tissue engineering Part A 15, 2189-2201 (2009).
48. Tao Z W, et al. Delivering stem cells to the healthy heart on biological sutures: effects on regional mechanical function. J Tissue Eng Regen Med 11, 220-230 (2017).
49. Hansen K J, et al. Functional Effects of Delivering Human Mesenchymal Stem Cell-Seeded Biological Sutures to an Infarcted Heart. Biores Open Access 5, 249-260 (2016).
50. Carpenedo R L, Sargent C Y, McDevitt T C. Rotary suspension culture enhances the efficiency, yield, and homogeneity of embryoid body differentiation. Stem Cells 25, 2224-2234 (2007).
51. Bratt-Leal A M, Carpenedo R L, McDevitt T C. Engineering the embryoid body microenvironment to direct embryonic stem cell differentiation. Biotechnol Prog 25, 43-51 (2009).
52. Kulinski T M, et al. Imprinted expression in cystic embryoid bodies shows an embryonic and not an extra-embryonic pattern. Dev Biol 402, 291-305 (2015).
53. Nsiah B A, Ahsan T, Griffiths S, Cooke M, Nerem R M, McDevitt T C. Fluid shear stress pre-conditioning promotes endothelial morphogenesis of embryonic stem cells within embryoid bodies. Tissue engineering Part A 20, 954-965 (2014).
54. Zhao J, et al. Efficient Differentiation of TBX18(+)/WT1(+) Epicardial-Like Cells from Human Pluripotent Stem Cells Using Small Molecular Compounds. Stem Cells Dev 26, 528-540 (2017).
55. Chen T, et al. Epicardial induction of fetal cardiomyocyte proliferation via a retinoic acid-inducible trophic factor. Dev Biol 250, 198-207 (2002).
56. Ruan J L, et al. Mechanical Stress Conditioning and Electrical Stimulation Promote Contractility and Force Maturation of Induced Pluripotent Stem Cell-Derived Human Cardiac Tissue. Circulation 134, 1557-1567 (2016).
57. Tomov M L, Olmsted Z T, Paluh J L. The Human Embryoid Body Cystic Core Exhibits Architectural Complexity Revealed by use of High Throughput Polymer Microarrays. Macromol Biosci 15, 892-900 (2015).
58. Ditadi A, et al. Human definitive haemogenic endothelium and arterial vascular endothelium represent distinct lineages. Nat Cell Biol 17, 580-591 (2015).
59. Sargent C Y, et al. Hydrodynamic modulation of embryonic stem cell differentiation by rotary orbital suspension culture. Biotechnol Bioeng 105, 611-626 (2010).

1. A method of generating a population of pre-epicardial cells (PECs), the method comprising:
providing a population of induced pluripotent stem cells (iPSCs), preferably human iPSCs;
culturing the population of cells at a density of about 5,000-500,000 cells per mm², preferably about 200,000 cells per mm²;
performing the following steps in order:
(a) treating the cells with a first medium comprising a Wnt signaling activator for about 1-4 days in the absence of insulin, preferably for about 48 hours;
(b) replacing the first medium of step (a) with a second medium comprising insulin for about 24 hours; and
(c) treating the cells with one or more of Bone Morphogenetic Protein 4 (BMP4), Retinoic Acid, and optionally vascular endothelial growth factor (VEGF), for about 3-7 days, preferably for about 4 days.

2. The method of claim 1, wherein the method further comprises the following steps in order between steps (b) and (c):
(i) treating the cells with a Wnt signaling inhibitor for about 1-3 days, preferably about 48 hours in the second medium; and
(ii) replacing the second medium with a third medium comprising insulin for about 1-3 days, preferably for about 48 hours.

3. A method of generating a population of pre-epicardial cells (PECs) and cardiomyocytes (CMs), the method comprising:
providing a population of induced pluripotent stem cells (iPSCs), preferably human iPSCs;
culturing the population of cells at a density of about 5,000-500,000 cells per mm², preferably about 200,000 cells per mm²;
performing the following steps in order:
(a) treating the cells with a first medium comprising a Wnt signaling activator for about 1-4 days in the absence of insulin, preferably for about 48 hours;
(b) replacing the first medium of step (a) with a second medium comprising insulin for about 24 hours;
(c) treating the cells with a Wnt signaling inhibitor for about 1-3 days, preferably about 48 hours in the second medium; and
(d) replacing the second medium with a third medium comprising insulin for about 1-3 days, preferably for about 48 hours; and
(e) treating the cells with one or more signaling activators of Bone Morphogenetic Protein 4 (BMP4), Retinoic Acid, and optionally vascular endothelial growth factor (VEGF), for about 3-7 days, preferably about 4 days.

4. The method of claim 1, wherein the first and/or second medium is a serum-free medium.

5. The method of claim 4, wherein the first and/or second medium is Roswell Park Memorial Institute (RPMI) 1640 medium.

6. The method of claim 4, wherein the first medium RPMI medium with B-27 Supplement Minus Insulin.

7. The method of claim 1, wherein the Wnt signaling activator is provided in a range of about 8 to about 15 μM, preferably about 12 μM.

8. The method of claim 1, wherein the BMP4 is provided in a range of about 25 to about 75 ng/ml, preferably about 50 ng/ml.

9. The method of claim 1, wherein the VEGF is provided in a range of about 2 to about 7 ng/ml, preferably about 5 ng/ml.

10. The method of claim 1, wherein the retinoic acid is provided in a range of about 2 to about 6 μM, preferably about 4 μM.

11. The method of claim 2, wherein the Wnt signaling inhibitor is provided in a range of about 2 to about 7 μM, preferably about 5 μM.

12. The method of claim 1, wherein the Wnt signaling activator is CHIR99021.

13. The method of claim 2, wherein the Wnt signaling inhibitor is IWP-4.

14. The method of claim 1, wherein the PECs express one or more of the markers WT1, TBX18, SEMA3D and SCX within 7 days of generating PECs.

15. The method of claim 2, wherein the PECs express one or more of the markers UPK1B, ITGA4, ALDH1A2 after 7 days of being generated, wherein the PECs are contact with CMs.

16. The method of claim 2, wherein the PECs have one or more of the follow characteristics:
(1) secrete IGF2;
(2) stimulate CM proliferation; and
(3) induce the formation of functional CM aggregates.

17. A population of cells comprising preferably at least 60%, 70%, 80%, or 90% PECs made by the method of claim 1.

18. A population of cells comprising PECs and CMs made by the method of claim 2.

19. A composition comprising the population of cells of claim 17.

20. A method of treating a subject who has or is at risk of developing a cardiovascular disease or has injured myocardial tissue, the method comprising:
obtaining primary somatic cells, preferably from the subject who has or is at risk of developing cardiovascular disease, and generating iPSCs from the primary cells;
generating a population of cells comprising PECs and optionally CMs by the method of claim 1; and
administering the population of cells to the subject.

21. The method of claim 20, wherein the cells are administered by being implanted directly into or near the affected area of the subject's heart.

22. The method of claim 21, wherein the cells are administered directly via injection.

23. The method of claim 20, wherein the cells are placed onto one or more degradable sheets implanted on the subject's heart.

24. The method of claim 20, wherein administration of the cells improves cardiac functionality.
